(19) United States
(12) Patent Application Publication
(10) Pub. No.: US 2024/0317732 A1
(43) Pub. Date: Sep. 26, 2024

(54) A NEW MOLECULAR SCAFFOLD FOR TARGETING HRPN13

(71) Applicant: THE UNITED STATES OF AMERICA AS REPRESENTED BY THE SECRETARY, DEPARTMENT OF HEALTH AND HUMAN SERVICE, Rockville, MD (US)

(72) Inventors: Kylie J. Walters, Frederick, MD (US); Xiuxiu LU, Frederick, MD (US); Nadya I. Tarasova, Frederick, MD (US); Eric Rolf SWENSON, Silver Spring, MD (US); Venkatareddy SABBASANI, Rockville, MD (US); Beverly Ann MOCK, Bethesda, MD (US); Snehal GAIKWAD, Bethesda, MD (US); Deborah CITRIN, Bethesda, MD (US); Monika CHANDRAVANSHI, Frederick, MD (US)

(21) Appl. No.: 18/262,941

(22) PCT Filed: Jan. 28, 2022
(86) PCT No.: PCT/US22/14199

§ 371 Date: Jul. 26, 2023

Related U.S. Application Data
(60) Provisional application No. 63/143,398, filed on Jan. 29, 2021.

(51) Int. Cl. C07D 417/14; C07C 255/44; C07C 311/46; C07D 213/81; C07D 401/14; C07D 403/12; C07D 417/12

(52) U.S. Cl. CPC C07D 417/14; C07C 255/44; C07C 311/46; C07D 213/81; C07D 401/14; C07D 403/12; C07D 417/12

(57) ABSTRACT

In accordance with the purpose(s) of the present disclosure, as embodied and broadly described herein, the disclosure, in one aspect, relates to scaffold molecules having anti-hRPN13 activity, proteolysis targeting chimeras (PROTACs) incorporating the same, methods of making same, pharmaceutical compositions comprising same, and methods of treating cancers involving aberrant hRpn13 activity and/or the presence of hRpn13-Pru/hRpn13 or variants thereof using the same.

### CROSS REFERENCE TO RELATED APPLICATIONS

This application claims the benefit of U.S. Provisional Application No. 63/143,398 filed on Jan. 29, 2021, which is incorporated herein by reference in its entirety.

### BACKGROUND

The 26S proteasome is formed by a regulatory particle (RP) that binds and processes ubiquitinated substrates and a core particle (CP) that hydrolyzes proteins into peptides. CP inhibitors are used to treat hematological cancers but emerged cases of resistance by mutations in the targeted subunit motivate new strategies for proteasome inhibition. Proteasome substrates are marked by covalently attached ubiquitin chains and the therapeutic potential of the ubiquitin-proteasome pathway in cancer treatment has exploded with new possibilities by invoking Proteolysis Targeting Chimeras (PROTACs), which link molecular targets to ubiquitination machinery.

Rpn1, Rpn10, and Rpn13 in the RP bind ubiquitin or a shuttle factor carrying ubiquitinated substrates as well as ubiquitin-processing enzymes; namely, deubiquitinating enzymes UCHL5/Uch37 and Usp14/Ubp6 for hRpn13 and hRpn1 respectively and E3 ligase E6AP/UBE3A for hRpn10. In addition to UCHL5 and Usp14, the proteasome RP has an essential deubiquitinating enzyme, Rpn11. Positioned near the substrate entrance, Rpn11 couples the removal of ubiquitin chains with substrate translocation through the center of the proteasome ATPase ring by direct interaction with substrate-conjugated ubiquitin chains. Rpn11 interaction with ubiquitin chains at the proteasome does not require substrate; thus, it likely plays an active role in positioning ubiquitinated substrates proximal to the nearby ATPase ring. Multiple inhibitors have been developed against Rpn11 that block cancer cell proliferation, induce the unfolded protein response, and/or trigger apoptosis.

CRISPR-based gene editing indicated hRpn13-binding compounds (RA190 and RA183) to induce apoptosis in an hRpn13-dependent manner, albeit knockdown experiments suggest little dependency, including for an hRpn13-binding peptoid. The C-terminal end of proteasome subunit hRpn2 extends across the hRpn13 N-terminal Pru (Pleckstrin-like receptor for ubiquitin) domain which also binds ubiquitin dynamically, maintaining it in an extended conformation, with interactions at the ubiquitin linker region that cause preference for chains linked by K48. RA190 and RA183 react with hRpn13 C88 at the periphery of the hRpn2-binding region, but are generally reactive with exposed cysteines, impairing specificity.

Aberrant hRpn13 activity has been implicated in a number of human cancers, including, but not limited to, multiple myeloma, lymphoma, mantle cell lymphoma, acute leukemia, cancers associated with human papillomavirus, colorectal cancer, gastric cancer, ovarian cancer, liver cancer, breast cancer, cervical cancer, prostate cancer, and pancreatic cancer. Although some hRpn13 binding compounds have been developed and have shown some efficacy, these compounds have displayed some degree of cytotoxicity or other off-target effects or have required high dosages that may lead to systemic side effects in clinical applications and/or false positives in diagnostic assays. It would be advantageous to develop new hRpn13-targeting molecules including small molecule scaffolds and PROTACs that have greater specific activity against hRpn13, in order to more effectively treat hRpn13-associated cancers.

Despite advances in research targeting hRpn13 degradation, there is still a scarcity of compounds that are potent, efficacious, and selective inhibitors and/or PROTACs of hRpn13 that are also effective in the treatment of cancers associated with aberrant hRpn13 activity and/or that exhibit the presence of hRpn13 variants, including truncated variants that contain the hRpn13 N-terminal Pleckstrin-like receptor for ubiquitin (Pru) domain and not the C-terminal UCHL5-binding domain (RPN13-Pru). These needs and other needs are satisfied by the present disclosure.

### SUMMARY

In accordance with the purpose(s) of the present disclosure, as embodied and broadly described herein, the disclosure, in one aspect, relates to scaffold molecules having anti-hRPN13 activity, proteolysis targeting chimeras (PROTACs) incorporating the same, methods of making same, pharmaceutical compositions comprising same, and methods of treating cancers involving aberrant hRpn13 activity and/or the presence of hRpn13-Pru/hRpn13^{Pru} or variants thereof using the same.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1D show an in silico screen that identifies an hRpn13-binding compound. FIG. 1A, Emission at 350 nm for 1 μM hRpn13 Pru with addition of XL5 (bottom line) or RA190 (top line). The plots depict mean±SD from three parallel recordings above which chemical structures are included. FIG. 1B, ¹H, ¹⁵N HSQC spectra of 20 μM ¹⁵N-hRpn13 Pru (darker peaks) or 250 μM ¹⁵N-hRpn13 Pru with 2-fold molar excess XL5 (lighter peaks) in NMR buffer at 10° C., with an expansion for clarity. Arrows highlight the shifting of hRpn13 signals from their free state to their XL5-bound state. Residue signals that disappear (italicized) or V38, which appears, following XL5 addition are labeled. FIG. 1C, hRpn13 amino acids significantly affected by XL5 addition in (FIG. 1B) are labeled and shown in ribbon format (α helix and β sheet) on a secondary structure diagram of the hRpn13 Pru (ribbon format):hRpn2 (940-953) (stick format) complex (PDB 6CO4). hRpn13 residues shifted by greater than one standard deviation above average or that appear (V38) or disappear following XL5 addition are labeled. hRpn2 side chain heavy atoms are displayed and key amino acids labeled. FIG. 1D, ITC analysis of hRpn13 binding to XL5. Raw ITC data (top) from titration of 200 μM hRpn13 Pru into 20 μM XL5 with the binding isotherm and fitted thermodynamic values (bottom).

FIGS. 2A-2C show XL5 covalently targets hRpn13 and induces cell death. FIG. 2A, LC-MS analysis of 2 μM purified hRpn13 Pru (MW: 17017.3 g/mol) incubated with 20 μM XL5 for 2 hours at 4° C. The resulting compound adduct and unmodified hRpn13 Pru are labeled along with the detected molecular weight (Da). FIG. 2B, LC-MS analysis of 40 μM XL5 incubated with 2 mM reduced L-glutathione (GSH, MW: 307.3 g/mol) for 2 hours at 4° C. Detected GSH adducts are indicated and a table is included that lists relative abundance. FIG. 2C, HCT116 WT (center line), HCT116 trRpn13 (top line) or RPMI 8226 (bottom line) cells were treated with the indicated concentration of XL5 for 48 hours and cell metabolism measured by an MTT assay (mean±SD). Viability is plotted as (I₅₇₀)ₛₐₘₚₗₑ/(I₅₇₀)_{control}×100(%).

FIGS. 3A-3E show the structure and associated data of XL5-ligated hRpn13 Pru. FIGS. 3A-3B, Chemical structure of XL5 (left panel) ligated to the sulfur atom from hRpn13 C88. Hydrogen atoms are labeled with numbers used in the text and figures. Selected regions from a ¹H, ¹³C half-filtered NOESY (100 ms) experiment (FIG. 3A, right panel and FIG. 3B) acquired on a sample containing 0.25 mM ¹³C-labeled hRpn13 Pru and 2-fold molar excess unlabeled XL5 dissolved in NMR buffer. FIG. 3C, Chemical structure of XL5-¹³C₆-BA (left panel) illustrating ¹³C-labeling. Selected regions from a ¹H, ¹³C half-filtered NOESY (100 ms) experiment (right panel) acquired on a sample with 0.4 mM unlabeled hRpn13 Pru and equimolar of XL5-¹³C₆-BA dissolved in NMR buffer containing 70% 2H₂O. FIG. 3D, Structural ensemble (left panel) or ribbon diagram (right panel) of hRpn13 (ribbon format) ligated to XL5 (stick format) with C15 and C16 in the SS stereoconfiguration. hRpn13 secondary structural elements and XL5 chemical groups are labeled with the two chiral centers indicated by an asterisk (*). FIG. 3E, Enlarged view highlighting interactions between hRpn13 M31, V85 and V93 with XL5 H13 and H19 as well as hRpn13 V38 and P89 with the XL5 central benzene. A weak hydrogen bond is formed between the hRpn13 S90 hydroxy group and XL5 cyanide group (line labeled with “3.3”). Key interactions are highlighted with lines including distances (A) for XL5 hydrogen or cyanide nitrogen atoms with hRpn13 carbon atoms.

FIGS. 4A-4E show chemical basis of hRpn13 targeting by XL5. FIGS. 4A-4E, Ribbon diagram structures of hRpn13 Pru ligated to XL5 to highlight key interactions, which are indicated by grey lines with distances (A) included. FIG. 4A, Comparison of XL5-ligated and free hRpn13 Pru (PDB 5IRS) structures with an expansion (dashed rectangles) in the right panel and hRpn13 W108 included. FIGS. 4B-4C, Structural comparison of XL5-ligated hRpn13 and hRpn2-bound hRpn13 (PDB: 6CO4) with hRpn2 colored as in FIG. 1C. FIG. 4D, hRpn13 M31, L33, V38, and V93 interact with the XL5 benzoic acid group. FIG. 4E, XL5 4-methyl benzamide interacts with hRpn13 V38, T39 and P40.

FIGS. 5A-5G show XL5-PROTAC compounds target a truncated hRpn13 product in MM cells. FIG. 5A, Chemical structures of XL5 (left-hand side of molecules)-PROTACs (VHL, right-hand side in structures with “VHL” in name; CRBN, right-hand side in structure with “CRBN” in name, IAP, right-hand side in structure with “lAP” in name). FIG. 5B, RPMI 8226 cells were treated with the indicated concentration of XL5, XL5-VHL, XL5-VHL-2, XL5-CRBN, XL5-IAP, VHL-Ac, thalidomide, or IAP-Bz for 48 hours and cell metabolism measured by an MTT assay (mean±SD). Viability is plotted as (I₅₇₀)ₛₐₘₚₗₑ/(I₅₇₀)_{control}×100(%). FIG. 5C, Immunoblot of whole cell extract from RPMI 8226 cells treated for 24 hours with 40 μM XL5 or XL5-PROTAC or DMSO (vehicle control) detecting hRpn13 (1s or 3 min exposure) or β-actin. FIG. 5D, Illustration of hRpn13-encoding ADRM1 displaying exons, hRpn13 Pru and DEUBAD, binding sites for ubiquitin (Ub), hRpn2, UCHL5, and anti-hRpn13 antibody used, and the trRpn13 protein expressed in HCT116 trRpn13 cells. FIG. 5E, Immunoblots of GST-hRpn2 (940-953) or GST (control) pulldown experiments (left) and anti-hRpt3 or IgG (control) antibody immunoprecipitates from RPMI 8226 cell lysates (middle) or of whole cell extract from indicated cells (right). A faster migrating hRpn13 product is indicated (arrow) in FIGS. 5C and 5E that is up-regulated in RPMI 8226 cells. FIG. 5F, Representation of increased hRpn13 C₈₈ accessibility following DEUBAD deletion (solid black line, PDB 5IRS) compared to full length hRpn13 (dashed grey line, PDB 2KR0). FIG. 5G, Cartoon depicting the proteasome CP (partial view, dark gray) and RP (not colored) with hRpn2 (labeled) bound to full length (left) or truncated (right) hRpn13 (labeled). DEUBAD inclusion allows UCHL5 (labeled) recruitment.

FIG. 6A shows immunoblot of whole-cell extract from RPMI 8226 WT, trRpn13-MM1, or trRpn13-MM2 cells probing hRpn13 (1 s and 20 min exposure) or β-actin. FIG. 6B shows Sanger sequencing analyses of hRpn13 cDNA from RPMI 8226 WT, trRpn13-MM1, ortrRpn13-MM2 cells denoting the location of the two sgRNAs (arrow) on hRpn13-encoding gene ADRM1 Exon 2 with cDNA sequence (CDS) labeled. Allele is abbreviated as “A”. FIG. 6C shows RPMI 8226 WT (top line), trRpn13-MM1 (middle line) or trRpn13-MM2 (bottom line) cells were treated with the indicated concentrations of XL5-VHL-2 for 48 h and cell metabolism measured by an MTT assay; data represent mean±SD of n=6 biological replicates. Viability is calculated as (λ₅₇₀)ₛₐₘₚₗₑ/(λ₅₇₀)^{control}×100(%). FIG. 6D shows immunoblots of whole cell lysate from RPMI 8226 WT, trRpn13-MM1, or trRpn13-MM2 cells treated for 24 h with 40 μM XL5-VHL-2 with comparison to DMSO (vehicle control) immunoprobing for cleaved caspase-9 (top panel), hRpn13 (two middle panels with 1 min or 10 min exposure), or β-actin (as a loading control, bottom panel). A black asterisk indicates cleaved caspase-9 in the 1-min immunoblot for hRpn13, as hRpn13 was probed following cleaved caspase-9 and without stripping the membrane.

FIG. 7A shows lysates from RPMI 8226 WT cells treated with indicated concentration of XL5-VHL-2 or DMSO (control) for 24 h were immunoprobed for cleaved caspase-9, hRpn13 (1 s or 40 s exposure), or β-actin (as a loading control). A black asterisk indicates cleaved caspase-9 in the 40 s immunoblot for hRpn13, as hRpn13 was probed following cleaved caspase-9 and without stripping the membrane (top panel). FIG. 7B shows Immunoblots of whole-cell extract from RPMI 8226 WT cells treated for the indicated hours with 40 μM XL5-VHL-2 or DMSO (0 h, vehicle control) detecting hRpn13 or β-actin. Percentage (%) is calculated as the ratio of intensities for hRpn13^{Pru} normalized to β-actin (lhRpn13^{Pru}/lβ-actin)sample divided by that of DMSO-treated cells and multiplied by 100. Percentage (%) derived from left (FIG. 7A) or top (FIG. 7B) panel immunoblots were plotted against XL5-VHL-2 concentration (FIG. 7A, μM) or time (FIG. 7B, hours) and fit by using the equation [Inhibitor] vs. normalized response−Variable slope (FIG. 7A) and One phase decay (FIG. 7B) in GraphPad Prism8. Half degrading concentration (DC₅₀, FIG. 7A), maximal degradation (Dₘₐₓ, FIG. 7A), and half-life (t_{1/2}, FIG. 7B) values are included.

FIG. 8A shows LC-MS analysis of GST-hRpn2 (940-953) (control, left panel) or GST-hRpn2 (940-953)-pulldown sample from lysates of RPMI 8226 WT cells (right panel). The mass spectra (upper panel) were deconvoluted from the UV peak (lower panel) indicated with a black arrow. FIG. 8B shows lysates from Hs27, SK-OV-3, MM.1S, NCI-H929, or RPMI 8226 WT cells were immunoprobed for hRpn13 and β-actin as indicated. FIG. 8C shows MM1.S cells were treated with 2.5 or 5 μM of XL5-VHL-2 for 48 h and cell metabolism measured by an MTT assay; data represent mean±SD of n=6 biological replicates. Viability is calculated as (λ₅₇₀)ₛₐₘₚₗₑ/(λ₅₇₀)_{control}×100(%). FIG. 8D shows lysates from RPMI 8226 WT and trRpn13-MM2 cells transfected for 48 h with empty vector (EV) or plasmids expressing FLAG-hRpn13 full length or FLAG-hRpn131-279 proteins were treated for 24 h with 40 μM XL5-VHL-2 or DMSO (vehicle control) and immunoprobed as indicated with antibodies against hRpn13, cleaved caspase-9, and β-actin. Immunoprobing for cleaved caspase-9 and hRpn13 was done separately with re-probing for β-actin. FIG. 8E shows a volcano plot displaying proteomic changes caused by XL5-VHL-2 treatment determined by quantitative TMT proteomics analysis performed on lysates from RPMI 8226 trRpn13-MM2 cells treated for 24 h with DMSO (control) or 40 μM XL5-VHL-2 in triplicate. p value was calculated by two-tailed two-sample equal variance t test. A dashed line indicates the value −log₁₀0.05.

FIG. 9 shows tumor xenograft lysates from myeloma, prostate, and pancreatic adenocarcinoma models contain hRpn13-Pru.

FIG. 10 shows hRpn13-Pru PROTACs with two different linkers differentially induce apoptosis in WT RPMI 8226 cells, but not when the hRpn13 Pru domain is deleted by gene editing (trRpn13-MM2). The triazole linker in XL5-VHL-2 showed poorer induction of cleaved caspase-9 and less hRpn13-Pru loss. RPMI 8226 WT or trRpn13-MM2 (MM2) cells were treated with 40 μM XL5-VHL-2, 40 μM XL5-VHL-3 or DMSO (vehicle control) and the whole cell extract immunoprobed for hRpn13 or cleaved caspase-9 with β-actin as a loading control. A black asterisk indicates cleaved caspase-9 in the 2-min immunoblot for hRpn13, as hRpn13 was probed following cleaved caspase-9 and without stripping the membrane.

FIGS. 11A-11C show hRpn13-Pru PROTACs with different linkers restrict cell viability in an hRpn13 Pru-dependent manner. RPMI 8226 WT (darker shades) or trRpn13-MM2 (lighter shades) cells were treated with the indicated concentration of XL5-VHL-2 (FIG. 11A), XL5-VHL-3 (FIG. 11A), XL5-VHL-4 (FIG. 11B) or XL5-VHL-5 (FIG. 11C) for 48 hours and cell metabolism measured by an MTT assay; data represent mean±SD of n=6 biological replicates. Viability is calculated as (λ₅₇₀)ₛₐₘₚₗₑ/(λ₅₇₀)_{control}×100(%).

FIG. 12 shows XL5-S2 (left) induces apoptosis in WT RPMI 8226 cells, but not when the hRpn13 Pru domain is deleted by gene editing (trRpn13-MM2, right). RPMI 8226 WT or trRpn13-MM2 cells were treated with 20 μM XL5-S2 or DMSO (vehicle control) and the whole cell extract immunoprobed for hRpn13 or cleaved caspase-9, with β-actin as a loading control. A black asterisk indicates cleaved caspase-9 in the 5-min immunoblot for hRpn13, as hRpn13 was probed following cleaved caspase-9 and without stripping the membrane.

FIGS. 13A-13B show the structure of XL5-S2-ligated hRpn13 Pru in complex with ubiquitin. (FIG. 13A) A ribbon diagram is shown with secondary structural elements labeled for hRpn13 Pru (middle ribbon diagram) and ubiquitin (bottom ribbon diagram). The 2Fo−Fc difference electron density map of XL5-S2 (stick diagram) is shown contoured at 1.0a. (FIG. 13B) Interactions between hRpn13 Pru and XL5-S2 are displayed showing polar and hydrophobic interactions for expanded regions of XL5-S2 methoxybenzamide, benzene and indolin-2-one rings.

FIGS. 14A-14C show that degradation of hRpn13^{Pru} by XL5-VHL-2 is mediated through VHL. FIG. 14A: Chemical structure of a XL5-VHL-2 Epimer with a VHL-inactive degrader module due to altered stereochemistry. FIG. 14B: Immunoblots of whole cell extract from RPMI 8226 WT cells treated for 24 hours with 40 μM XL5-VHL-2 or XL5-VHL-2 Epimer compared to DMSO (vehicle control) detecting cleaved caspase-9, hRpn13 (1-second and 2-minute exposure) or β-actin. A black asterisk indicates cleaved caspase-9 in the 2-minute immunoblot for hRpn13, as hRpn13 was probed following cleaved caspase-9 and without stripping the membrane. FIG. 14C: Immunoblots of whole cell extract from RPMI 8226 WT cells treated for 24 hours with 40 μM XL5-VHL-2 with or without 40 μM VHL-ligand or DMSO (control) detecting hRpn13 or β-actin.

FIG. 15 shows the proteasome is responsible for generating hRpn13-Pru. Lysates from RPMI 8226 WT cells treated for 24 hours with 100 nM carfilzomib or DMSO were immunoprobed for hRpn13 with 1 or 30 minute exposure times and β-actin, as indicated.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “an excipient,” “a PROTAC,” “an hRpn13 binder,” or “an hRpn13 inhibitor,” include, but are not limited to, mixtures or combinations of two or more such excipients, PROTACs, hRpn13 binders, or hRpn13 inhibitors, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, “IC₅₀,” is intended to refer to the concentration of a substance (e.g., a compound or a drug) that is required for 50% inhibition of a biological process, or component of a process. For example, IC₅₀ refers to the half maximal (50%) inhibitory concentration (IC) of a substance as determined in a suitable assay. For example, an IC₅₀ for hRpn13 can be determined in an in vitro or cell-based assay system. Frequently, receptor assays make use of a suitable cell-line, e.g. a cell line that either expresses endogenously a target of interest, or has been transfected with a suitable expression vector that directs expression of a recombinant form of the target. For example, the IC₅₀ for a compound disclosed herein can be determined using mammalian cells transfected with hRpn13.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

The position of a substituent can be defined relative to the positions of other substituents in an aromatic ring. For example, as shown below in relationship to the “R” group, a second substituent can be “ortho,” “para,” or “meta” to the R group, meaning that the second substituent is bonded to a carbon labeled ortho, para, or meta as indicated below. Combinations of ortho, para, and meta substituents relative to a given group or substituent are also envisioned and should be considered to be disclosed.

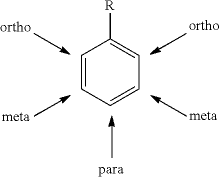

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkanediyl” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂— are non-limiting examples of alkanediyl groups.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or -OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl. Fused aryl groups including, but not limited to, indene and naphthalene groups are also contemplated.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) and —N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ or -(A¹O(O)C-A²-OC(O))ₐ, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen” or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxy” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula -SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A'S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A'S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” . . . “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘}₂ may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{●} include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

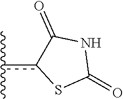

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine and chlorine, such as ²H, ³H, ¹³C, ¹⁴C ¹⁵N, ¹⁸O, ¹⁷O ³⁵S, ¹⁸F, and ³⁶Cl, respectively.

Compounds further comprise prodrugs thereof and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

It is also appreciated that certain compounds described herein can be present as an equilibrium of tautomers. For example, ketones with an α-hydrogen can exist in an equilibrium of the keto form and the enol form.

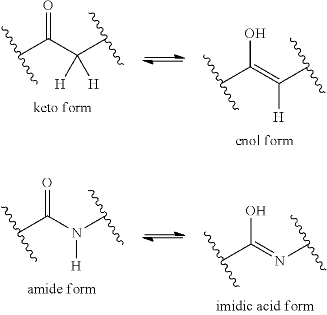 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

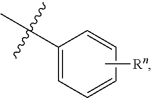

which is understood to be equivalent to a formula:

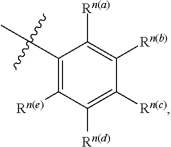

wherein n is typically an integer. That is, Rⁿ is understood to represent five independent substituents, R^{n(a)}, R^{n(b)}, R^{n(c)}, R^{n(d)}, and R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the term “effective amount” refers to an amount that is sufficient to achieve the desired modification of a physical property of the composition or material. For example, an “effective amount” of an hRpn13 binder refers to an amount that is sufficient to achieve the desired improvement in the property modulated by the formulation component, e.g. achieving the desired level of inhibition of aberrant hRpn13 activity, or, in the case of the PROTACs disclosed herein, achieving the desired level of ubiquitination and/or degradation of hRpn13 and/or hRpn13-Pru. The specific level in terms of wt % in a composition required as an effective amount will depend upon a variety of factors including the amount and type of compound, type of cell or tissue, co-administration of additional therapies, and type of cancer or other disorder that is to be treated.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

### Compounds and Methods of Making and Using the Compounds

In one aspect, disclosed herein is a compound having a structure according to Formula I:

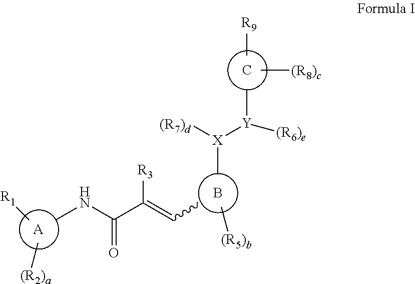

wherein A, B, and C independently are an aryl or heteroaryl ring having 5-10 members;
wherein X and Y independently are carbon, oxygen, nitrogen, sulfur, a carbonyl group, or a sulfonyl group;
   wherein each instance of R₆ and R₇ is absent or independently is hydrogen, halogen, hydroxyl, trifluoromethyl, C1-C6 alkylamino, C1-C6 alkoxy, substituted or unsubstituted C1-C6 alkyl, a substituted or unsubstituted phenyl group, or Formula II;

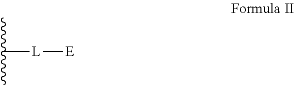

wherein L is a linker moiety;
wherein E is selected from an E3 ubiquitin ligase targeting moiety, a bridging molecule to a ubiquitin E3 ligase complex, an E2 ubiquitin conjugating enzyme targeting molecule, an autophagy-targeting chimera (AUTAC), or a proteasome subunit targeting molecule;
wherein when at least one R₇ is present, d is 1 or 2; and
wherein when at least one R₆ is present, e is 1 or 2;
wherein R₁ is —SO₂NH₂, a carboxylic acid group, fluorine, a trifluoromethyl group, or a tetrazole;
wherein each instance of R₂ independently is hydrogen, halogen, hydroxyl, trifluoromethyl, C1-C6 alkylamino, C1-C6 alkoxy, or substituted or unsubstituted C1-C6 alkyl, and a is from 1 to 4;
wherein R₃ is a cyano group, —S(═O)₂—R₄, or —C(═O)—R₄, —C(═O)—OR₄, —C(═O)—N—R₄,R₄′, or —S(═O)₂—NH₂;
   wherein R₄ and R₄′ are hydrogen, halogen, hydroxyl, trifluoromethyl, C1-C6 alkylamino, C1-C6 alkoxy, substituted or unsubstituted C1-C6 alkyl, or a substituted or unsubstituted phenyl group;
wherein each instance of R₅ independently can be hydrogen, halogen, hydroxyl, trifluoromethyl, C1-C6 alkylamino, C1-C6 alkoxy, substituted or unsubstituted C1-C6 alkyl, a substituted or unsubstituted phenyl group, or Formula II, and wherein b is from 1 to 5;
wherein each instance of R₆ independently can be hydrogen, halogen, hydroxyl, trifluoromethyl, C1-C6 alkylamino, C1-C6 alkoxy, or substituted or unsubstituted C1-C6 alkyl; and wherein c is from 1 to 5; and
wherein R₉ can be hydrogen, halogen, hydroxyl, trifluoromethyl, substituted or unsubstituted C1-C6 alkylamino, substituted or unsubstituted C1-C6 alkyl, or Formula II.

In some aspects, E can further be a proteasome subunit-targeting molecule, such as an inhibitor of Rpn11. In one aspect, the Rpn11 inhibitor can be capzimin or a derivative thereof.

In some aspects, the compound of Formula I is not XL5 or XL23:

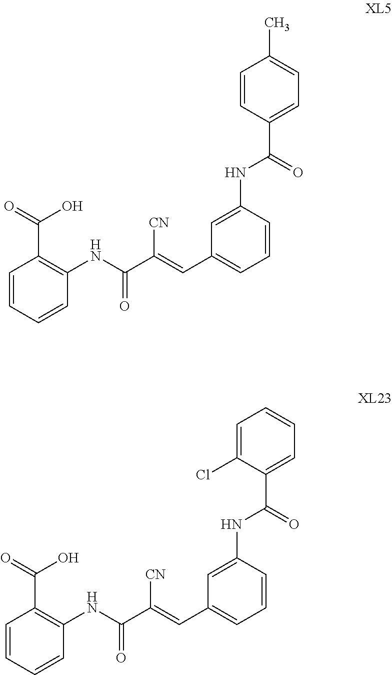 [SMILES: Cc1ccc(C(=O)Nc2cccc(/C=C(\C#N)C(=O)Nc3ccccc3C(=O)O)c2)cc1 ; N#C/C(=C\c1cccc(NC(=O)c2ccccc2Cl)c1)C(=O)Nc1ccccc1C(=O)O]

In another aspect, the compound of Formula I can have a stereochemistry about a double bond such that the compound has Formula Ia or Formula Ib, or any combination thereof:

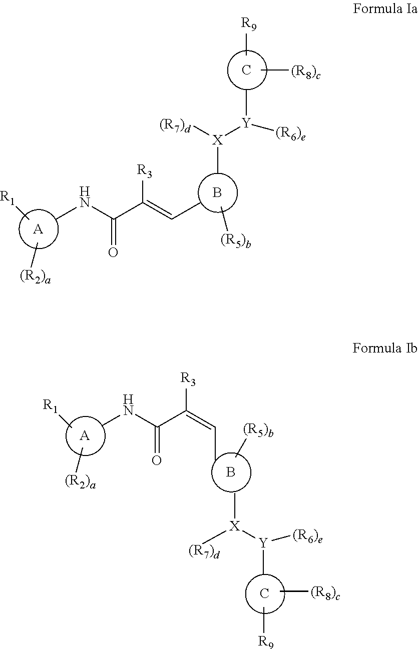

In certain aspects, A and B can be a substituted or unsubstituted phenyl group. In another aspect, C can be a substituted or unsubstituted phenyl or pyridyl group. In one aspect, X can be nitrogen. In another aspect, R₇ can be hydrogen and d can be 1. In any of these aspects, Y can be a carbonyl group and R₈ can be absent. In one aspect, R₁ can be —SO₂NH₂ or a carboxylic acid group.

In one aspect, each R₂ can independently be hydrogen, trifluoromethyl, methylamino, or methoxy, and a can be 4. In another aspect, R₃ can be a cyano group.

In one aspect, each R₅ can independently be hydrogen, trifluoromethyl, or methylamino, and b can be 4. In another aspect, each R₆ can independently be chloro, hydrogen, or hydroxyl, and c can be 4.

In still another aspect, each R₉ can independently be hydrogen, methyl, methylamino, trifluoromethyl, —NHCH₂COOH, or Formula II.

In some aspects, at least one of R₅, R₆, R₇, or R₉ can be formula II and L can be:

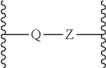

wherein Q is a triazole, an amide, a C1-C4 alkyl amide, a sulfonamide, or substituted or unsubstituted spirocyclic rings; and wherein Z is selected from an alkyl group, an alkylene group, a polyether group, or any combination thereof.

In one aspect, when Q includes substituted or unsubstituted spirocyclic rings, the substituted or unsubstituted spirocyclic rings can be selected from:

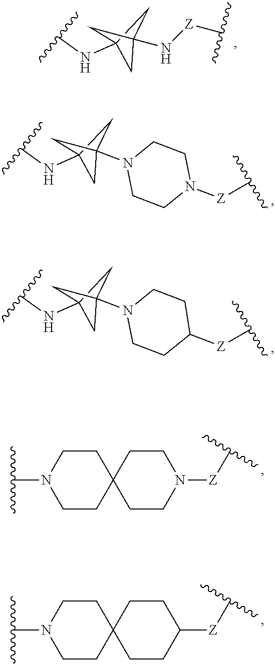

or any combination thereof.

In another aspect, Z can be

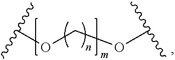

wherein n is 2 or 3 and wherein m is from 1 to 10; or

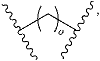

wherein o is from 0 to 10.

In some aspects, when R₉ is Formula II, L can be:

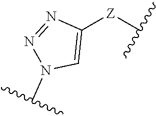

wherein Z can be an alkyl group, an alkylene group, a polyether group, or any combination thereof.

In one aspect, R₉ can be Formula II and L can be:

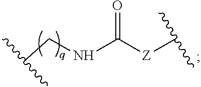

wherein q is 0 or 1;
and wherein Z is an alkyl group, an alkylene group, a polyether group, or any combination thereof.

In another aspect, R₉ can be Formula II and L can be:

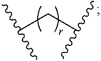

and wherein r is from 1 to 5.

In other aspects, L can be represented by one of the following structures:

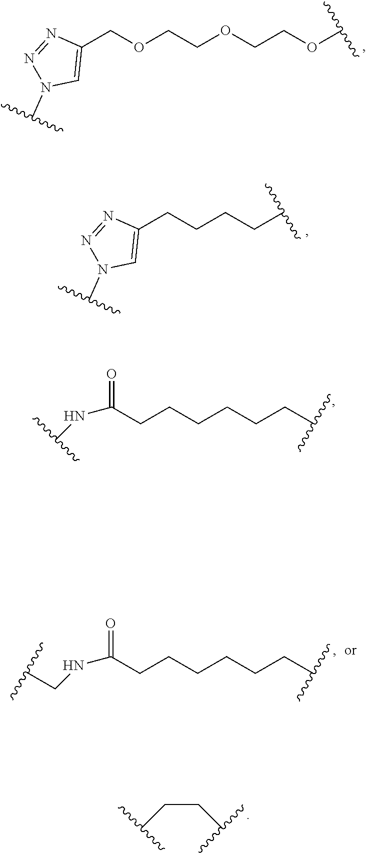

In one aspect, the compound can be represented by a structure of Formula III:

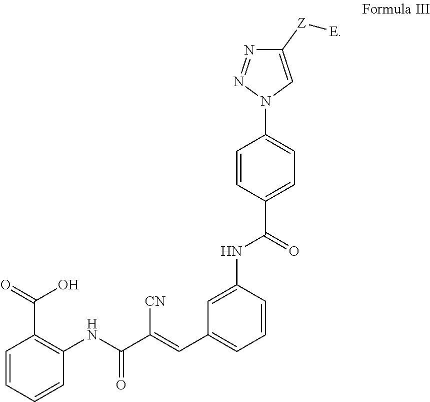

In one aspect, E can be a cereblon-targeting molecule, a von Hippel-Lindau targeting molecule, an IAP E3 ligase targeting molecule, an MDMs-targeting E3 ligase, an autophagy targeting chimera (AUTAC), or an Rpn11-targeting molecule. In another aspect, the cereblon-targeting molecule can be thalidomide, lenalidomide, pomalidomide, iberdomide, or apremilast. In one aspect, E is an AUTAC represented by a structure:

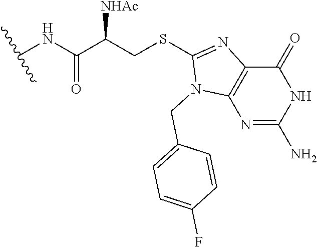 [SMILES: *NC(=O)[C@H](CSc1nc2c(=O)[nH]c(N)nc2n1Cc1ccc(F)cc1)NC(C)=O]

In one aspect, R₉ can be formula II and E can be selected from at least the following:

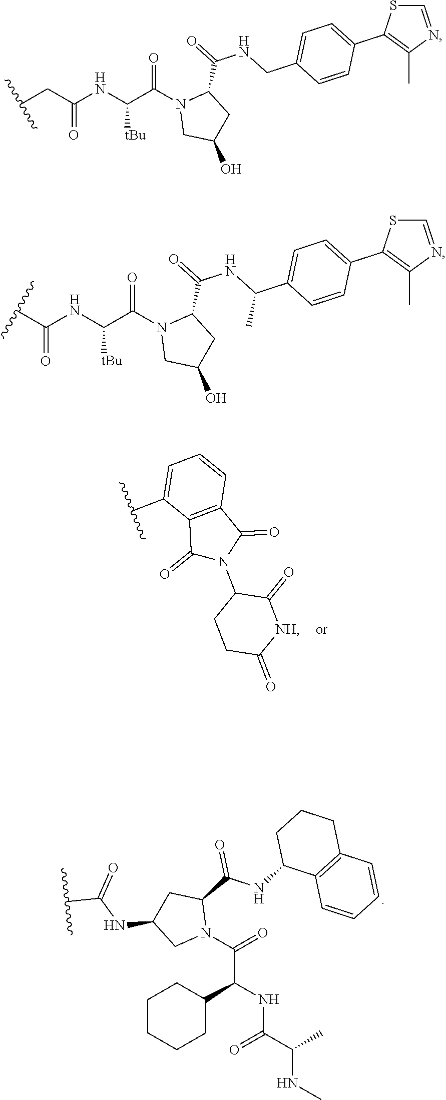 [SMILES: *CC(=O)N[C@H](C(=O)N1C[C@H](O)C[C@H]1C(=O)NCc1ccc(-c2scnc2C)cc1)C(C)(C)C ; *C(=O)N[C@H](C(=O)N1C[C@H](O)C[C@H]1C(=O)N[C@@H](C)c1ccc(-c2scnc2C)cc1)C(C)(C)C ; *c1cccc2c1C(=O)N(C1CCC(=O)NC1=O)C2=O ; *C(=O)N[C@H]1C[C@@H](C(=O)N[C@@H]2CCCc3ccccc32)N(C(=O)[C@@H](NC(=O)[C@H](C)NC)C2CCCCC2)C1]

In still another aspect, the disclosed compound can have a structure represented by a formula:

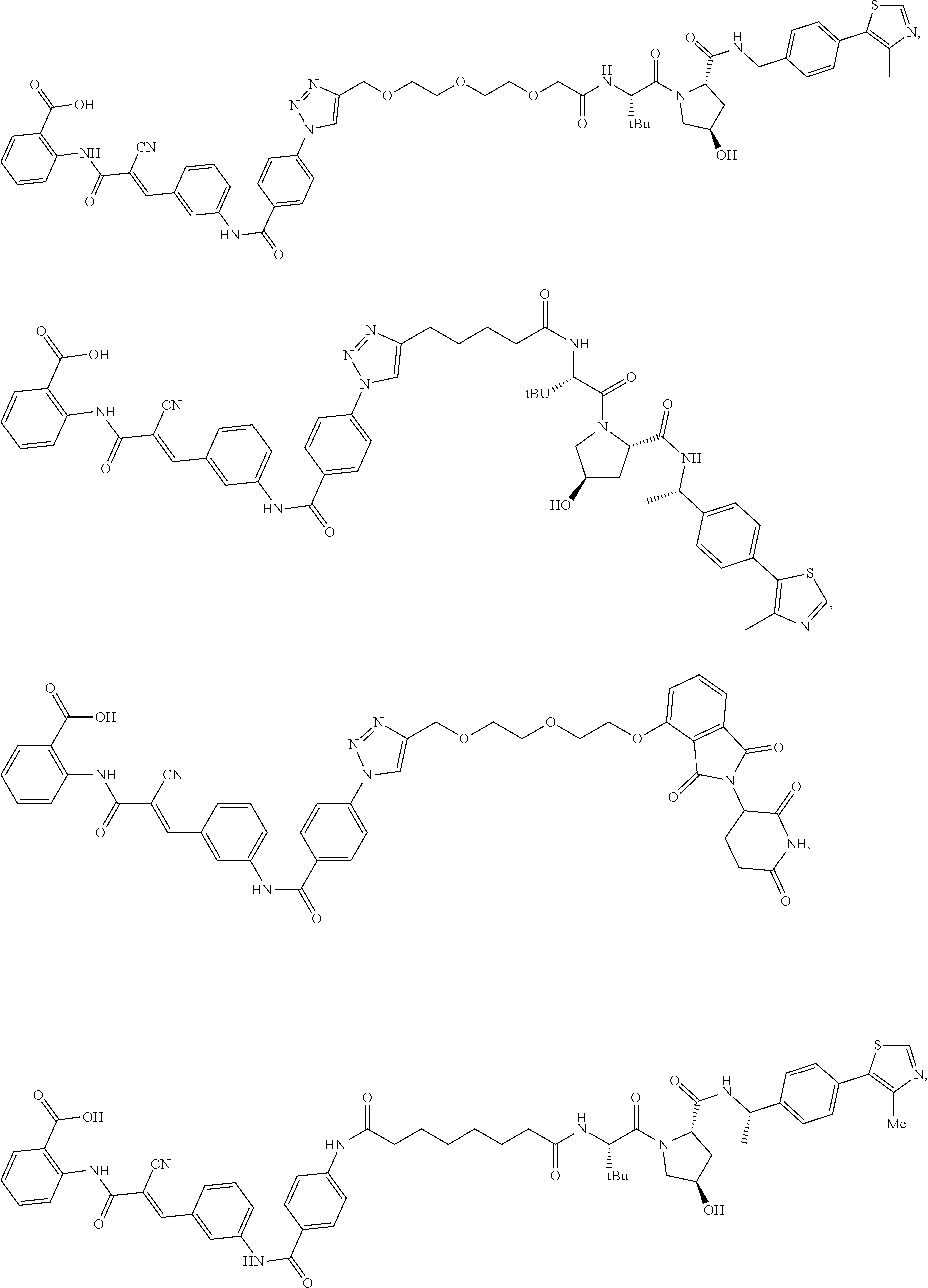

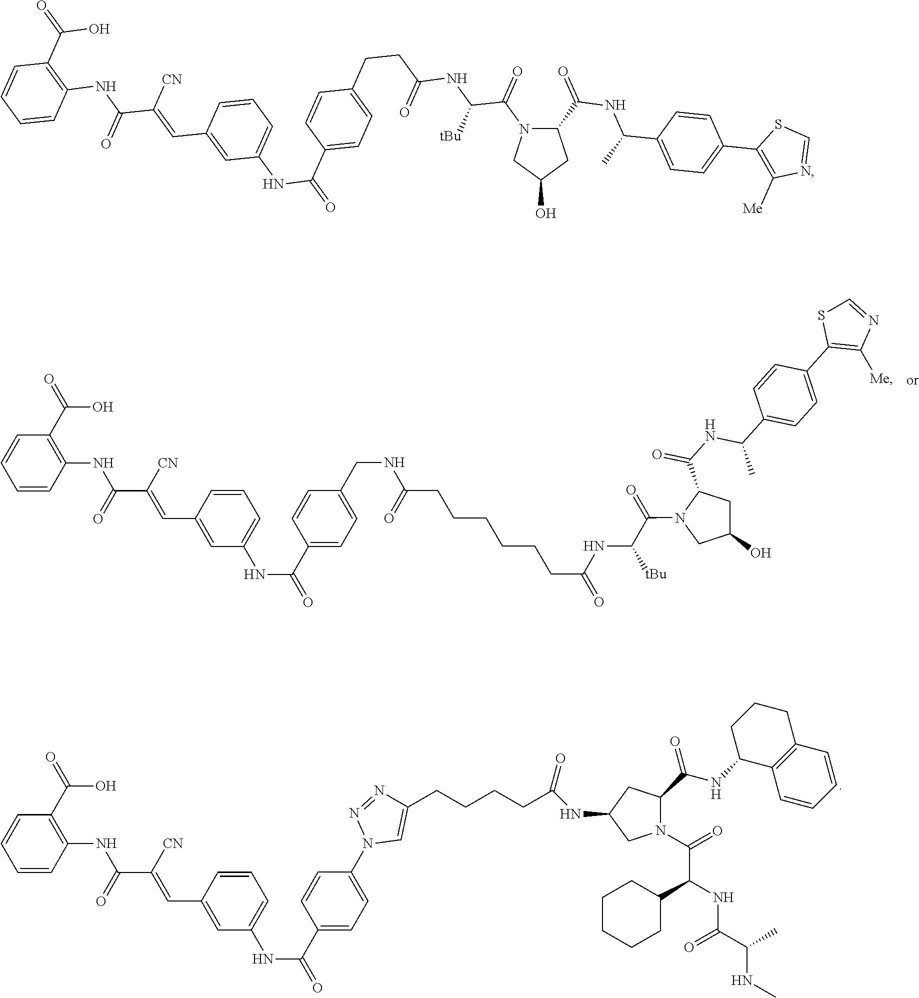 [SMILES: Cc1ncsc1-c1ccc([C@H](C)NC(=O)[C@@H]2C[C@@H](O)CN2C(=O)[C@@H](NC(=O)CCc2ccc(C(=O)Nc3cccc(/C=C(\C#N)C(=O)Nc4ccccc4C(=O)O)c3)cc2)C(C)(C)C)cc1 ; Cc1ncsc1-c1ccc([C@H](C)NC(=O)[C@@H]2C[C@@H](O)CN2C(=O)[C@@H](NC(=O)CCCCCCC(=O)NCc2ccc(C(=O)Nc3cccc(/C=C(\C#N)C(=O)Nc4ccccc4C(=O)O)c3)cc2)C(C)(C)C)cc1 ; CN[C@@H](C)C(=O)N[C@H](C(=O)N1C[C@@H](NC(=O)CCCCc2cn(-c3ccc(C(=O)Nc4cccc(/C=C(\C#N)C(=O)Nc5ccccc5C(=O)O)c4)cc3)nn2)C[C@H]1C(=O)N[C@@H]1CCCc2ccccc21)C1CCCCC1]

In another aspect, the disclosed compound can have a structure represented by a formula:

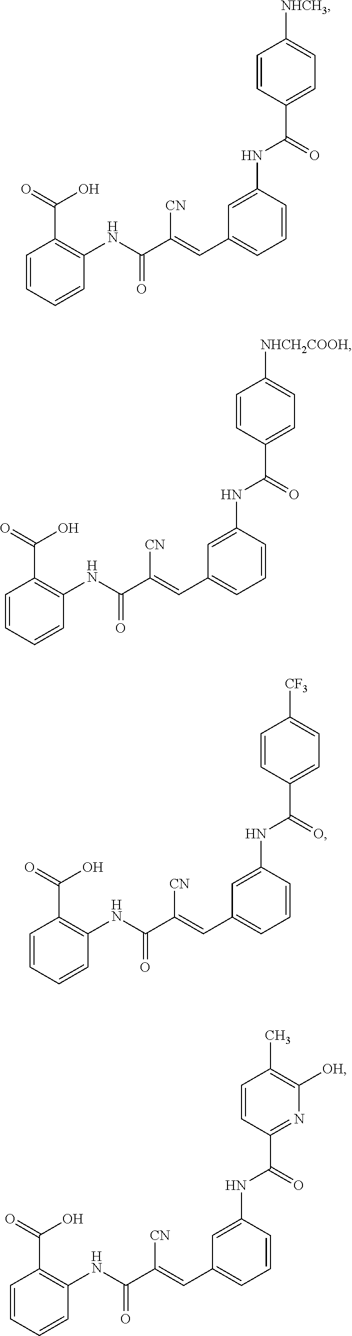

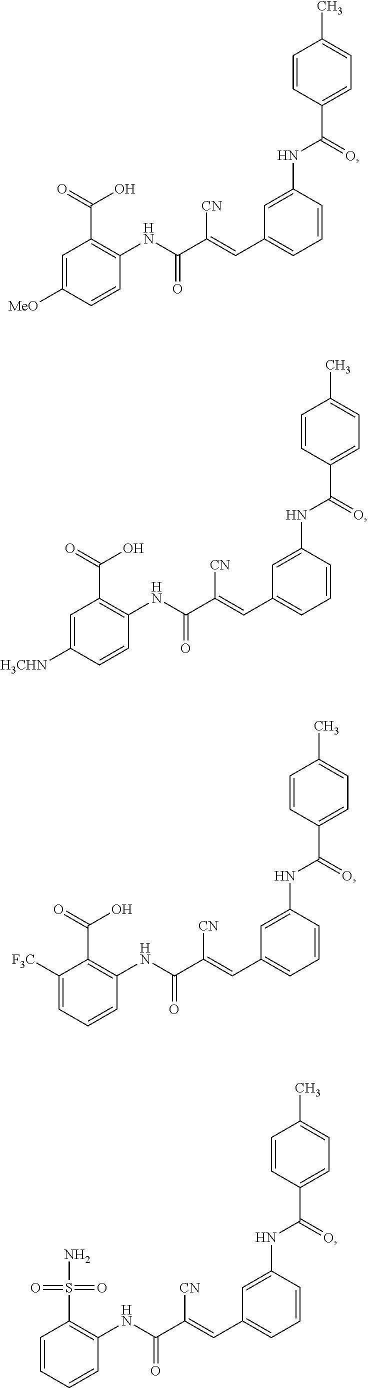

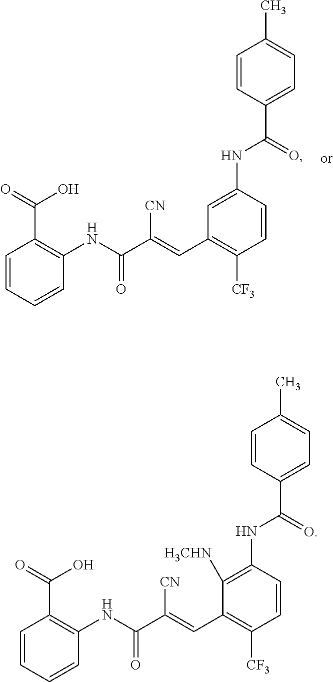

In any of these aspects, the compound can include a fluorescent label such as, for example, Cy5, Cy7, Alexafluor, BODIPY, rhodamine, or any combination thereof.

In another aspect, provided herein is a compound having a structure of Formula IV:

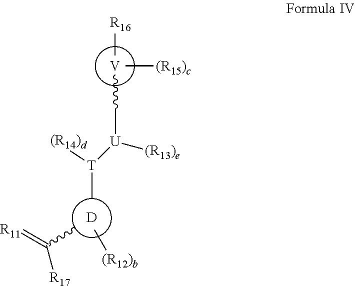

wherein D and V independently comprise an aryl or heteroaryl ring having 5-10 members;
wherein T and U independently comprise carbon, oxygen, nitrogen, sulfur, a carbonyl group, or a sulfonyl group;
   wherein each instance of R₁₃ and R₁₄ is absent or independently comprises hydrogen, halogen, hydroxyl, trifluoromethyl, C1-C6 alkylamino, C1-C6 alkoxy, substituted or unsubstituted C1-C6 alkyl, a substituted or unsubstituted phenyl group, or Formula II;

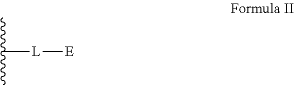

wherein L comprises a linker moiety;
wherein E comprises an E3 ubiquitin ligase targeting moiety, a bridging molecule to a ubiquitin E3 ligase complex, an E2 ubiquitin conjugating enzyme targeting molecule, an autophagy-targeting chimera, or a proteasome subunit targeting molecule;
wherein when at least one R₁₄ is present, d is 1 or 2; and
wherein when at least one R₁₃ is present, e is 1 or 2;
wherein R₁₁ comprises a substituted or unsubstituted bicyclic ring or a two-ring system, the bicyclic ring or two-ring system having 9 or 10 members with a carbonyl group at an ortho position to the alkene;
wherein each instance of R₁₂ independently comprises hydrogen, halogen, hydroxyl, trifluoromethyl, C1-C6 alkylamino, C1-C6 alkoxy, substituted or unsubstituted C1-C6 alkyl, a substituted or unsubstituted phenyl group, or Formula II, and wherein b is from 1 to 5;
wherein each instance of R₁₅ independently comprises hydrogen, halogen, hydroxyl, trifluoromethyl, C1-C6 alkylamino, C1-C6 alkoxy, substituted or unsubstituted C1-C6 alkyl, or Formula II; and wherein c is from 1 to 5;
wherein R₁₆ comprises hydrogen, halogen, hydroxyl, trifluoromethyl, substituted or unsubstituted C1-C6 alkylamino, substituted or unsubstituted C1-C6 alkyl, an azide group, or Formula II; and
wherein R₁₇ is hydrogen or C1-C6 cyclic or linear alkyl.

In another aspect, R₁₁ can be selected from:

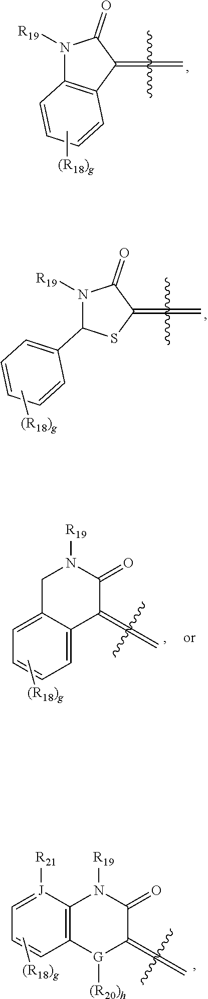

wherein G is C or S;
   wherein, when G is C, h is 2 or wherein, when G is S, h is 0;
   wherein each R₂₀ is independently selected from H, C1-C4 alkyl, or C3-C6 cycloalkyl; wherein J is N or C;
   wherein, when J is N, R₂₁ is absent or, wherein, when J is C, R₂₁ is H;
wherein R₁₉ is selected from H, substituted or unsubstituted C1-C4 alkyl, or substituted or unsubstituted C3-C6 cycloalkyl or heterocycloalkyl; and
wherein each R₁₈ is independently selected from H, halogen, substituted or unsubstituted C1-C4 alkyl, C1-C6 alkoxy, substituted or unsubstituted C3-C6 cycloalkyl or heterocycloalkyl, —COOH, —OCF₃, —CF₃, or CN, and wherein g is from 1 to 5.

In a further aspect, R₁₉ can be methyl, cyclopropyl, H, or

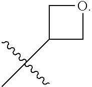 [SMILES: *C1COC1]

In another aspect, R₁₈ can be selected from H, —COOH, —OCF₃, —CF₃, CN, methyl, cyclopropyl, or

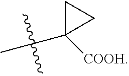 [SMILES: *C1(C(=O)O)CC1]

In an aspect, T can be nitrogen, U can be carbonyl, and R₁₃ and R₁₄ can be absent. In another aspect, D can be phenyl. In still another aspect, V can be phenyl. In one aspect, R₁₅ can be C1-C6 alkoxy. In one aspect, R₁₆ can be Formula II. In one aspect, R₁₅ can be C1-C6 alkoxy and R₁₆ can be Formula II.

In one aspect, disclosed herein is a compound having the following structure:

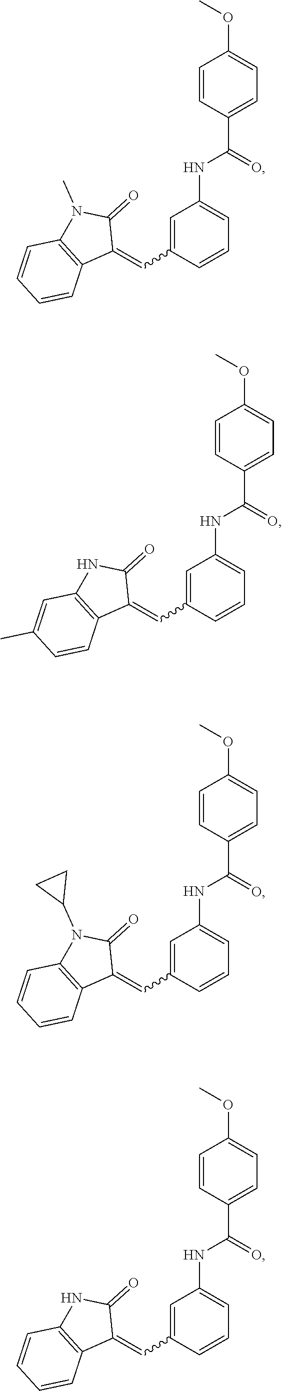 [SMILES: COc1ccc(C(=O)Nc2cccc(C=C3C(=O)N(C)c4ccccc43)c2)cc1 ; COc1ccc(C(=O)Nc2cccc(C=C3C(=O)Nc4cc(C)ccc43)c2)cc1 ; COc1ccc(C(=O)Nc2cccc(C=C3C(=O)N(C4CC4)c4ccccc43)c2)cc1 ; COc1ccc(C(=O)Nc2cccc(C=C3C(=O)Nc4ccccc43)c2)cc1]

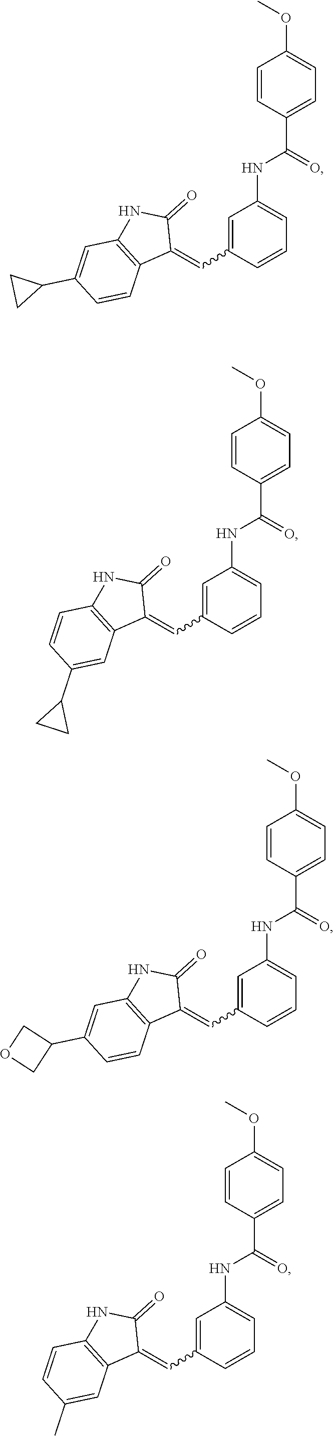 [SMILES: COc1ccc(C(=O)Nc2cccc(C=C3C(=O)Nc4cc(C5CC5)ccc43)c2)cc1 ; COc1ccc(C(=O)Nc2cccc(C=C3C(=O)Nc4ccc(C5CC5)cc43)c2)cc1 ; COc1ccc(C(=O)Nc2cccc(C=C3C(=O)Nc4cc(C5COC5)ccc43)c2)cc1 ; COc1ccc(C(=O)Nc2cccc(C=C3C(=O)Nc4ccc(C)cc43)c2)cc1]

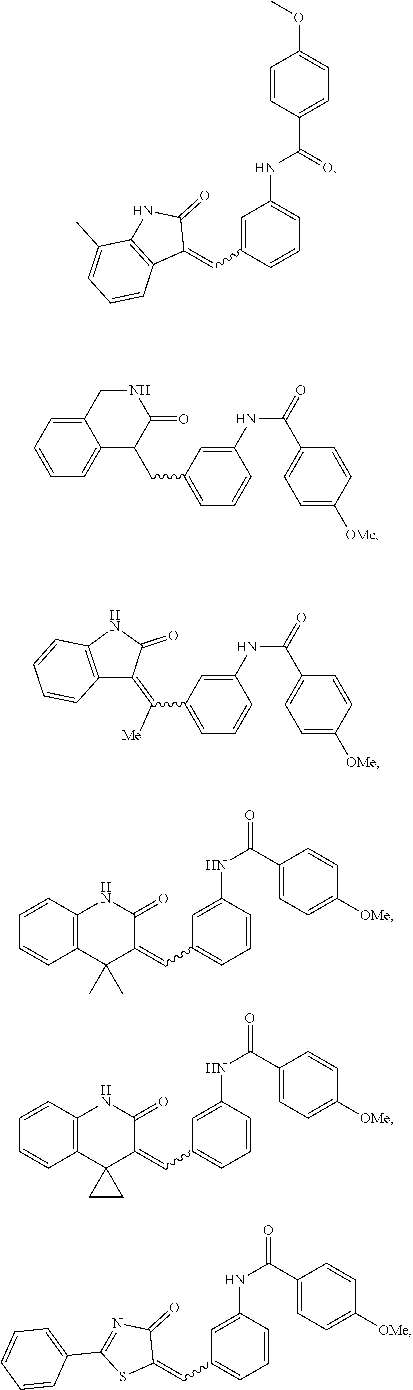 [SMILES: COc1ccc(C(=O)Nc2cccc(C=C3C(=O)Nc4c(C)cccc43)c2)cc1 ; COc1ccc(C(=O)Nc2cccc(CC3C(=O)NCc4ccccc43)c2)cc1 ; COc1ccc(C(=O)Nc2cccc(C(C)=C3C(=O)Nc4ccccc43)c2)cc1 ; COc1ccc(C(=O)Nc2cccc(C=C3C(=O)Nc4ccccc4C3(C)C)c2)cc1 ; COc1ccc(C(=O)Nc2cccc(C=C3C(=O)Nc4ccccc4C34CC4)c2)cc1 ; COc1ccc(C(=O)Nc2cccc(C=C3SC(c4ccccc4)=NC3=O)c2)cc1]

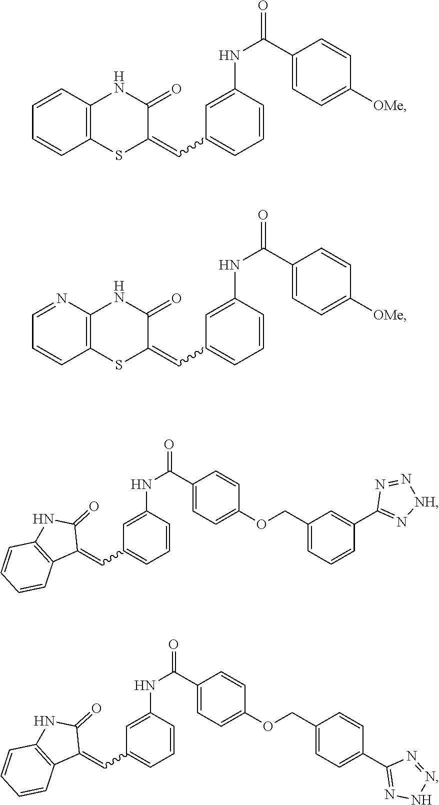 [SMILES: COc1ccc(C(=O)Nc2cccc(C=C3Sc4ccccc4NC3=O)c2)cc1 ; COc1ccc(C(=O)Nc2cccc(C=C3Sc4cccnc4NC3=O)c2)cc1 ; O=C1Nc2ccccc2C1=Cc1cccc(NC(=O)c2ccc(OCc3cccc(-c4nn[nH]n4)c3)cc2)c1 ; O=C1Nc2ccccc2C1=Cc1cccc(NC(=O)c2ccc(OCc3ccc(-c4nn[nH]n4)cc3)cc2)c1]

or any combination thereof.

In another aspect, disclosed herein is a compound having the structure:

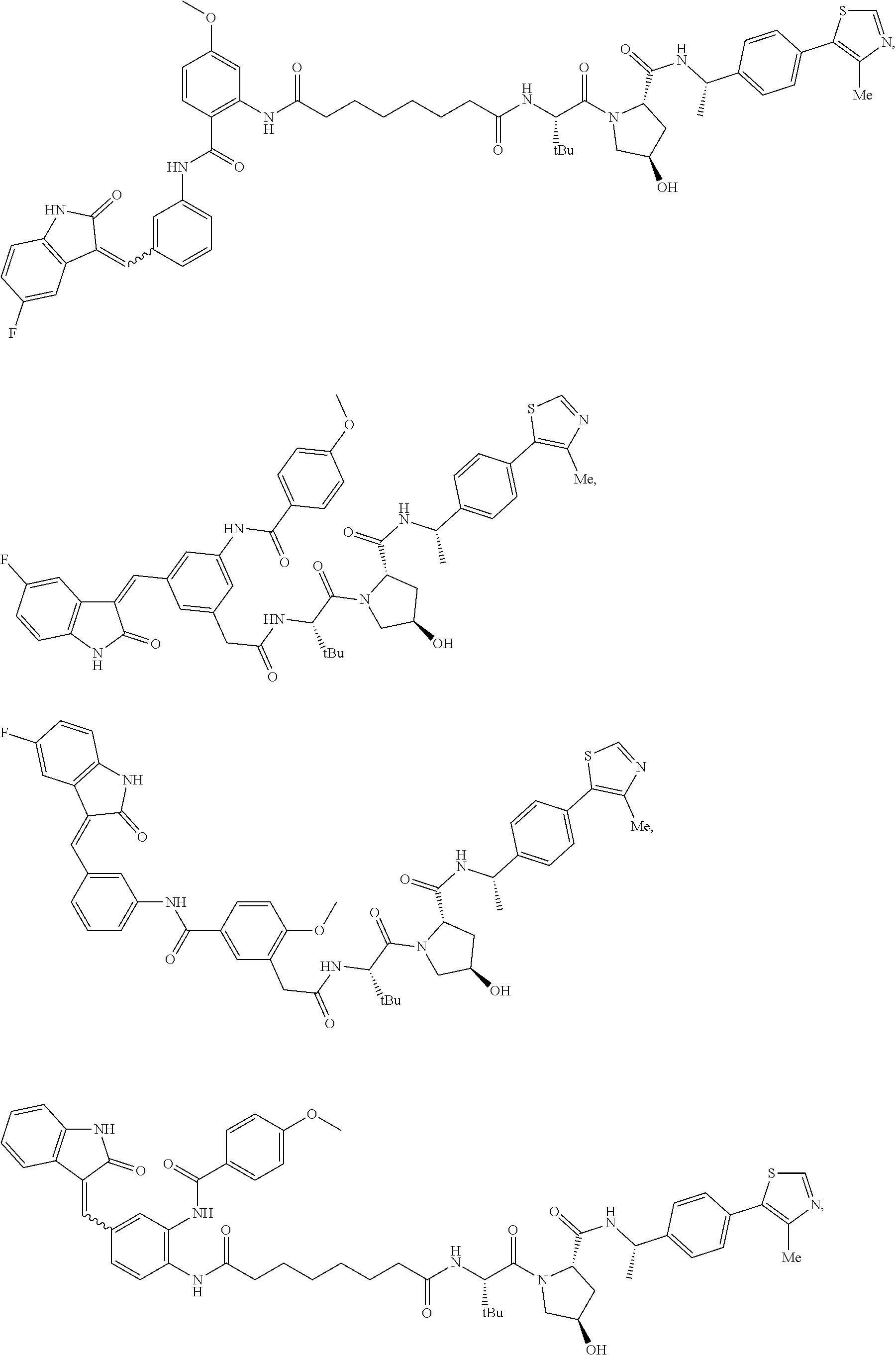

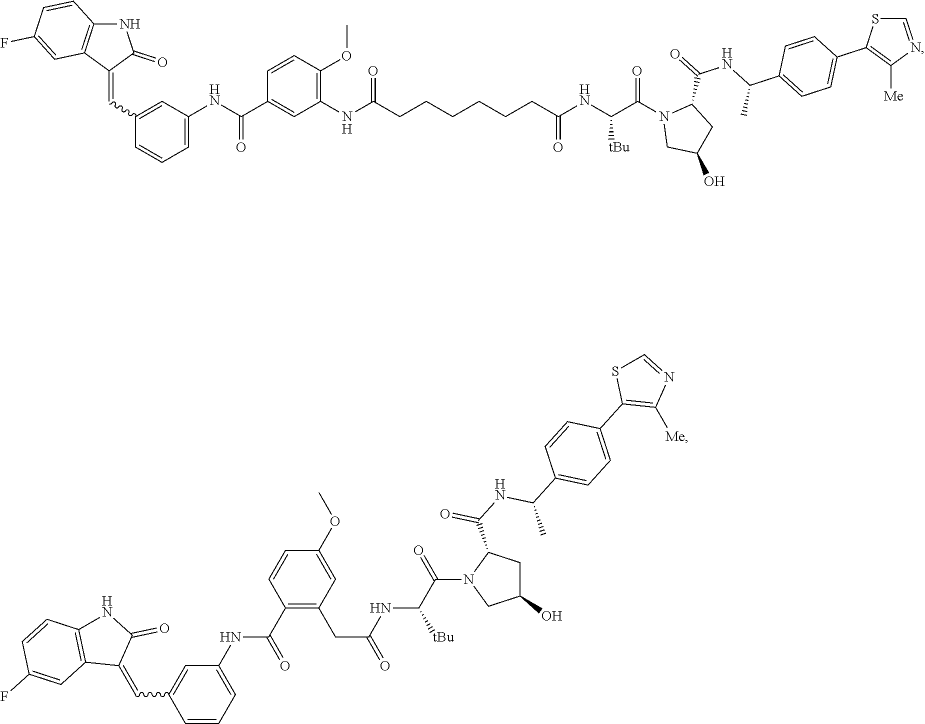 [SMILES: COc1ccc(C(=O)Nc2cccc(C=C3C(=O)Nc4ccc(F)cc43)c2)cc1NC(=O)CCCCCCC(=O)N[C@H](C(=O)N1C[C@H](O)C[C@H]1C(=O)N[C@@H](C)c1ccc(-c2scnc2C)cc1)C(C)(C)C ; COc1ccc(C(=O)Nc2cccc(C=C3C(=O)Nc4ccc(F)cc43)c2)c(CC(=O)N[C@H](C(=O)N2C[C@H](O)C[C@H]2C(=O)N[C@@H](C)c2ccc(-c3scnc3C)cc2)C(C)(C)C)c1]

or any combination thereof.

### General Synthetic Method

### Scaffolds

In one aspect, a scaffold can be synthesized using the following general schemes, wherein substituents on rings A, B, and C can be modified by methods known in the art and/or by using differentially-substituted starting materials.

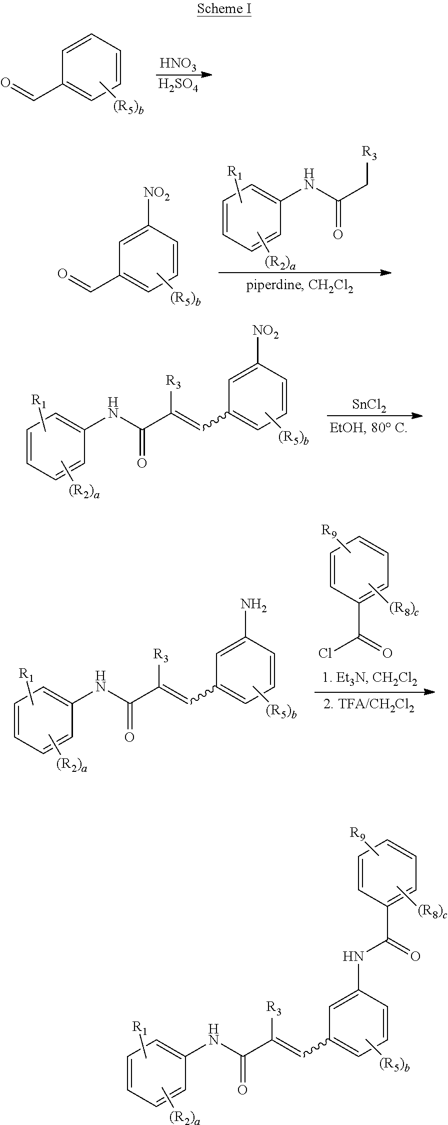

wherein variables have the identities disclosed herein.

In another aspect, although 6-membered aryl rings are shown in Scheme 1, aryl rings and/or other substituents can vary as described herein without significant departures from the general method. Solvents, temperatures, presence or absence of protecting groups, and other reaction conditions may vary according to the specific substituents in the compound being synthesized. Exemplary methods for producing specific scaffolds, as well as characterization information, are provided in the Examples.

### PROTACs

In one aspect, when R₉ is Formula II, a scaffold as disclosed herein can first be synthesized including an azide (—N₃) group at position R₉ and the following general scheme can be followed to generate a PROTAC:

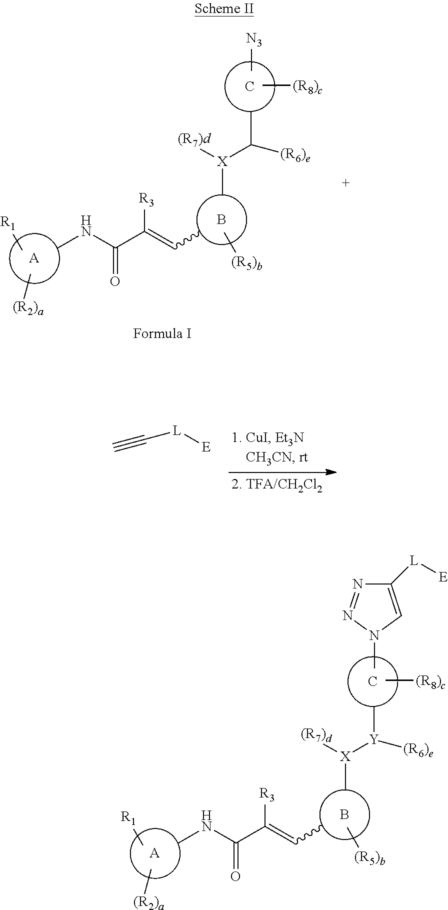

where Formula I, L, E, and other variables are as disclosed previously herein.

Exemplary methods for producing specific PROTACs, as well as characterization information, are provided in the Examples. In some aspects, PROTACs incorporating amide or sulfonamide groups in the linking moieties are synthesized by analogous methods for forming amide or sulfonamide bonds known in the art. Solvents, temperatures, presence or absence of protecting groups, and other reaction conditions may vary according to the specific substituents in the compound being synthesized.

### Therapeutic Agents

As referred to herein, “ADRM1” is a gene encoding proteasomal ubiquitin receptor ADRM1/Rpn13. ADRM1 encodes subunit Rpn13 (also referred to herein as RPN13) of the base sub-complex of the 19S regulatory particle of the 26S proteasome complex. “Rpn13” functions as a ubiquitin receptor; “hRpn13” refers specifically to the version of this protein in humans but “Rpn13” is used interchangeably with “hRpn13”. RPN13 “variant,” “mutated,” or “mutant” refers to ADRM1 gene products in which the amino acid sequence of the protein RPN13 product is altered, as typically occurs in cancer. In one aspect, targeting hRpn13 is a promising strategy in cancer research. “Rpn2,” (also called PSMD1, non-ATPase 1, or S1) meanwhile is a large protein with a 14 amino acid extension that binds to Rpn13 causing it to be a part of the 26S proteasome complex. Rpn2 is part of the base sub-complex of the 19S regulatory particle that includes Rpn13 (“hRpn2” again refers to the version of this protein in humans).

“N-terminal Pru” (where “Pru” is short for “Pleckstrin-like receptor for ubiquitin”) as used herein refers to an N-terminal region of hRpn13 that binds to hRpn2 and also dynamically to ubiquitin chains. “Rpn13-Pru,” meanwhile, refers to a truncated version of Rpn13 having a Pru motif, but missing the C-terminal domain in Rpn13 that binds to deubiquitinating enzyme UCHL5, also called Uch37. “Rpn13-Pru” is an example of a variant Rpn13 protein product, but also of a naturally occurring event in which the proteasome has cleaved the full length Rpn13 protein to generate a truncated Rpn13 protein. In some aspects, Rpn13-Pru can be a biomarker for cancers. In one aspect, Rpn13-Pru can be a biomarker for dysregulated proteasome activity. In one aspect, hRpn13 and/or hRpn13-Pru refer to the human versions of these gene products, while Rpn13 and Rpn13-Pru refer to the gene products more generally. In a further aspect, when Rpn13 and/or Rpn13-Pru are referred to in the context of a human subject, it can be assumed that these terms are being used interchangeably with hRpn13 and hRpn13-Pru. In one aspect, variants of Rpn13 and/or Rpn13-Pru, including mutants and variants containing the N-terminal Pru domain and/or missing the C-terminal domain in Rpn13 that binds to deubiquitinating enzyme UCHL5, are also associated with cancers and can be used as biomarkers for the same. Further in this aspect, the disclosed molecules and PROTACs exhibit binding affinity to and can be used to target these variants as well.

“E3 ubiquitin ligase” is a protein that recruits an E2 ubiquitin-conjugating enzyme that is pre-loaded with ubiquitin and that catalyzes the transfer of ubiquitin to the protein to be degraded. In one aspect, disclosed herein are PROTACs that include an E3 ubiquitin ligase-binding ligand linked to a scaffold configured to bind to hRpn13, thereby causing the ubiquitination and/or degradation of hRpn13 and/or hRpn13-Pru.

As used herein, a “PROTAC” is a proteolysis targeting chimera, or a small molecule having two active domains and a linker, wherein the PROTAC is capable of causing the ubiquitination and/or degradation or inactivation of unwanted proteins. In a further aspect, as a mechanism of action, a PROTAC activates intracellular proteolysis. In one aspect, one of the active domains engages an E3 ubiquitin ligase and the other binds the target protein (e.g., hRpn13). Disclosed herein are PROTACs useful in recruiting VHL and other tumor-suppressor proteins to assist in the degradation of hRpn13. Also disclosed herein are scaffold molecules useful as the target-protein binding domain in PROTACs. In some aspects, the scaffold molecules also have anti hRpn13 activity.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, “attached” can refer to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, π-π interactions, cation-π interactions, anion-π interactions, polar π-interactions, and hydrophobic effects.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a hematological malignancy, breast cancer, and/or another solid malignancy. The effect can be therapeutic in terms of a partial or complete cure of a disease, condition, symptom or adverse effect attributed to the disease, disorder, or condition. The term “treatment” as used herein can include any treatment of a hematological malignancy, breast cancer, and/or another solid tumor in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1-to-C 6 alkyl esters and C 5-to-C 7 cycloalkyl esters, although C 1-to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1-to-C 6 alkyl amines and secondary C 1-to-C 6 dialkyl amines. In the case of secondary amines, the amine can also be in the form of a 5- or 6-membered heterocycle containing one nitrogen atom. Amides derived from ammonia, C 1-to-C 3 alkyl primary amides and C 1-to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

Described herein are hRpn13 binders and/or PROTACs that have therapeutic or clinical utility. Also described herein are methods of synthesizing the hRpn13 binders and PROTACs. Also described herein are methods of administering the hRpn13 binders and PROTACs to a subject in need thereof. In some aspects, the subject can have cancer. In other aspects, the subject has dysregulated proteasome activity, which links either to cancer or other diseases. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Compounds

In various aspects, it is contemplated herein that the disclosed compounds further comprise their bioisosteric equivalents. The term “bioisosteric equivalent” refers to compounds or groups that possess near equal molecular shapes and volumes, approximately the same distribution of electrons, and which exhibit similar physical and biological properties. Examples of such equivalents are: (i) fluorine vs. hydrogen, (ii) oxo vs. thia, (iii) hydroxyl vs. amide, (iv) carbonyl vs. oxime, (v) carboxylate vs. tetrazole. Examples of such bioisosteric replacements can be found in the literature and examples of such are: (i) Burger A, Relation of chemical structure and biological activity; in Medicinal Chemistry Third ed., Burger A, ed.; Wiley-Interscience; New York, 1970, 64-80; (ii) Burger, A.; “Isosterism and bioisosterism in drug design”; Prog. Drug Res. 1991, 37, 287-371; (iii) Burger A, “Isosterism and bioanalogy in drug design”, Med, Chem Res. 1994, 4, 89-92; (iv) Clark R D, Ferguson A M, Cramer R D, “Bioisosterism and molecular diversity”, Perspect. Drug Discovery Des. 1998, 9/10/11, 213-224; (v) Koyanagi T, Haga T, “Bioisosterism in agrochemicals”, ACS Symp. Ser. 1995, 584, 15-24; (vi) Kubinyi H, “Molecular similarities. Part 1. Chemical structure and biological activity”, Pharm. UnsererZeit 1998, 27, 92-106; (vii) Lipinski C A.; “Bioisosterism in drug design”; Annu. Rep. Med. Chem. 1986, 21, 283-91; (viii) Patani G A, LaVoie E J, “Bioisosterism: A rational approach in drug design”, Chem. Rev. (Washington, D.C.) 1996, 96, 3147-3176; (ix) Soskic V, Joksimovic J, “Bioisosteric approach in the design of new dopaminergic/serotonergic ligands”, Curr. Med. Chem, 1998, 5, 493-512 (x) Thornber C W, “Isosterism and molecular modification in drug design”, Chem. Soc. Rev. 1979, 8, 563-80.

In further aspects, bioisosteres are atoms, ions, or molecules in which the peripheral layers of electrons can be considered substantially identical. The term bioisostere is usually used to mean a portion of an overall molecule, as opposed to the entire molecule itself. Bioisosteric replacement involves using one bioisostere to replace another with the expectation of maintaining or slightly modifying the biological activity of the first bioisostere. The bioisosteres in this case are thus atoms or groups of atoms having similar size, shape and electron density. Preferred bioisosteres of esters, amides or carboxylic acids are compounds containing two sites for hydrogen bond acceptance. In one embodiment, the ester, amide or carboxylic acid bioisostere is a 5-membered monocyclic heteroaryl ring, such as an optionally substituted 1H-imidazolyl, an optionally substituted oxazolyl, 1H-tetrazolyl, [1,2,4]triazolyl, or an optionally substituted [1,2,4]oxadiazolyl.

In various aspects, the disclosed compounds can possess at least one center of asymmetry, they can be present in the form of their racemates, in the form of the pure enantiomers and/or diastereomers or in the form of mixtures of these enantiomers and/or diastereomers. The stereoisomers can be present in the mixtures in any arbitrary proportions. In some aspects, provided this is possible, the disclosed compounds can be present in the form of the tautomers.

Thus, methods which are known per se can be used, for example, to separate the disclosed compounds which possess one or more chiral centers and occur as racemates into their optical isomers, i.e., enantiomers or diastereomers. The separation can be effected by means of column separation on chiral phases or by means of recrystallization from an optically active solvent or using an optically active acid or base or by means of derivatizing with an optically active reagent, such as an optically active alcohol, and subsequently cleaving off the residue.

In various aspects, the disclosed compounds can be in the form of a co-crystal. The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Preferred co-crystals include p-toluenesulfonic acid and benzenesulfonic acid.

The term “pharmaceutically acceptable co-crystal” means one that is compatible with the other ingredients of the formulation and not deleterious to the recipient thereof.

In a further aspect, the disclosed compounds can be isolated as solvates and, in particular, as hydrates of a disclosed compound, which can be obtained, for example, by crystallization from a solvent or from aqueous solution. In this connection, one, two, three or any arbitrary number of solvate or water molecules can combine with the compounds according to the invention to form solvates and hydrates.

The disclosed compounds can be used in the form of salts derived from inorganic or organic acids. Pharmaceutically acceptable salts include salts of acidic or basic groups present in the disclosed compounds. Suitable pharmaceutically acceptable salts include base addition salts, including alkali metal salts, e.g., sodium or potassium salts; alkaline earth metal salts, e.g., calcium or magnesium salts; and salts formed with suitable organic ligands, e.g., quaternary ammonium salts, which may be similarly prepared by reacting the drug compound with a suitable pharmaceutically acceptable base. The salts can be prepared in situ during the final isolation and purification of the compounds of the present disclosure; or following final isolation by reacting a free base function, such as a secondary or tertiary amine, of a disclosed compound with a suitable inorganic or organic acid; or reacting a free acid function, such as a carboxylic acid, of a disclosed compound with a suitable inorganic or organic base.

Acidic addition salts can be prepared in situ during the final isolation and purification of a disclosed compound, or separately by reacting moieties comprising one or more nitrogen groups with a suitable acid. In various aspects, acids which may be employed to form pharmaceutically acceptable acid addition salts include such inorganic acids as hydrochloric acid, sulfuric acid and phosphoric acid and such organic acids as oxalic acid, maleic acid, succinic acid and citric acid. In a further aspect, salts further include, but are not limited, to the following: hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, butyrate, camphorate, camphorsulfonate, digluconate, glycerophosphate, hemisulfate, heptanoate, hexanoate, fumarate, hydrochloride, 2-hydroxyethanesulfonate (isethionate), nicotinate, 2-naphthalenesulfonate, oxalate, pectinate, persulfate, 3-phenylpropionate, picrate, pivalate, propionate, succinate, tartrate, thiocyanate, phosphate, glutamate, bicarbonate, undecanoate, and pamoate (i.e., 1,1′-methylene-bis-(2-hydroxy-3-naphthoate)) salts. Also, basic nitrogen-containing groups can be quaternized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others.

Basic addition salts can be prepared in situ during the final isolation and purification of a disclosed compound, or separately by reacting carboxylic acid moieties with a suitable base such as the hydroxide, carbonate or bicarbonate of a pharmaceutical acceptable metal cation or with ammonia, or an organic primary, secondary or tertiary amine. Pharmaceutical acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like. In further aspects, bases which may be used in the preparation of pharmaceutically acceptable salts include the following: ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

### Pharmaceutical Compositions

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitoneally, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In various aspects, a disclosed compound comprising an acidic group or moiety, e.g., a carboxylic acid group, can be used to prepare a pharmaceutically acceptable salt. For example, such a disclosed compound may comprise an isolation step comprising treatment with a suitable inorganic or organic base. In some cases, it may be desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base compounds are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, a disclosed compound comprising a protonatable group or moiety, e.g., an amino group, can be used to prepare a pharmaceutically acceptable salt. For example, such a disclosed compound may comprise an isolation step comprising treatment with a suitable inorganic or organic acid. In some cases, it may be desirable in practice to initially isolate a compound from the reaction mixture as a pharmaceutically unacceptable salt and then simply convert the latter back to the free base compound by treatment with a basic reagent, and subsequently convert the free base to a pharmaceutically acceptable acid addition salt. These acid addition salts can be readily prepared using conventional techniques, e.g., by treating the corresponding basic compounds with an aqueous solution containing the desired pharmacologically acceptable anions and then evaporating the resulting solution to dryness, preferably under reduced pressure. Alternatively, they also can be prepared by treating the free base form of the disclosed compound with a suitable pharmaceutically acceptable non-toxic inorganic or organic acid.

Acids that can be used to prepare the pharmaceutically acceptable acid-addition salts of the base compounds are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt comprises an anion formed from hydrobromic, hydrochloric, maleic, phosphoric, sulfuric, and tartaric acids.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe fOr Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In the treatment conditions which require inhibition or degradation of hRpn13 activity an appropriate dosage level will generally be about 0.01 to 1000 mg per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The present disclosure is further directed to a method for the manufacture of a medicament for modulating hRpn13 activity (e.g., treatment of one or more cancers or other disorders associated with hRpn13 dysfunction) in subjects (e.g., humans), wherein the method includes the steps of combining one or more disclosed compounds, products, or compositions with a pharmaceutically acceptable carrier or diluent.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

As already mentioned, the present disclosure relates to a pharmaceutical composition comprising a therapeutically effective amount of a disclosed compound, a product of a disclosed method of making, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

As already mentioned, the present disclosure also relates to a pharmaceutical composition comprising a disclosed compound, a product of a disclosed method of making, a pharmaceutically acceptable salt, a hydrate thereof, a solvate thereof, a polymorph thereof, and one or more other drugs in the treatment, prevention, control, amelioration, or reduction of risk of diseases or conditions for a disclosed compound or the other drugs may have utility as well as to the use of such a composition for the manufacture of a medicament. The present disclosure also relates to a combination of disclosed compound, a product of a disclosed method of making, a pharmaceutically acceptable salt, a hydrate thereof, a solvate thereof, a polymorph thereof, and an hRpn13 binder or PROTAC. The present disclosure also relates to such a combination for use as a medicine. The present disclosure also relates to a product comprising (a) disclosed compound, a product of a disclosed method of making, a pharmaceutically acceptable salt, a hydrate thereof, a solvate thereof, a polymorph thereof, and (b) an additional chemotherapeutic agent, as a combined preparation for simultaneous, separate or sequential use in the treatment or prevention of a condition in a mammal, including a human, the treatment or prevention of which is affected or facilitated by the modulatory effect of the disclosed compound and the additional therapeutic agent. The different drugs of such a combination or product may be combined in a single preparation together with pharmaceutically acceptable carriers or diluents, or they may each be present in a separate preparation together with pharmaceutically acceptable carriers or diluents.

In a further aspect, the present disclosure provides methods of treatment comprising administration of a therapeutically effective amount of a disclosed compound or pharmaceutical composition as disclosed herein above to a subject in need thereof.

In one aspect, disclosed herein is a pharmaceutical composition including a therapeutically effective amount of a compound disclosed herein or a pharmaceutically acceptable salt, solvate, or polymorph thereof, and a pharmaceutically acceptable carrier.

### Methods for Detection of Cancers in Subjects

In one aspect, disclosed herein are methods for detecting cancers associated with Rpn13 or Rpn13-Pru in a subject, the method including administering a fluorescently labeled disclosed compound to the subject, wherein the fluorescently labeled compound localizes with the cancer and visualizing and/or quantifying fluorescence in the sample collected from the subject.

In another aspect, provided herein is a method for detecting a cancer and/or proteasome dysfunction in a subject, the method including measuring an Rpn13-Pru biomarker in a sample from the subject to determine the presence, absence, or level of the biomarker, and correlating the measurement of the presence, absence, or level of the biomarker to the cancer. In some aspects, the sample can be blood, serum, plasma, or a solid tissue sample. In any of these aspects, the biomarker can be measured using mass spectrometry. In one aspect, the cancer can be selected from multiple myeloma, lymphoma, mantle cell lymphoma, acute leukemia, cancers associated with human papillomavirus, colorectal cancer, gastric cancer, ovarian cancer, liver cancer, breast cancer, cervical cancer, prostate cancer, and pancreatic cancer, or any combination thereof.

### Methods for Treatment of Cancers in Subjects

In one aspect, disclosed herein is a method for the treatment of a cancer in a subject, the method including the step of administering to the subject a therapeutically effective amount of at least one disclosed compound, or a pharmaceutically acceptable salt thereof, or the disclosed pharmaceutical composition. In some aspects, the subject is a human. In another aspect, the subject has been diagnosed with a need for treatment of the cancer prior to the administering step. In some aspects, the method further includes the step of identifying a subject in need of treatment of the cancer. In one aspect, the cancer is selected from multiple myeloma, lymphoma, mantle cell lymphoma, acute leukemia, cancers associated with human papillomavirus, colorectal cancer, gastric cancer, ovarian cancer, liver cancer, breast cancer, cervical cancer, pancreatic cancer, prostate cancer, or any combination thereof.

In another aspect, disclosed herein is a method for inhibiting the activity of Rpn13 or Rpn13-Pru in a subject, including the step of administering to the subject a therapeutically effective amount of at least one disclosed compound, or a pharmaceutically acceptable salt thereof, or a disclosed pharmaceutical composition. In one aspect, the subject is a human.

In a further aspect, the method further includes the step of administering to the subject one or more additional agents known to decrease the activity of Rpn13 or Rpn13-Pru. In yet another aspect, the method includes the step of administering one or more additional anti-cancer agents to the subject. In one aspect, the anti-cancer agent can be or include carfilzomib, bortezomib, ixazomib, disulfiram, marizomib, oprozomib, epoxomicin, MG132, KZR—616, KZR—504, PKS2279, PKS2252, another proteasome or immunoproteasome inhibitor, or any combination thereof.

### Kits

In a further aspect, the present disclosure relates to kits comprising at least one disclosed compound, or a pharmaceutically acceptable salt, hydrate, solvate, or polymorph thereof, and one or more of: (a) at least one agent known to decrease Rpn13 or Rpn13-Pru activity; (b) at least one agent known to treat a cancer associated with aberrant Rpn13 or Rpn13-Pru activity and/or assess the presence of hRpn13-Pru; (c) instructions for treating a cancer associated with aberrant hRpn13 activity and/or the presence of hRpn13-Pru; or (d) instructions for administering the compound in connection with another cancer therapy.

The disclosed compounds and/or pharmaceutical compositions comprising the disclosed compounds can conveniently be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient. In further aspects, a kit can include optional components that aid in the administration of the unit dose to patients, such as vials for reconstituting powder forms, syringes for injection, customized IV delivery systems, inhalers, etc. Additionally, a kit can contain instructions for preparation and administration of the compositions. The kit can be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

In a further aspect, the disclosed kits can be packaged in a daily dosing regimen (e.g., packaged on cards, packaged with dosing cards, packaged on blisters or blow-molded plastics, etc.). Such packaging promotes products and increases patient compliance with drug regimens.

Such packaging can also reduce patient confusion. The present invention also features such kits further containing instructions for use.

In a further aspect, the present disclosure also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

In various aspects, the disclosed kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using or treating, and/or the disclosed compositions.

In one aspect, disclosed herein is a kit containing at least one disclosed compound or a pharmaceutically acceptable salt thereof and one or more of (a) at least one agent known to decrease the activity of Rpn13 or Rpn13-Pru, and (b) at least one agent known to treat multiple myeloma, lymphoma, mantle cell lymphoma, acute leukemia, cancers associated with human papillomavirus, colorectal cancer, gastric cancer, ovarian cancer, liver cancer, breast cancer, cervical cancer, pancreatic cancer, prostate cancer, or a combination thereof.

In one aspect, the disclosed compound and the at least one agent are co-formulated and/or co-packaged.

In one aspect, the at least one agent can be carfilzomib, bortezomib, ixazomib, disulfiram, marizomib, oprozomib, epoxomicin, MG132, KZR—616, KZR—504, PKS2279, PKS2252, another proteasome or immunoproteasome inhibitor, or any combination thereof.

### Research Tools

The disclosed compounds and pharmaceutical compositions have activity as inhibitors of hRpn13 and/or as compounds that target hRpn13 by binding and, subsequently, by recruiting ubiquitinating enzymes and/or proteolytic enzymes to ubiquitinate and/or degrade hRpn13. As such, the disclosed compounds are also useful as research tools. Accordingly, one aspect of the present disclosure relates to a method of using a compound of the invention as a research tool, the method comprising conducting a biological assay using a compound of the invention. Compounds of the invention can also be used to evaluate new chemical compounds. Thus another aspect of the invention relates to a method of evaluating a test compound in a biological assay, comprising: (a) conducting a biological assay with a test compound to provide a first assay value; (b) conducting the biological assay with a compound of the invention to provide a second assay value; wherein step (a) is conducted either before, after or concurrently with step (b); and (c) comparing the first assay value from step (a) with the second assay value from step (b). Exemplary biological assays include an IC₅₀ assay that can be conducted in vitro or in a cell culture system. Still another aspect of the invention relates to a method of studying a biological system, e.g., a model animal for a clinical condition, or biological sample comprising an hRpn13 protein, the method comprising: (a) contacting the biological system or sample with a compound of the invention; and (b) determining the effects caused by the compound on the biological system or sample.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

The present disclosure can be described in accordance with the following numbered aspects, which should not be confused with the claims.

Aspect 1. A compound comprising a structure of Formula I:

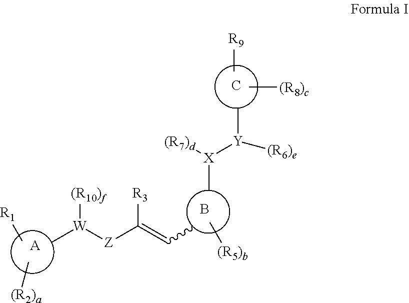

wherein A, B, and C independently comprise an aryl or heteroaryl ring having 5-10 members;
wherein X and Y independently comprise carbon, oxygen, nitrogen, sulfur, a carbonyl group, or a sulfonyl group;
wherein each instance of R₆ and R₇ is absent or independently comprises hydrogen, halogen, hydroxyl, trifluoromethyl, C1-C6 alkylamino, C1-C6 alkoxy, substituted or unsubstituted C1-C6 alkyl, a substituted or unsubstituted phenyl group, or Formula II;

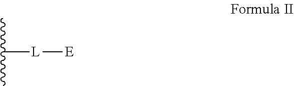

wherein L comprises a linker moiety;
wherein E comprises an E3 ubiquitin ligase targeting moiety, a bridging molecule to a ubiquitin E3 ligase complex, an E2 ubiquitin conjugating enzyme targeting molecule, an autophagy-targeting chimera, or a proteasome subunit targeting molecule;
wherein when at least one R₇ is present, d is 1 or 2; and wherein when at least one R₆ is present, e is 1 or 2;
wherein R₁ comprises —SO₂NH₂, a carboxylic acid group, fluorine, a trifluoromethyl group, or a tetrazole;
wherein each instance of R₂ independently comprises hydrogen, halogen, hydroxyl, trifluoromethyl, C1-C6 alkylamino, C1-C6 alkoxy, or substituted or unsubstituted C1-C6 alkyl, and a is from 1 to 4;
wherein R₃ comprises a cyano group, —S(═O)₂—R₄, —C(═O)—R₄, —C(═O)—OR₄, —C(═O)—N—R₄,R₄′, or —S(═O)₂—NH₂;
wherein R₄ and R₄′ comprise hydrogen, halogen, hydroxyl, trifluoromethyl, C1-C6 alkylamino, C1-C6 alkoxy, substituted or unsubstituted C1-C6 alkyl, or a substituted or unsubstituted phenyl group;
wherein each instance of R₅ independently comprises hydrogen, halogen, hydroxyl, trifluoromethyl, C1-C6 alkylamino, C1-C6 alkoxy, substituted or unsubstituted C1-C6 alkyl, a substituted or unsubstituted phenyl group, or Formula II, and wherein b is from 1 to 5;
wherein each instance of R₆ independently comprises hydrogen, halogen, hydroxyl, trifluoromethyl, C1-C6 alkylamino, C1-C6 alkoxy, or substituted or unsubstituted C1-C6 alkyl; and wherein c is from 1 to 5; and
wherein R₉ comprises hydrogen, halogen, hydroxyl, trifluoromethyl, substituted or unsubstituted C1-C6 alkylamino, substituted or unsubstituted C1-C6 alkyl, an azide group, or Formula II;
wherein Z comprises a carbonyl group or a sulfonyl group;
wherein W comprises carbon, oxygen, nitrogen, or sulfur;
wherein each instance of R₁₀ is absent or independently comprises hydrogen, halogen, hydroxyl, trifluoromethyl, C1-C6 alkylamino, a substituted or unsubstituted phenyl group, or Formula II; and wherein f is from 0 to 2; and
wherein the compound is not XL5 and is not XL23.

Aspect 2. The compound of aspect 1, wherein the compound comprises Formula Ia, Formula Ib, or any combination thereof:

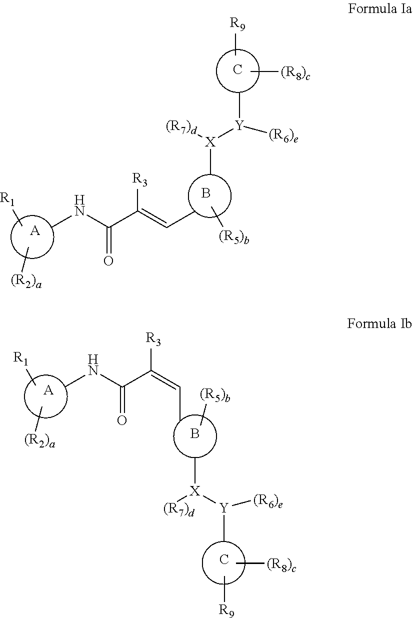

Aspect 3. The compound of aspect 1 or 2, wherein A is a substituted or unsubstituted phenyl group.

Aspect 4. The compound of any one of aspects 1-3, wherein B is a substituted or unsubstituted phenyl group.

Aspect 5. The compound of any one of aspects 1-4, wherein C is a substituted or unsubstituted phenyl or pyridyl group.

Aspect 6. The compound of any one of aspects 1-5, wherein X is nitrogen.

Aspect 7. The compound of any one of aspects 1-6, wherein R₇ is hydrogen and d is 1.

Aspect 8. The compound of any one of aspects 1-7, wherein Y is a carbonyl group and R₆ is absent.

Aspect 9. The compound of any one of aspects 1-8, wherein R₁ is —SO₂NH₂ or a carboxylic acid group.

Aspect 10. The compound of any one of aspects 1-9, wherein each R₂ is independently hydrogen, trifluoromethyl, methylamino, or methoxy, and wherein a is 4.

Aspect 11. The compound of any one of aspects 1-10, wherein R₃ is cyano.

Aspect 12. The compound of any one of aspects 1-11, wherein each R₅ is independently hydrogen, trifluoromethyl, or methylamino and wherein b is 4.

Aspect 13. The compound of any one of aspects 1-12, wherein each R₆ is independently chloro, hydrogen, or hydroxyl, and wherein c is 4.

Aspect 14. The compound of any one of aspects 1-13, wherein R₉ is hydrogen, methyl, methylamino, trifluoromethyl, —NHCH₂COOH, or Formula II.

Aspect 15. The compound of any one of aspects 1-14, wherein at least one of R₅, R₆, R₇, or R₉ comprises Formula II and wherein L comprises:

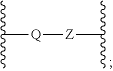

wherein Q comprises a triazole, an amide, a C1-C4 alkyl amide, a sulfonamide, or substituted or unsubstituted spirocyclic rings;
and wherein Z comprises an alkyl group, an alkylene group, a polyether group, or any combination thereof.

Aspect 16. The compound of aspect 15, wherein Z comprises:

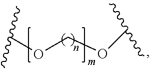

wherein n is 2 or 3 and wherein m is from 1 to 10; or

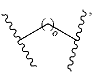

wherein o is from 0 to 10.

Aspect 17. The compound of any one of aspects 1-14, wherein R₉ is Formula II and wherein L is:

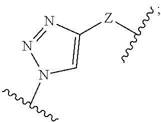

and wherein Z is an alkyl group, an alkylene group, a polyether group, or any combination thereof.

Aspect 18. The compound of any one of aspects 1-14, wherein R₉ is Formula II and wherein L is:

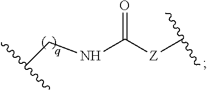

wherein q is 0 or 1;
and wherein Z is an alkyl group, an alkylene group, a polyether group, or any combination thereof.

Aspect 19. The compound of any one of aspects 1-14, wherein R₉ is Formula II and wherein L is:

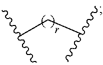 [SMILES: *C*]

and wherein r is from 1 to 5.

Aspect 20. The compound of any one of aspects 15-19, wherein Q includes substituted or unsubstituted spirocyclic rings selected from:

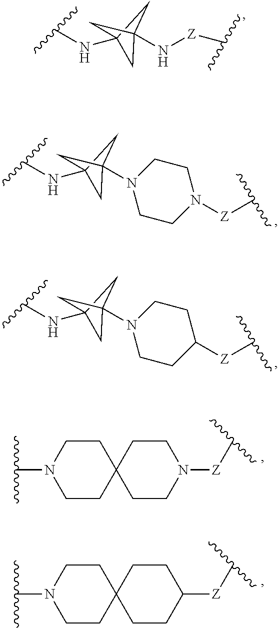

or any combination thereof.

Aspect 21. The compound of any one of aspects 15-19, wherein L is

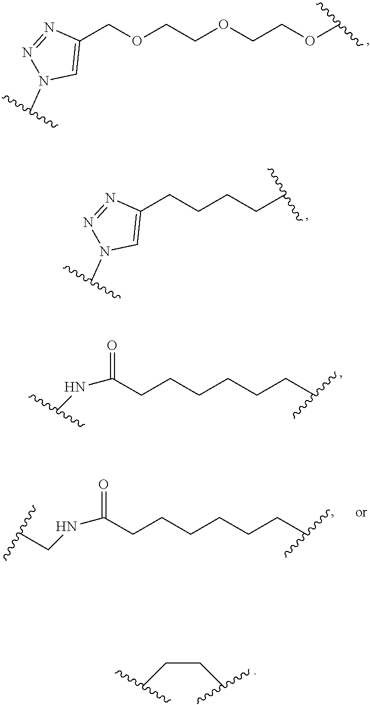

Aspect 22. The compound of any one of aspects 15-19 or 21, wherein the compound is represented by a structure of Formula III:

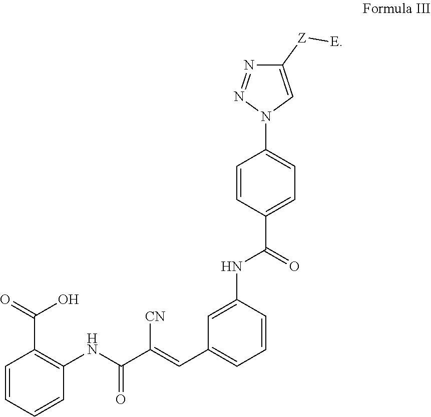

Aspect 23. The compound of any one of aspects 1-22, wherein R₉ is Formula II and wherein E comprises a cereblon-targeting molecule, a von Hippel-Lindau targeting molecule, an IAP E3 ligase targeting molecule, an MDM2-targeting E3 ligase, an autophagy targeting chimera (AUTAC), or an Rpn11-targeting molecule.

Aspect 24. The compound of aspect 23, wherein the cereblon-targeting molecule is thalidomide, lenalidomide, pomalidomide, iberdomide, or apremilast.

Aspect 25. The compound of aspect 23, wherein the AUTAC is:

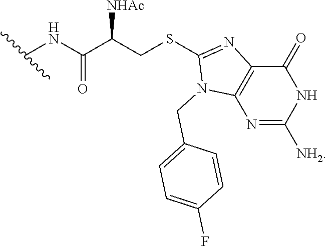 [SMILES: *NC(=O)[C@H](CSc1nc2c(=O)[nH]c(N)nc2n1Cc1ccc(F)cc1)NC(C)=O]

Aspect 26. The compound of aspect 23, wherein the Rpn11-targeting molecule is capzimin or a derivative thereof.

Aspect 27. The compound of any one of aspects 1-23, wherein R₉ is Formula II and wherein E is

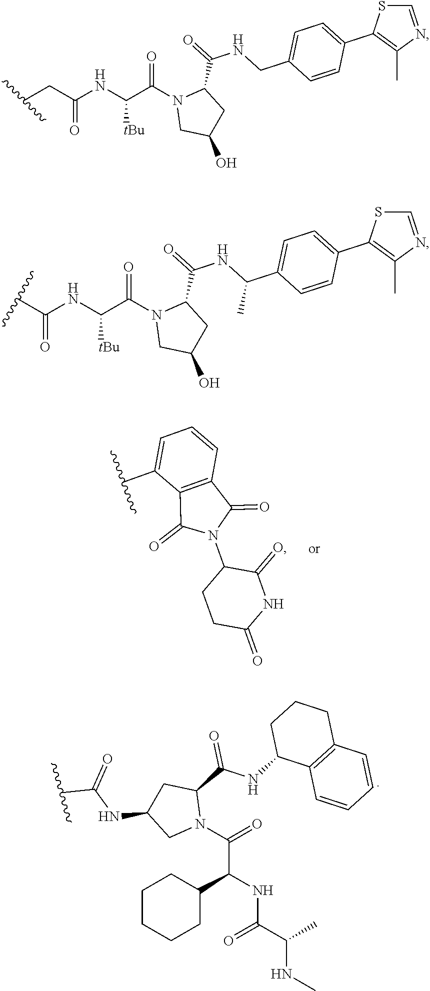 [SMILES: *CC(=O)N[C@H](C(=O)N1C[C@H](O)C[C@H]1C(=O)NCc1ccc(-c2scnc2C)cc1)C(C)(C)C ; *C(=O)N[C@H](C(=O)N1C[C@H](O)C[C@H]1C(=O)N[C@@H](C)c1ccc(-c2scnc2C)cc1)C(C)(C)C ; *c1cccc2c1C(=O)N(C1CCC(=O)NC1=O)C2=O ; *C(=O)N[C@H]1C[C@@H](C(=O)N[C@@H]2CCCc3ccccc32)N(C(=O)[C@@H](NC(=O)[C@H](C)NC)C2CCCCC2)C1]

Aspect 28. The compound of any one of aspects 1-23 or 27 having a structure represented by a formula:

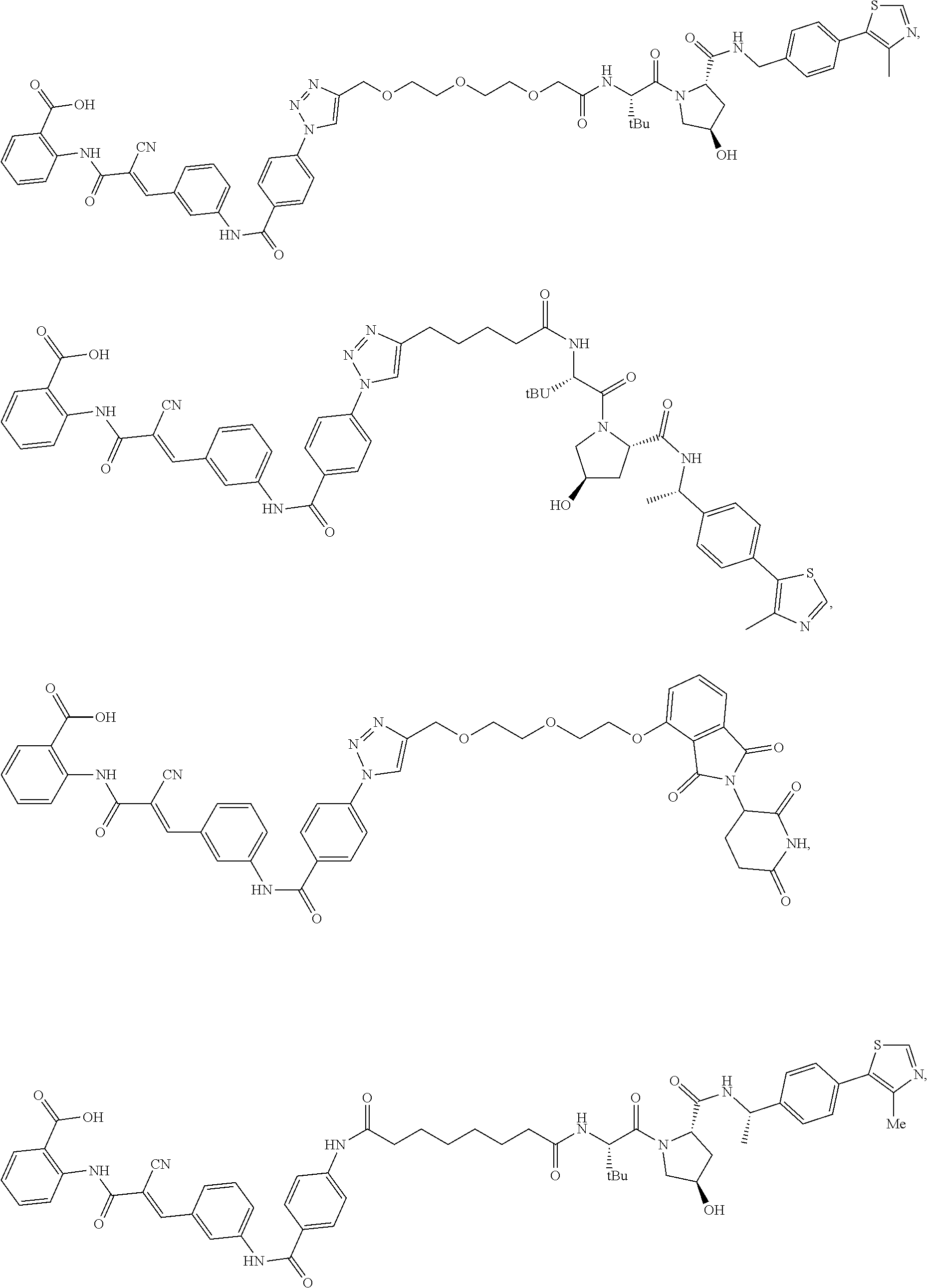

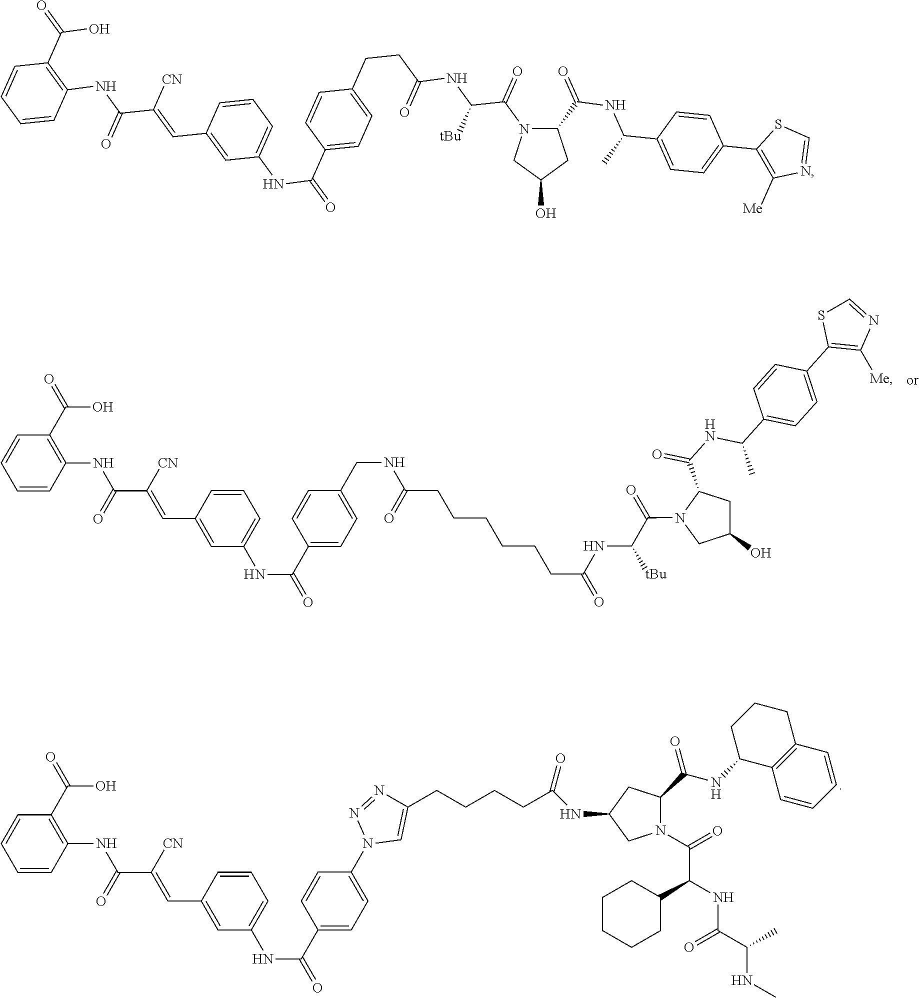 [SMILES: Cc1ncsc1-c1ccc([C@H](C)NC(=O)[C@@H]2C[C@@H](O)CN2C(=O)[C@@H](NC(=O)CCc2ccc(C(=O)Nc3cccc(/C=C(\C#N)C(=O)Nc4ccccc4C(=O)O)c3)cc2)C(C)(C)C)cc1 ; Cc1ncsc1-c1ccc([C@H](C)NC(=O)[C@@H]2C[C@@H](O)CN2C(=O)[C@@H](NC(=O)CCCCCCC(=O)NCc2ccc(C(=O)Nc3cccc(/C=C(\C#N)C(=O)Nc4ccccc4C(=O)O)c3)cc2)C(C)(C)C)cc1 ; CN[C@@H](C)C(=O)N[C@H](C(=O)N1C[C@@H](NC(=O)CCCCc2cn(-c3ccc(C(=O)Nc4cccc(/C=C(\C#N)C(=O)Nc5ccccc5C(=O)O)c4)cc3)nn2)C[C@H]1C(=O)N[C@@H]1CCCc2ccccc21)C1CCCCC1]

Aspect 29. The compound of any one of aspects 1-14, having a structure represented by a formula:

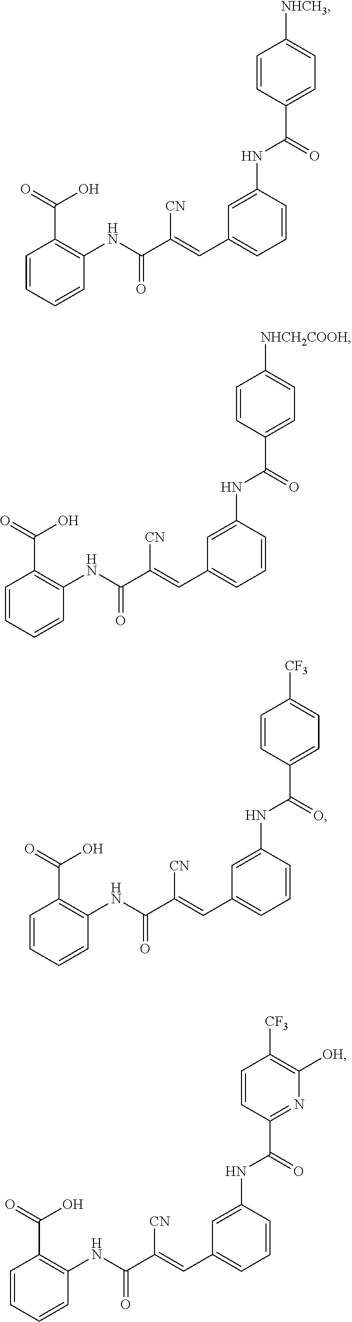

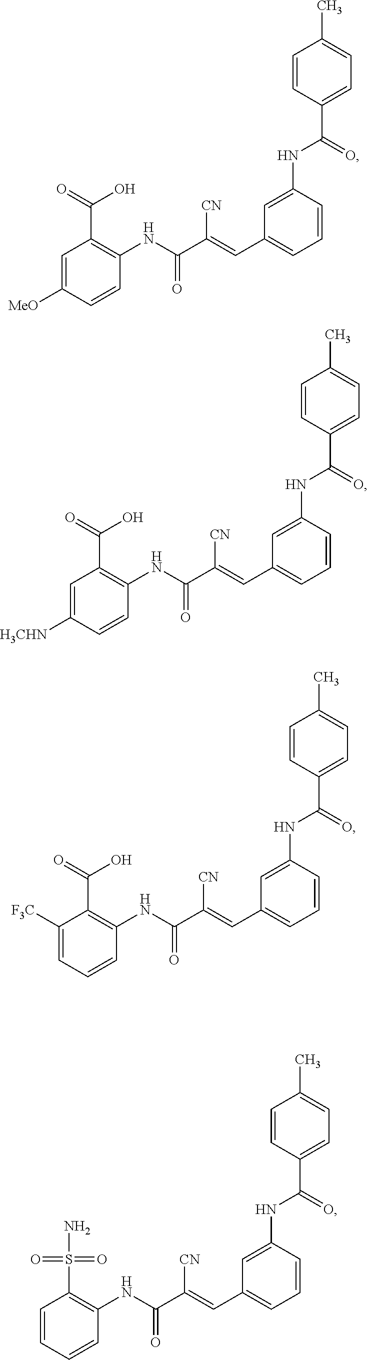

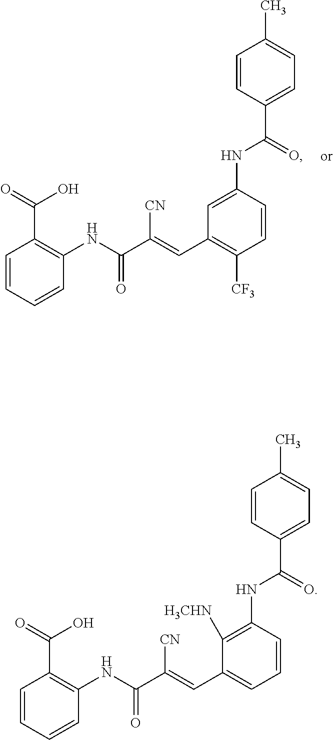

Aspect 30. A compound comprising a structure of Formula IV:

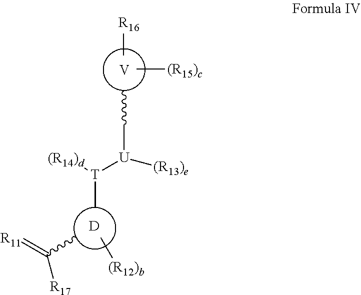

wherein D and V independently comprise an aryl or heteroaryl ring having 5-10 members;
wherein T and U independently are carbon, oxygen, nitrogen, sulfur, a carbonyl group, or a sulfonyl group;
wherein each instance of R₁₃ and R₁₄ is absent or independently is hydrogen, halogen, hydroxyl, trifluoromethyl, C1-C6 alkylamino, C1-C6 alkoxy, substituted or unsubstituted C1-C6 alkyl, a substituted or unsubstituted phenyl group, or Formula II;

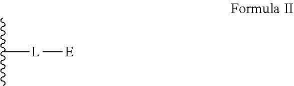

wherein L comprises a linker moiety;
wherein E comprises an E3 ubiquitin ligase targeting moiety, a bridging molecule to a ubiquitin E3 ligase complex, an E2 ubiquitin conjugating enzyme targeting molecule, an autophagy-targeting chimera, or a proteasome subunit targeting molecule;
wherein when R₁₄ is present, d is 1 or 2; and
wherein when R₁₃ is present, e is 1 or 2;
wherein R₁₁ comprises a substituted or unsubstituted bicyclic ring or a two-ring system, the bicyclic ring or two-ring system having 9 or 10 members with a carbonyl group at an ortho position to the alkene;
wherein each instance of R₁₂ independently is hydrogen, halogen, hydroxyl, trifluoromethyl, C1-C6 alkylamino, C1-C6 alkoxy, substituted or unsubstituted C1-C6 alkyl, a substituted or unsubstituted phenyl group, or Formula II, and wherein b is from 1 to 5;
wherein each instance of R₁₅ independently is hydrogen, halogen, hydroxyl, trifluoromethyl, C1-C6 alkylamino, C1-C6 alkoxy, or substituted or unsubstituted C1-C6 alkyl, or Formula II; and wherein c is from 1 to 5;
wherein R₁₆ is hydrogen, halogen, hydroxyl, trifluoromethyl, substituted or unsubstituted C1-C6 alkylamino, substituted or unsubstituted C1-C6 alkyl, an azide group, or Formula II; and
wherein R₁₇ is hydrogen or C1-C6 cyclic or linear alkyl.

Aspect 31. The compound of aspect 30, wherein R₁₁ is selected from:

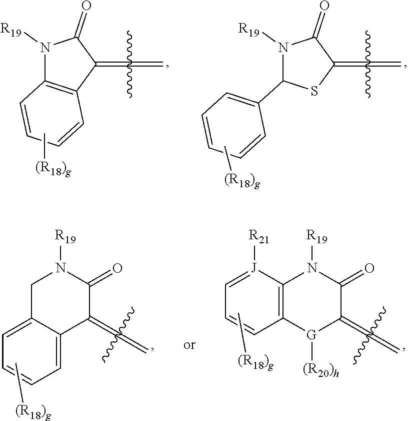

wherein G is C or S;
   wherein, when G is C, h is 2 or wherein, when G is S, h is 0;
   wherein each R₂₀ is independently selected from H, C1-C4 alkyl, or C3-C6 cycloalkyl;
wherein J is N or C;
   wherein, when J is N, R₂₁ is absent or, wherein, when J is C, R₂₁ is H;
wherein R₁₉ is selected from H, substituted or unsubstituted C1-C4 alkyl, or substituted or unsubstituted C3-C6 cycloalkyl or heterocycloalkyl; and
wherein each R₁₈ is independently selected from H, halogen, substituted or unsubstituted C1-C4 alkyl, C1-C6 alkoxy, substituted or unsubstituted C3-C6 cycloalkyl or heterocycloalkyl, —COOH, —OCF₃, —CF₃, or CN, and wherein g is from 1 to 5.

Aspect 32. The compound of aspect 31, wherein R₁₉ is selected from methyl, cyclopropyl, H, or

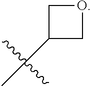 [SMILES: *C1COC1]

Aspect 33. The compound of aspect 31 or 32, wherein R₁₃ is selected from H, —COOH, —OCF₃, —CF₃, CN, methyl, cyclopropyl, or

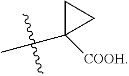 [SMILES: *C1(C(=O)O)CC1]

Aspect 34. The compound in any one of aspects 31-33, wherein T is nitrogen, U is carbonyl, and R₁₃ and R₁₄ are absent.

Aspect 35. The compound in any one of aspects 31-34, wherein D is phenyl.

Aspect 36. The compound in any one of aspects 31-35, wherein V is phenyl.

Aspect 37. The compound in any one of aspects 31-36, wherein R₁₅ is C1-C6 alkoxy.

Aspect 38. The compound in any one of aspects 31-37, wherein R₁₆ is Formula II.

Aspect 39. The compound in any one of aspects 31-38, wherein R₁₅ is C1-C6 alkoxy and R₁₆ is Formula II.

Aspect 40. The compound of aspect 30, having a structure represented by a formula:

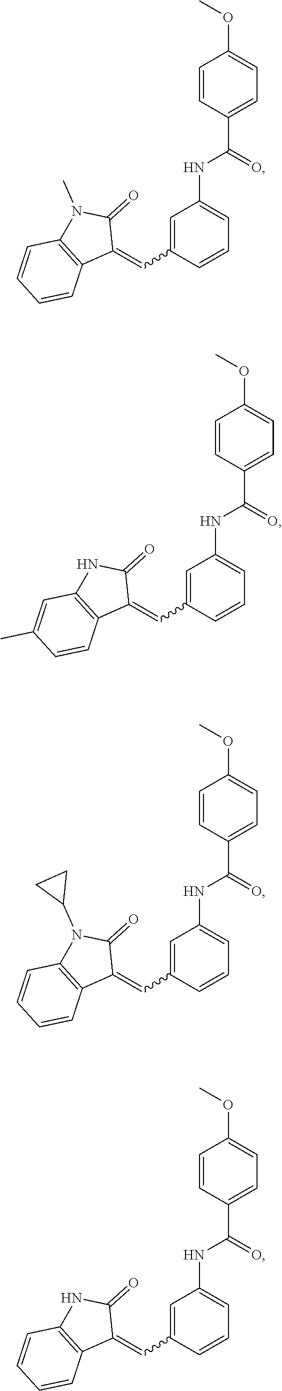 [SMILES: COc1ccc(C(=O)Nc2cccc(C=C3C(=O)N(C)c4ccccc43)c2)cc1 ; COc1ccc(C(=O)Nc2cccc(C=C3C(=O)Nc4cc(C)ccc43)c2)cc1 ; COc1ccc(C(=O)Nc2cccc(C=C3C(=O)N(C4CC4)c4ccccc43)c2)cc1 ; COc1ccc(C(=O)Nc2cccc(C=C3C(=O)Nc4ccccc43)c2)cc1]

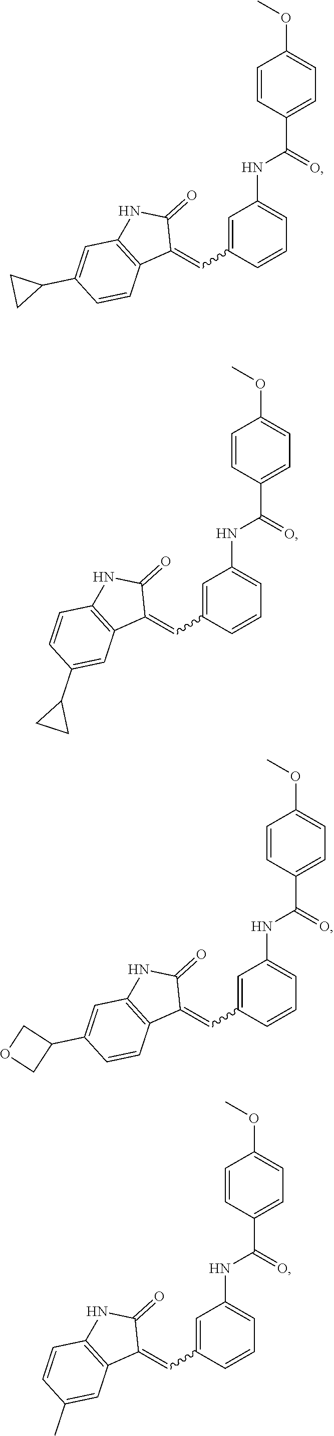 [SMILES: COc1ccc(C(=O)Nc2cccc(C=C3C(=O)Nc4cc(C5CC5)ccc43)c2)cc1 ; COc1ccc(C(=O)Nc2cccc(C=C3C(=O)Nc4ccc(C5CC5)cc43)c2)cc1 ; COc1ccc(C(=O)Nc2cccc(C=C3C(=O)Nc4cc(C5COC5)ccc43)c2)cc1 ; COc1ccc(C(=O)Nc2cccc(C=C3C(=O)Nc4ccc(C)cc43)c2)cc1]

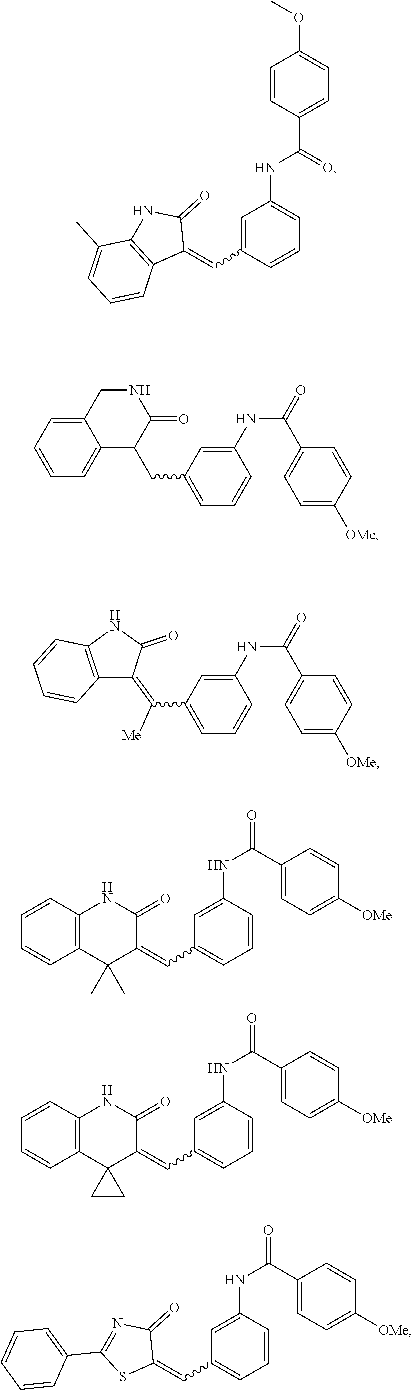 [SMILES: COc1ccc(C(=O)Nc2cccc(C=C3C(=O)Nc4c(C)cccc43)c2)cc1 ; COc1ccc(C(=O)Nc2cccc(CC3C(=O)NCc4ccccc43)c2)cc1 ; COc1ccc(C(=O)Nc2cccc(C(C)=C3C(=O)Nc4ccccc43)c2)cc1 ; COc1ccc(C(=O)Nc2cccc(C=C3C(=O)Nc4ccccc4C3(C)C)c2)cc1 ; COc1ccc(C(=O)Nc2cccc(C=C3C(=O)Nc4ccccc4C34CC4)c2)cc1 ; COc1ccc(C(=O)Nc2cccc(C=C3SC(c4ccccc4)=NC3=O)c2)cc1]

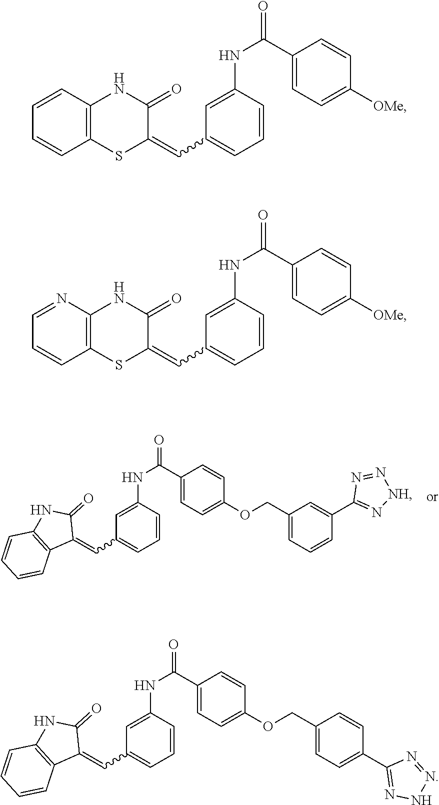 [SMILES: COc1ccc(C(=O)Nc2cccc(C=C3Sc4ccccc4NC3=O)c2)cc1 ; COc1ccc(C(=O)Nc2cccc(C=C3Sc4cccnc4NC3=O)c2)cc1 ; O=C1Nc2ccccc2C1=Cc1cccc(NC(=O)c2ccc(OCc3cccc(-c4nn[nH]n4)c3)cc2)c1 ; O=C1Nc2ccccc2C1=Cc1cccc(NC(=O)c2ccc(OCc3ccc(-c4nn[nH]n4)cc3)cc2)c1]

Aspect 41. The compound of aspect 30, having a structure represented by a formula selected from

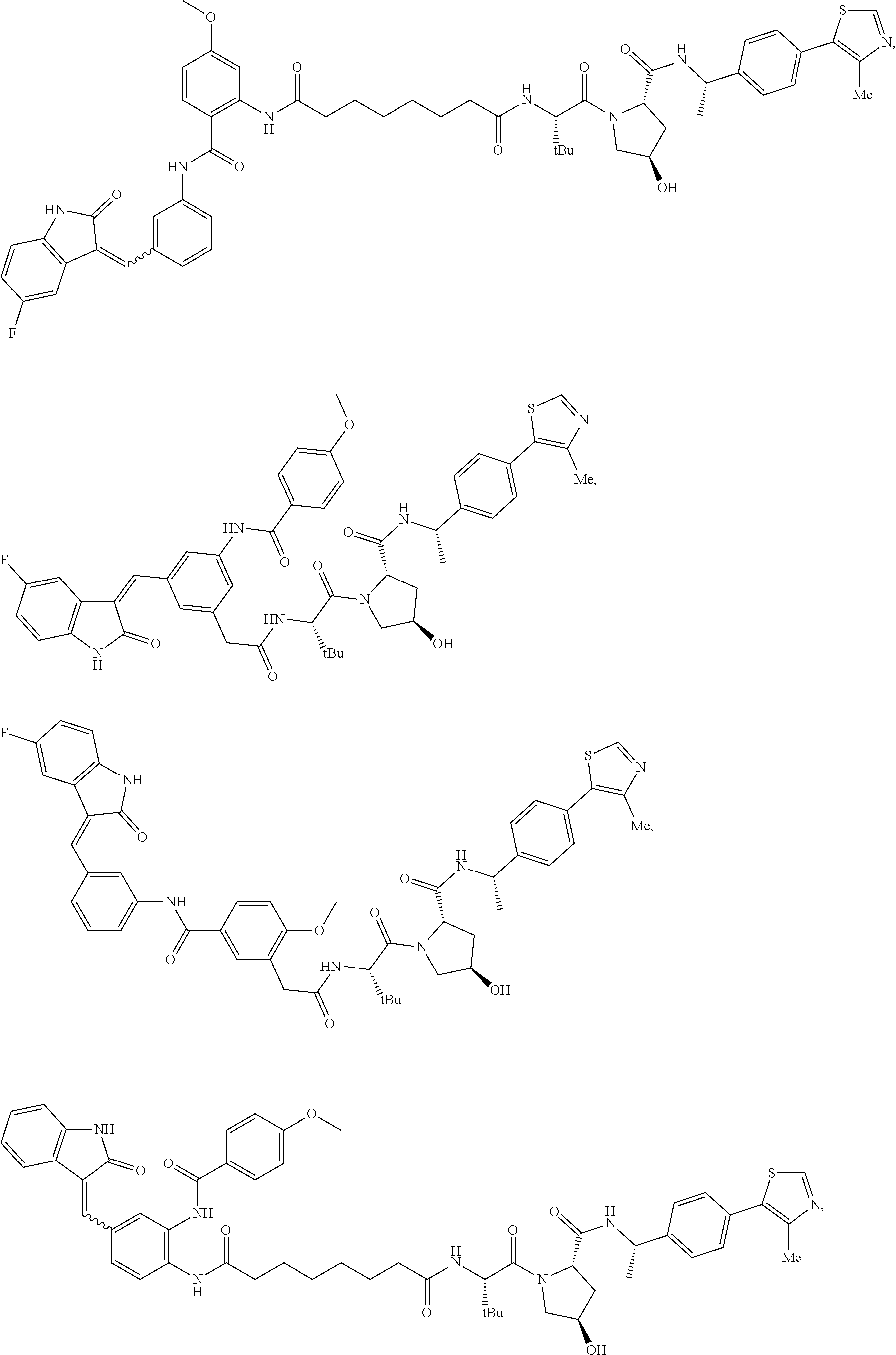

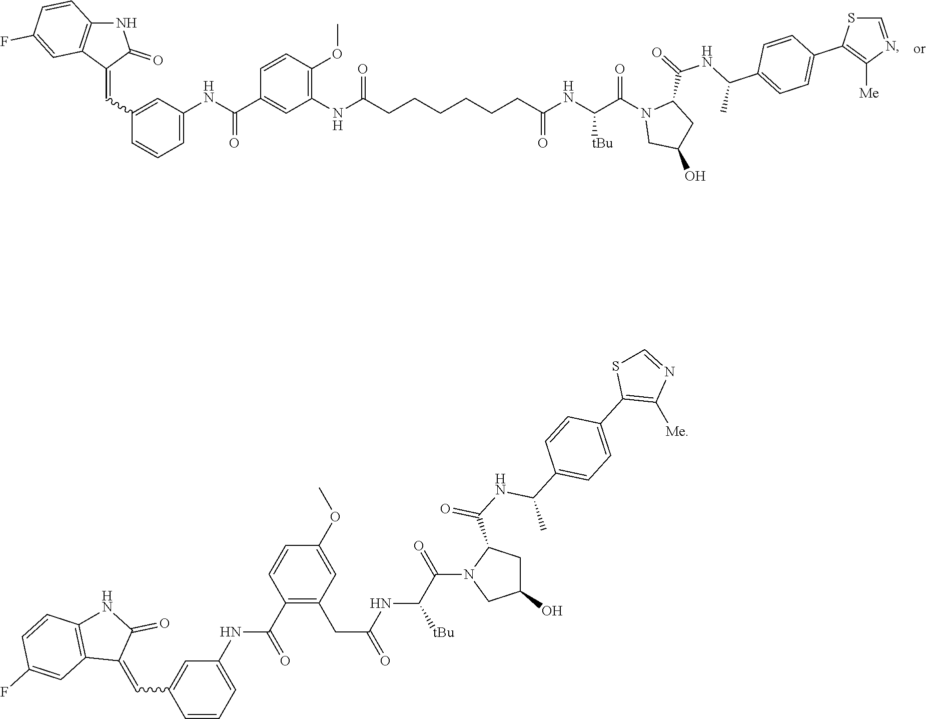 [SMILES: COc1ccc(C(=O)Nc2cccc(C=C3C(=O)Nc4ccc(F)cc43)c2)cc1NC(=O)CCCCCCC(=O)N[C@H](C(=O)N1C[C@H](O)C[C@H]1C(=O)N[C@@H](C)c1ccc(-c2scnc2C)cc1)C(C)(C)C ; COc1ccc(C(=O)Nc2cccc(C=C3C(=O)Nc4ccc(F)cc43)c2)c(CC(=O)N[C@H](C(=O)N2C[C@H](O)C[C@H]2C(=O)N[C@@H](C)c2ccc(-c3scnc3C)cc2)C(C)(C)C)c1]

Aspect 42. The compound of any one of aspects 1-41, further comprising a fluorescent label.

Aspect 43. The compound of aspect 42, wherein the fluorescent label comprises Cy5, Cy7, Alexafluor, BODIPY, rhodamine, or any combination thereof.

Aspect 44. A pharmaceutical composition comprising a therapeutically effective amount of a compound of any one of aspects 1-43, or a pharmaceutically acceptable salt, solvate, or polymorph thereof, and a pharmaceutically acceptable carrier.

Aspect 45. A method for detecting a cancer associated with RPN13, a truncated RPN13 containing an N-terminal Pleckstrin-like receptor for ubiquitin domain (RPN13-Pru), or a variant thereof in a subject, the method comprising:
(a) administering the compound of aspect 42 or 43 to the subject, wherein the compound localizes with the cancer; and
(b) quantifying fluorescence in a sample collected from the subject.

Aspect 46. The method of aspect 45, wherein the cancer is selected from multiple myeloma, lymphoma, mantle cell lymphoma, acute leukemia, cancers associated with human papillomavirus, colorectal cancer, gastric cancer, ovarian cancer, liver cancer, breast cancer, cervical cancer, pancreatic cancer, prostate cancer, or a combination thereof.

Aspect 47. A method for treating cancer in a subject, comprising the step of administering to the subject a therapeutically effective amount of at least one compound of any one of aspects 1-43, or a pharmaceutically acceptable salt thereof, or the pharmaceutical composition of aspect 44.

Aspect 48. The method of aspect 47, wherein the subject is a human.

Aspect 49. The method of aspect 47 or 48, wherein the cancer is selected from multiple myeloma, lymphoma, mantle cell lymphoma, acute leukemia, cancers associated with human papillomavirus, colorectal cancer, gastric cancer, ovarian cancer, liver cancer, breast cancer, cervical cancer, pancreatic cancer, prostate cancer, or a combination thereof.

Aspect 50. A method for inhibiting the activity of RPN13, RPN13-Pru, or a variant thereof in a subject, comprising the step of administering to the subject a therapeutically effective amount of at least one compound of any one of aspects 1-43, or a pharmaceutically acceptable salt thereof, or the pharmaceutical composition of aspect 44.

Aspect 51. The method of aspect 50, wherein the subject is a human.

Aspect 52. The method of any one of aspects 39-43, further comprising administering to the subject an agent known to decrease the activity of RPN13, RPN13-Pru, or a variant thereof.

Aspect 53. The method of any one of aspects 47-52, further comprising administering an anti-cancer agent to the subject.

Aspect 54. The method of aspect 52 or 53, wherein the anti-cancer agent or the agent known to decrease the activity or RPN13, RPN13-Pru, or a variant thereof comprises carfilzomib, bortezomib, ixazomib, disulfiram, marizomib, oprozomib, epoxomicin, MG132, KZR—616, KZR—504, PKS2279, PKS2252, or any combination thereof.

Aspect 55. A method for detecting a cancer in a subject, the method comprising:
(a) measuring a RPN13, RPN13-Pru, or a variant thereof biomarker in a sample from the subject to determine presence, absence, or a level of the biomarker; and
(b) correlating the measurement of the presence, absence, or level of the biomarker to the cancer.

Aspect 56. The method of aspect 55, wherein the cancer is selected from multiple myeloma, lymphoma, mantle cell lymphoma, acute leukemia, cancers associated with human papillomavirus, colorectal cancer, gastric cancer, ovarian cancer, liver cancer, breast cancer, cervical cancer, pancreatic cancer, prostate cancer, or a combination thereof.

Aspect 57. The method of aspect 55 or 56, wherein the sample comprises blood, serum, plasma, or a solid tissue sample.

Aspect 58. The method of any one of aspects 55-57, wherein the RPN13, RPN13-Pru, or a variant thereof biomarker is measured using mass spectrometry.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure.

### Example 1: Materials

XL5 and XL23 (Enamine ID Z44395249) were ordered from Enamine; XL5-¹³C₆-BA, XL24, XL28 and XL29 were obtained by customized synthesis from Enamine; XL5-¹³C₆-CB, XL25, XL26, XL27, XL30, XL31, XL32, XL33, XL5-VHL, XL5-VHL-2, X15-CRBN, XL5-IAP, VHL-Ac, IAP-Bz were synthesized according to reported literature procedures.

### Example 2: Structure-Based Screen Finds an hRpn13-Binding Compound

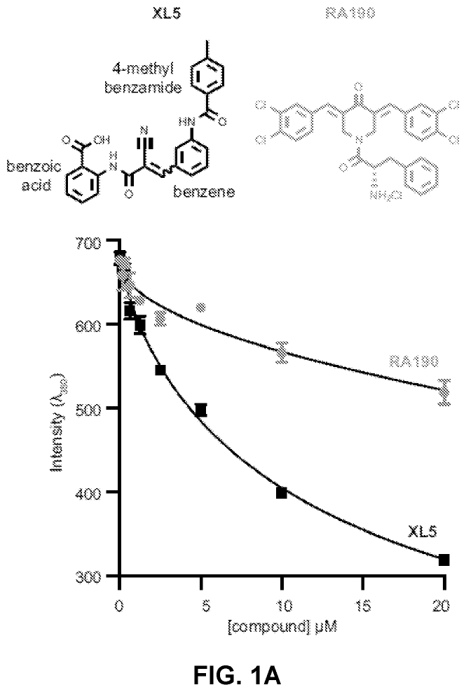

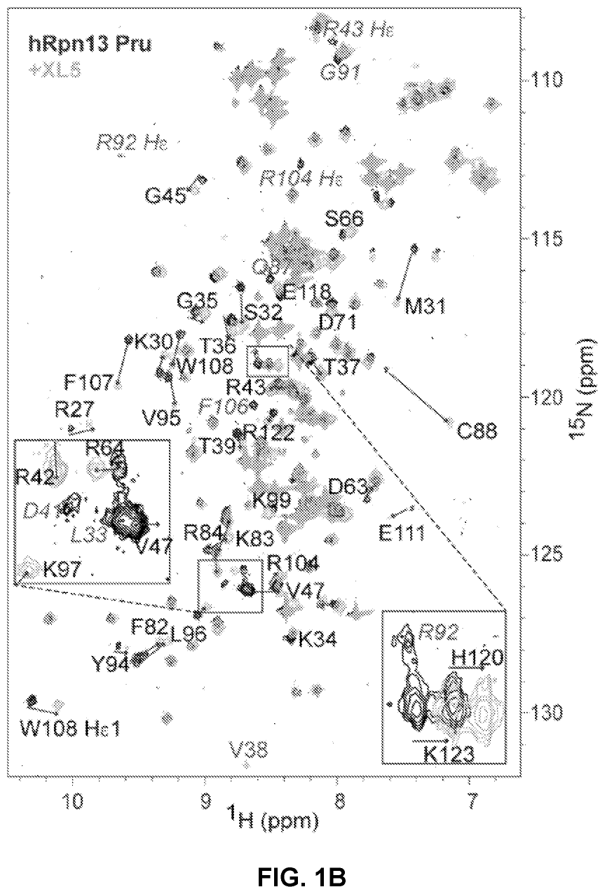

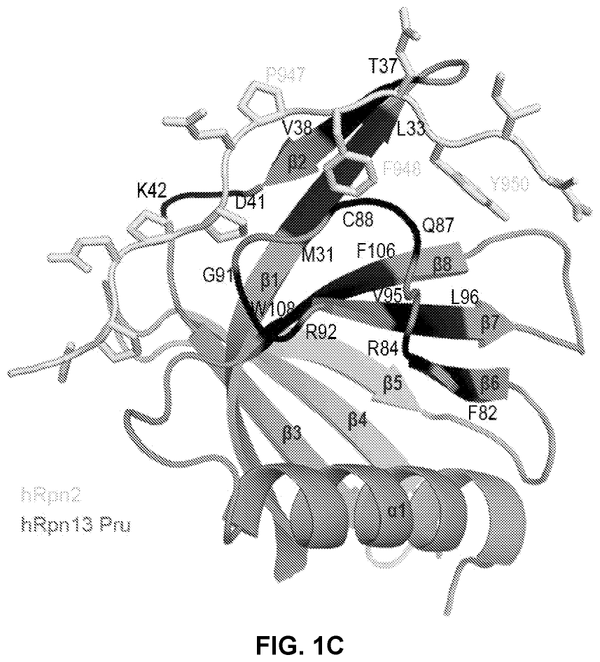

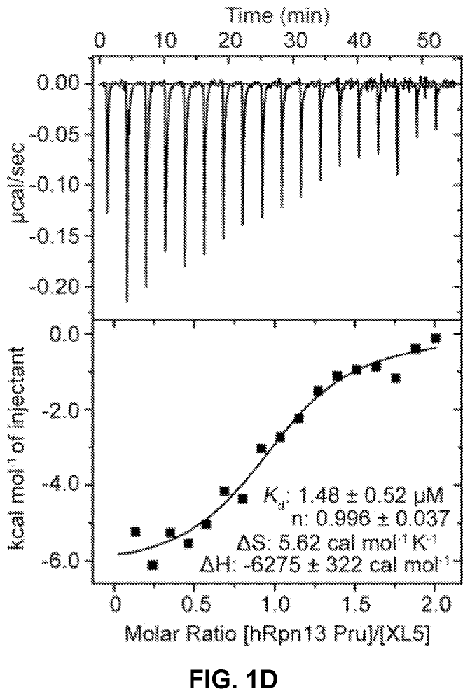

We conducted in silico docking screens of commercial libraries containing in total 63 million compounds by using the hRpn13 Pru:hRpn2 structure and hRpn2-binding site of hRpn13 as a binding pocket. Twenty-two potential lead compounds were selected for validation by biophysical assays. hRpn13 W108 is partially exposed when free but buried by hRpn2, enabling tryptophan quenching by differential scanning fluorimetry (DSF at λ₃₅₀) for assaying binding and this approach was used to screen for compound binding to this site. 20 μM compound (separately for twenty compounds) was incubated with 1 μM hRpn13 Pru and fluorescence emission at 350 nm measured. Greatest tryptophan quenching was observed by XL5 addition and incremental titration of XL5 into 1 μM hRpn13 Pru revealed concentration dependency (FIG. 1A). Eleven candidate compounds, including XL5, were evaluated further by NMR; XL4, which demonstrated tryptophan quenching, was excluded by insolubility at the required concentration. The compounds were separately added at 10-fold molar excess to 20 μM ¹⁵N-labeled hRpn13 Pru and binding assessed at 25° C. by 2D NMR for samples dissolved in NMR buffer (20 mM sodium phosphate, 50 mM NaCl, 2 mM DTT, 10% DMSO-d₆ (deuterated DMSO), pH 6.5). XL5 and no other tested compound indicated binding to hRpn13 by 2D NMR. XL5 addition caused hRpn13 signals to shift from free state positions to an observable bound state whereas spectral changes were not induced by the other compounds tested. Binding was also observed at 10° C. with XL5 at 2-fold molar excess and hRpn13 at 0.25 mM (FIG. 1B); this lower temperature leads to greater sample stability and was therefore used for the NMR experiments described below.

Consistent with the tryptophan quenching detected by DSF (FIG. 1A), XL5 caused the epsilon and amide signals for W108 to shift (FIG. 1B). We quantified the shifting of the NMR signals following XL5 addition across the hRpn13 sequence to identify all significantly affected amino acids. In some cases, signals appear or disappear, such as the V38 amide signal, which appears upon XL5 addition, or the amide signals for L33, D41, Q87, G91, R92, and F106 and epsilon signals for R43, R92, and R104, all of which disappear following XL5 addition (FIG. 1B). We mapped the hRpn13 amino acids most affected by XL5 onto a ribbon diagram of hRpn2-bound hRpn13 Pru (PDB 6C₀₄). The affected amino acids center around the region bound by hRpn2 F948 (FIG. 1C), which is required for hRpn2 binding to hRpn13. We used isothermal titration calorimetry (ITC) to measure the binding affinity between hRpn13 and XL5. hRpn13 Pru was added incrementally to XL5 and the data fit to a 1-site binding mode (FIG. 1D). An overall binding affinity (K_{d}) of 1.48±0.52 μM was determined with favorable enthalpy and entropy. We attempted to measure the binding affinity of RA190 for the hRpn13 Pru by ITC but did not detect binding by this method, which relies on measurement of enthalpy changes (heat effects). We were able to detect tryptophan fluorescence emission quenching following RA190 addition to hRpn13 Pru, with a titration-dependent reduction in λ₃₅₀ signal (FIG. 1A).

We modified various functional groups of the XL5 chemical scaffold, including the 4-methyl benzamide (R1, R2, R3, X), benzoic acid (R4, R5, R6) and central benzene (R7, R8) groups. Each modification yielded a compound with either equivalent or reduced affinity for hRpn13 Pru, as assessed by ITC and NMR.

### In Silico Screening Methods

Docking screens were conducted with the ICM-Pro (Molsoft LCC) software by running up to 1000 parallel processes on 6000 CPUs of the National Institutes of Health Biowulf cluster supercomputer. For the initial screens, the entire hRpn2-binding cleft of hRpn13 was used, including all hRpn13 residues in contact with hRpn2 (940-953), as defined by the NMR and x-ray structures. These amino acids were defined as the targeted binding pocket. Libraries ranged in size from 0.6 to 40 million compounds that were either commercially available (Enamine diversity set, Emolecules, Mcules, Asinex, UORSY, Chembridge, ChemDiv, ChemSpace) or capable of synthesis (Enamine's diversity REAL database containing 15 million compounds). In total, 63 million compounds were screened. Most of the hits targeted the pocket occupied by the C-terminal end of hRpn2. Enamine's diversity library of 1.92 million compounds demonstrated the highest hit rate with 5,155 compounds identified in a preliminary fast screen run with a thoroughness value of 1. Hits from the first screens were subjected to more thorough and slow automatic docking with a thoroughness value of 100. 20-30 top compounds from the second round of screens were redocked manually and the best scoring compounds selected for ordering/synthesis and experimental testing.

### Differential Scanning Fluorimetry Experiments

DSF experiments were performed on a PrometheusNT.48 instrument (NanoTemper Technologies, Germany) at 20° C. 40 μM compound was added to equal volume of 2 μM hRpn13 Pru in buffer C (20 mM sodium phosphate, 50 mM NaCl, 10% DMSO, pH 6.5). For FIG. 1A, 2 μM hRpn13 Pru was added to equal volume of serially diluted XL5 or RA190 in buffer C. Each sample was loaded into three high sensitivity capillaries (NanoTemper, cat #PR-C006) and the emission of intrinsic tryptophan fluorescence at 350 nm was monitored.

### Isothermal Titration Calorimetry Experiments

ITC experiments were performed at 25° C. on a MicroCal iTC200 system (Malvern, PA, USA). hRpn13 Pru, XL5, XL5 derivative, or RA190 were prepared in buffer C. One aliquot of 0.5 μL followed by 17 or 18 aliquots of 2.1 μL of 200 μM hRpn13 Pru was injected at 750 r.p.m. into a calorimeter cell (volume 200.7 ml) that contained 20 μM XL5, XL5 derivative, or RA190. Blank experiments were performed by replacing XL5, XL5 derivative, or RA190 with buffer in the cell and the resulting data subtracted from the experimental data during analyses. The integrated interaction heat values were normalized as a function of protein concentration and the data were fit with MicroCal Origin 7.0-based software implementing the “One Set of Sites” model to yield binding affinity Kₐ (1/K_{d}), stoichiometry, and other thermodynamic parameters. Dissociation constants (K_{d}) as determined by ITC as well as relative binding affinities of selected compounds are presented n Table 1, while additional information on named compounds including structures, synthesis, and characterization is presented in Examples 3-9:

TABLE 1
Binding Affinities of Selected Compounds to hRpn13-Pru

| Compound | Relative Binding Affinity (NMR) | ITC K_{d} (μM) |
|---|---|---|
| XL5 | ++++++ | 1.48 ± 0.52 |
| XL23 | ++++ | 3.88 ± 0.43 |
| XL24 | +++++ | 1.74 ± 0.35 |
| XL25 | +++++ | 4.12 ± 1.47 |
| XL26 | +++++ | 6.67 ± 1.97 |
| XL27 | +++++ | 3.94 ± 1.02 |
| XL28 | ++++ | 3.82 ± 0.26 |
| XL29 | ++++ | 7.81 ± 1.28 |
| XL30 | ++ | 12.39 ± 5.91 |
| XL31 | + | NA |
| XL32 | +++ | NA |
| XL33 | + | NA |

### Example 3: XL5 Binds Covalently to hRpn13 Pru

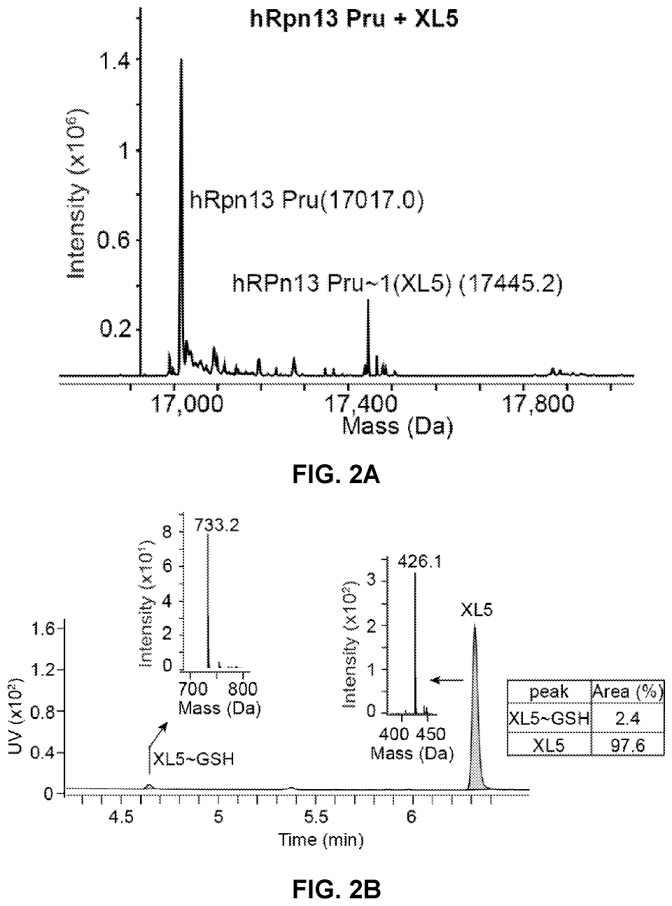

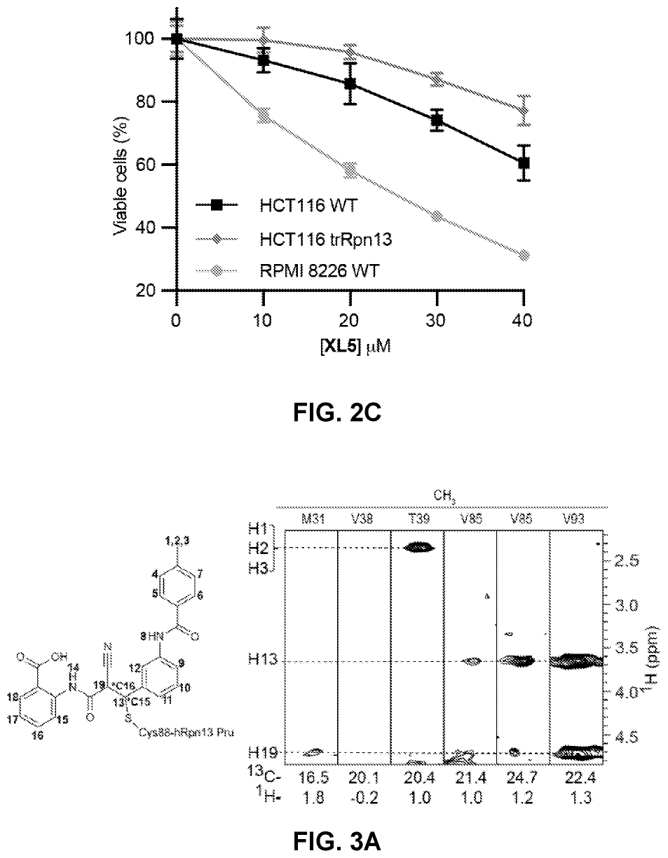

Model structures predicted from the in silico screen indicate XL5 to bind non-covalently to hRpn13 at a location somewhat different from that suggested by the experimental data. hRpn13 C88 demonstrated shifting in 2D NMR spectra following XL5 addition (FIG. 1B) and this finding combined with the presence of an α,β-unsaturated carbonyl in XL5 led us to hypothesize that, like RA190, XL5 may interact with hRpn13 by Michael addition at C88 (FIG. 1A). To test for covalent interaction, an hRpn13 Pru sample was incubated with 10-fold molar excess XL5 or DMSO (as a vehicle control) and subjected to liquid chromatography-mass spectrometry (LC-MS). A product was detected of appropriate molecular weight for covalent addition of XL5 to hRpn13 Pru (FIG. 2A), which was absent from the control experiment. To test for general reactivity of XL5 towards exposed cysteines, 40 μM XL5 was incubated at 4° C. for two hours with 2 mM reduced L-glutathione serving as representative of a non-specific interactor with exposed cysteines. XL5-ligated glutathione was detected at only 2% abundance (FIG. 2B). Under identical conditions, 40 μM RA190 reacted with 2 mM reduced L-glutathione to yield products with one or two molecules ligated at 14% or 30% abundance, respectively. We also tested XL5 reactivity by incubating it at 0.2 μM with mouse serum (BioIVT) and monitoring the effect by LC-MS over a 24-hour time period to find only 6% reduction in the unligated compound.

Sample Preparation of hRpn13 and hRpn2

hRpn13 Pru (1-150) or hRpn2 (940-953) was expressed in E. coli BL21(DE3) pLysS cells (Invitrogen) as a recombinant protein in frame with an N-terminal histidine tag or glutathione S-transferase respectively followed by a PreScission protease cleavage site. Cells were grown at 37° C. to optical density at 600 nm of 0.6 and induced for protein expression by addition of isopropyl-β-D-thiogalactoside (0.4 mM) for 20 hours at 17° C. or 4 hours at 37° C. The cells were harvested by centrifugation at 4,550 g for 40 min, lysed by sonication, and cellular debris removed by centrifugation at 31,000 g for 30 min. The supernatant was incubated with Talon Metal Affinity resin (Clontech) for one hour or Glutathione S-sepharose 4B (GE Healthcare Life Sciences) for 3 hours and the resin washed extensively with buffer A (20 mM sodium phosphate, 300 mM NaCl, 10 mM @ME, pH 6.5). hRpn13 Pru was eluted from the resin by overnight incubation with 50 units per mL of PreScission protease (GE Healthcare Life Sciences) in buffer B (20 mM sodium phosphate, 50 mM NaCl, 2 mM DTT, pH 6.5) whereas GST-hRpn2 (940-953) was eluted in buffer B containing 20 mM reduced L-glutathione. The eluent was subjected to size exclusion chromatography with a Superdex75 column on an FPLC system for further purification. ¹⁵N ammonium chloride and ¹³C glucose were used for isotopic labeling.

### LC-MS Experiments

LC-MS experiments were performed on a 6520 Accurate-Mass Q-TOF LC/MS system equipped with a dual electro-spray source, operated in the positive-ion mode. Samples included 2 μM hRpn13 Pru incubated for 2 hours at 4° C. with 10-fold molar excess XL5 in buffer C containing 0.2% DMSO as well as 2 mM reduced L-glutathione incubated for 2 hours at 4° C. with 40 μM XL5 or RA190 in buffer C containing 0.4% DMSO. Acetonitrile was added to all samples to a final concentration of 10%. Data acquisition and analysis were performed by Mass Hunter Workstation (version B.06.01). For data analysis and deconvolution of mass spectra, Mass Hunter Qualitative Analysis software (version B.07.00) with Bioconfirm Workflow was used.

To check for reactivity of XL5 in mouse serum, 0.2 μM XL5 was mixed with mouse serum (BioIVT) and aliquots of the spiked mixture left at room temperature for 0, 4, 8 and 24 hours. For each time point, six samples were extracted using 75% acetonitrile and 0.075% formic acid. The supernatant was transferred to polypropylene injection vials for LC-MS analysis. LC-MS was performed with a TSQ Quantiva triple quadrupole mass spectrometer (Thermo Fisher Scientific) operating in selected reaction monitoring mode with positive electrospray ionization and with a Shimadzu 20AC-XR system using a 2.1×50 mm, 2.7 μm Waters Cortecs C18 column.

### Example 4: XL5 Treatment Causes Reduced Cell Viability and Apoptosis

Since previous hRpn13-targeting molecules induce apoptosis in multiple myeloma and colon cancer cells we tested whether XL5 restricts the RPMI 8226 multiple myeloma and HCT116 colon cancer cell lines by measuring metabolism with an MTT (3-(4,5-dimethylthiazol-2-yl)-2,5-diphenyltetrazolium bromide) assay. Experiments were also conducted in parallel with a HCT116 colon cancer cell line in which the exon encoding hRpn13 C₈₈ is deleted, named trRpn13; this cell line expresses a truncated hRpn13 protein with a defective Pru and inability to bind the proteasome28. RPMI 8226 and WT (wild-type) or trRpn13 HCT116 cells seeded at 8,000 and 4,000 cells per well were treated with varying concentrations of XL5 extending to 40 μM and compared to cells incubated with equivalent amounts of DMSO vehicle control. Reduced metabolic activity was observed with XL5 treatment in a concentration-dependent manner in all cell lines but with a more pronounced effect in RPMI 8226 cells (FIG. 2C). Moreover, the potency of XL5 was reduced in HCT116 trRpn13 cells compared to HCT116 WT cells (FIG. 2C).

### Cell Culture and Antibodies

The HCT116 WT (ATCC®CCL-247™), RPMI 8226 (ATCC® CCL-155™) and 293T (ATCC® CRL-3216™) cell lines were purchased from the American Tissue Culture Collection; HCT116 trRpn13 cells were generated and described as part of a previous study. HCT116, RPMI 8226 or 293T cell lines were grown in McCoy's 5A modified (Thermo Fisher Scientific 16600082), RPMI-1640 (ATCC® 30-2001™) or DMEM (Thermo Fisher Scientific, 10569010) media supplemented with 10% fetal bovine serum (Atlanta Biologicals) and in a 37° C. humidified atmosphere of 5% CO₂. Antibodies (dilutions) used in this study include anti-hRpn13 (Abcam ab157185, 1:5,000), anti-hRpn2 (Abcam ab2941, 1:1,000), anti-hRpt3 (Abcam ab140515, 1:1,000) anti-β-actin (Cell Signaling Technology 4970s or 3700s, 1:3,000 or 1:5,000), anti-cleaved caspase-9 (Cell Signaling, 52873s, 1:1,000), anti-GST (Cell Signaling, 2625s, 1:10,000)) anti-mouse (Sigma-Aldrich, 1:3,000 or 1:4,000), anti-rabbit (Life Technologies, A16110, 1:5,000, 1:10,000 or 1:20,000) and anti-native rabbit (Sigma-Aldrich, 1:1000) antibodies.

### MTT Assay

HCT116 WT or trRpn13 cells were seeded at 4,000 cells/well whereas RPMI 8226 cells were seeded at 8,000 cells/well with RPMI 1640 medium (no phenol red, Thermo Fisher Scientific 11835030) containing 2% fetal bovine serum in 96-well plates. Cells were treated with 0.4% DMSO (as a control) and this concentration was maintained with XL5, XL5-PROTACs XL5-VHL, XL5-VHL-2, XL5-CRBN, or XL5-IAP, and E3 ligands VHL-Ac, thalidomide (Selleckchem, catalog NO. S1193), or IAP-Bz at 10 μM, 20 μM, 30 μM or 40 μM concentration. After 48 hours, 0.35 mg/mL MTT was added for 4 hours of incubation. Stop solution (40% DMF, 10% SDS (W/V), 25 mM HCl, 2.5% acetic acid in H₂O) was added to the cells and incubated overnight. Absorbance at 570 nm was measured by using CLARIOstar (BMG LABTECH).

### XL5 Treatment

HCT116 WT or trRpn13 cells and RPMI 8226 cells were treated with 40 μM or 100 μM XL5, 40 μM XL5-PROTACs or 0.4% or 0.8% DMSO (as a control) for 18 or 24 hours, as indicated.

### Immunoblotting

HCT116 WT, HCT116 trRpn13, RPMI 8226 or 293T cells were collected and washed with PBS followed by lysing in 1% Triton-TBS lysis buffer (50 mM Tris-HCl, pH 7.5, 150 mM NaCl, 1 mM PMSF) supplemented with protease inhibitor cocktail (Roche). Total protein concentration was determined by bicinchoninic acid (Pierce). Protein lysates were prepared in 1χ LDS (ThermoFisher, NP0007) buffer with 100 mM DTT and heating at 70° C. for 10 min, loaded onto 4-12% Bis-Tris polyacrylamide gels (Life Technologies), subjected to SDS-PAGE and transferred to Invitrolon polyvinylidene difluoride membranes (Life Technologies). The membranes were blocked in Tris-buffered saline with 0.1% Tween-20 (TBST) supplemented with 5% skim milk, incubated with primary antibody, washed in TBST, incubated with secondary antibody and washed extensively in TBST. Pierce™ ECL Western Blotting Substrate (32106; Thermo Fisher Scientific) or Amersham™ ECL™ Primer Western Blotting Detection Reagent (cytiva) was used for antibody signal detection.

### Immunoprecipitation

RPMI 8226 cell lysates (1 mg) were incubated with anti-hRpt3 or IgG (rabbit) antibodies overnight at 4° C. and then incubated for an additional 3 hours at 4° C. with 50 μL Dynabeads™ protein G (Life Technologies, 10004D). Following three washes with 1% Triton-TBS lysis buffer, proteins bound to the Dynabeads™ protein G were eluted by using 2χ LDS with 100 mM DTT and analyzed by immunoblotting.

### GST-Pulldown Assay

RPMI 8226 cell lysates (2 mg) were incubated with 2 nmol GST or purified GST-hRpn2 (940-953) overnight at 4° C. and then incubated for an additional 3 hours at 4° C. with 25 μL pre-washed Glutathione Sepharose 4B resin (cytiva). Following three washes with 1% Triton-TBS lysis buffer, proteins bound to the Glutathione Sepharose 4B resin were eluted by using 2χ LDS with 100 mM DTT and analyzed by immunoblotting.

### Example 5: Structure of XL5-Ligated hRpn13

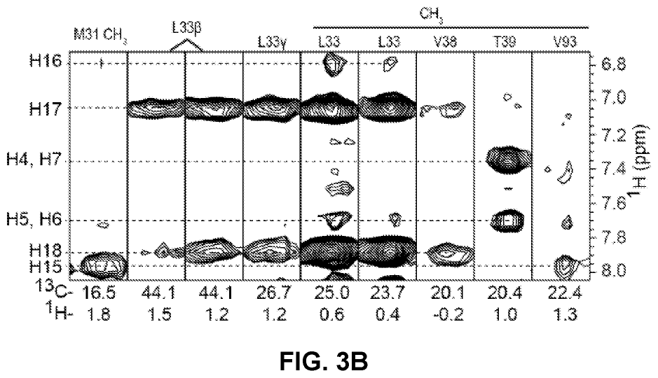

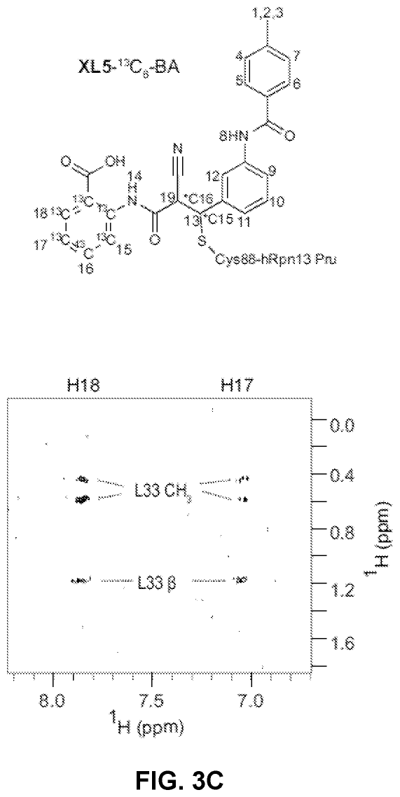

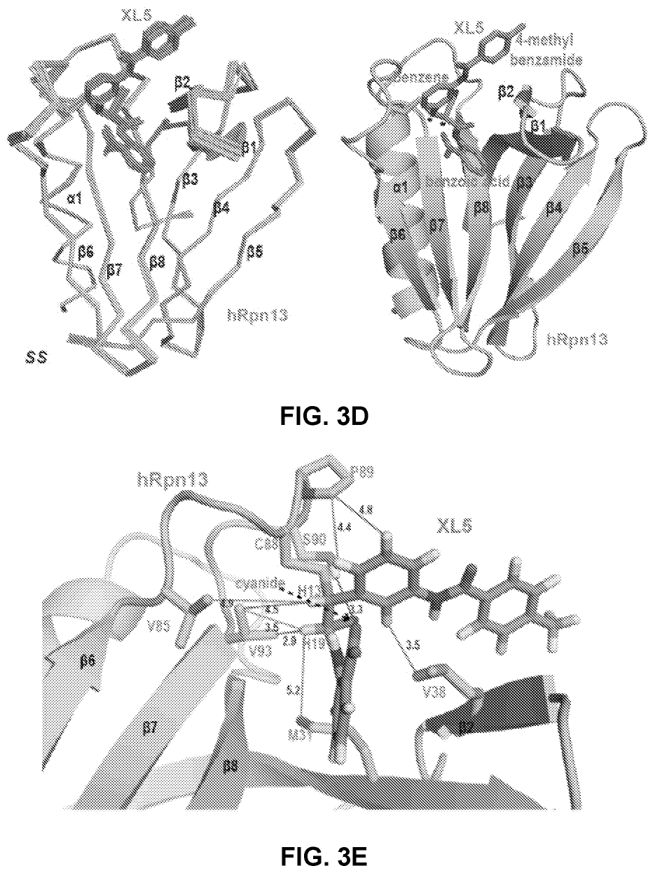

We used NMR to solve the structure of XL5-ligated hRpn13. Chemical shift values were assigned to hRpn13 and XL5 (FIGS. 3A-3B) as described in the Methods. A ¹³C-dispersed NOESY experiment recorded on ¹³C-labeled hRpn13 Pru mixed with 1.2-fold molar excess unlabeled XL5 revealed NOEs that demonstrated preservation of the hRpn13 Pru structure. A ¹H, ¹³C half-filtered NOESY experiment acquired on ¹³C-labeled hRpn13 Pru mixed with 2-fold molar excess unlabeled XL5 selectively recorded their interactions (FIGS. 3A-3B). Protons indicating saturation of the alkene group (H13 and H19 in FIG. 3A, left panel) were present in the spectra, forming NOE interactions with methyl groups of hRpn13 V85 and V93 (FIG. 3A), consistent with XL5 ligation to C88 of the p6-P7 loop. NOEs involving XL5 H15-H18 were also detected to hRpn13 methyl groups of M31, L33, V38 and V93 (FIG. 3B). These interactions were validated by selective ¹³C-labeling of the XL5 benzoic acid ring (FIG. 3C, XL5-¹³C₆-BA) in complex with equimolar hRpn13 Pru for hRpn13 L33 and XL5 H17 and H18 (FIG. 3C); the weaker interactions involving hRpn13 V38 as well as XL5 H15 and H16 (FIG. 3B) were not observable in this less sensitive experiment. Signals from H4 and H5 of the XL5 4-methyl benzamide group are indistinguishable compared to H7 and H6 respectively (FIG. 3A, left panel), but interactions were recorded between H4/H7 and H5/H6 of XL5 and hRpn13 T39 (FIG. 3B), which also exhibited NOE interactions with the XL5 methyl group (FIG. 3A). In total, the ¹H, ¹³C half-filtered NOESY experiments yielded 23 NOE interactions between hRpn13 and XL5 (FIGS. 3A-3B, Table 1).

When ligated to hRpn13 C₈₈, XL5 C₁₅ and C16 (FIG. 3A, left panel) can in principle adopt either R or S stereochemistry and we therefore initially calculated structures for XL5-ligated hRpn13 with all possible stereochemistry, including SS, RR, SR and RS for C15 and C16 respectively. Only SS stereochemistry fit the NOESY data. These calculated structures converged with a heavy atom root-mean-square-deviation (r.m.s.d.) of 0.54 Å (FIG. 3D, left panel). A key feature of XL5 interaction with hRpn13 is the sulfide bond formed to the C88 thiol group (FIG. 3D, right panel and FIG. 3E) facilitated by nearby interactions from XL5 H13 and H19 to hRpn13 M31, V85, and V93 methyl groups (FIG. 3E).

### NMR Experimental Methods

For screening by ¹H, ¹⁵N HSQC experiments, small molecule dissolved in DMSO-d₆ was added to 20 μM or 250 μM ¹⁵N-labeled hRpn13 Pru at a molar excess of 2-fold (for XL5) or 10-fold (for all compounds tested) in NMR buffer (20 mM sodium phosphate, 50 mM NaCl, 2 mM DTT, 10% DMSO-d₆, pH 6.5). All NMR experiments were conducted at 10° C. unless indicated to be at 25° C. and on Bruker Avance 600, 700, 800 or 850 MHz spectrometers equipped with cryogenically cooled probes. The ¹³C-edited NOESY spectrum was acquired with a 100 ms mixing time on a mixture of 0.4 mM ¹³C-labeled hRpn13 Pru and 0.48 mM unlabeled XL5 in NMR buffer containing 70% 2H₂O. Three ¹³C-half-filtered NOESY experiments were recorded with a 100 ms mixing time on asymmetrically labeled samples dissolved in NMR buffer. One sample contained 0.25 mM ¹³C-labeled hRpn13 Pru mixed with 2-fold molar excess unlabeled XL5; another contained 0.5 mM hRpn13 Pru and 0.5 mM XL5 with the central benzene ring ¹³C-labeled (XL5 ¹³C₆-CB); and a third contained 0.4 mM hRpn13 Pru and 0.4 mM XL5 with the benzoic acid ring ¹³C-labeled (XL5 ¹³C₆-BA) dissolved in NMR buffer containing 70% 2H₂O. An ¹⁵N-dispersed NOESY spectrum was acquired with a 120 ms mixing time on 0.25 mM ¹⁵N-labeled hRpn13 Pru mixed with 2-fold molar excess unlabeled XL5 dissolved in NMR buffer. The ¹H, ¹³C HMQC experiments were acquired on 0.5 mM XL5-¹³C₆-CB in NMR buffer with and without DTT as well as mixed with equimolar unlabeled hRpn13 Pru; a control experiment with only 0.5 mM hRpn13 Pru was also recorded in NMR buffer to assign natural abundance signals of hRpn13. [0346] 2D ¹³C-edited HCCH-TOCSY (12 ms mixing time), NOESY (500 ms mixing time), or ¹H, ¹³C HMQC spectra were recorded on 10 mM XL5-¹³C₆-BA in DMSO-d₆ at 25° C., and ¹H, ¹³C HMQC spectra were recorded in NMR buffer on 0.1 mM XL5-¹³C₆-BA with increasing molar ratio of unlabeled hRpn13 Pru, including at 1:0, 1:0.5, 1:1, 1:2, and 1:4. Data were processed by NMRPipe and visualized with XEASY.

### Chemical Shift Assignments

Chemical shift assignments for hRpn13 were aided by a previous study and confirmed by NOESY experiments; namely, an ¹⁵N-dispersed NOESY (120 ms mixing time) experiment recorded in NMR buffer on 0.25 mM ¹⁵N hRpn13 Pru mixed with 2-fold molar excess XL5 or a ¹³C-edited NOESY (100 ms mixing time) experiment recorded on a mixture of 0.48 mM unlabeled XL5 and 0.4 mM ¹³C labeled hRpn13 Pru dissolved in NMR buffer with 70% 2H₂O.

To aid in the chemical shift assignment of XL5, we selectively ¹³C-labeled either the benzoic acid aromatic ring or the central benzene ring; we refer to these samples as XL5-¹³C₆-BA and XL5-¹³C₆-CB respectively. H15, H16, H17 and H18 from XL5 were assigned by using ¹³C-edited 2D HCCH-TOCSY, 2D NOESY and HMQC spectra recorded on 10 mM XL5-¹³C₆-BA in DMSO-d₆. These assignments could be transferred for XL5 dissolved in NMR buffer although shifting and splitting was observed due to the presence of 2 mM DTT. Addition of unlabeled hRpn13 Pru caused shifting for XL5 H17 and H18, as well as the H15 and H16 signals to attenuate. Without DTT, the four expected signals for H9, H10, H11 and H12 appeared in the spectrum recorded on XL5-¹³C₆-CB; however, inclusion of DTT in the NMR buffer caused multiple new signals to appear, as was observed for the XL5 benzoic acid group. Addition of hRpn13 Pru caused all XL5-¹³C₆-CB signals present in the ¹H, ¹³C HMQC spectrum to disappear with the exception of one weak signal; this resonance was assigned to H12 by an NOE interaction to H8 of XL5 that was observed in a ¹H, ¹³C half-filtered NOESY experiment recorded on 0.5 mM XL5-¹³C₆-CB mixed with equimolar unlabeled hRpn13 Pru.

### Chemical Shift Perturbation

Chemical shift perturbation (CSP) analysis was done by comparing ¹H, ¹⁵N HSQC experiments recorded on ¹⁵N-labeled hRpn13 Pru alone and with 2-fold molar excess unlabeled XL5. CSP values were calculated according to Eq 1, where AbN and A⁶H symbolize change in amide and proton signal, respectively, and a threshold of one standard deviation above average was used for the plot (FIG. 1C).

$$C SP = {( 0.2 {( {Δδ}_{N} )}^{2} + {( {Δδ}_{H} )}^{2} )}^{1 / 2} \qquad ( 1 )$$

### Structure Determination

Distance, dihedral angle and hydrogen bond restraints were generated from the unligated hRpn13 Pru crystal structure (PDB 5IRS) with the exception of amino acids at the binding interface, including M31, L33, V38, T39, V85 V93 and F106, for which restraints from the spectra recorded on XL5-ligated hRpn13 were used exclusively to allow for rearrangements due to XL5 binding. These restraints were combined with 23 NOE-derived distance restraints between hRpn13 and XL5 (FIGS. 3A-3B, Table 1) to calculate the XL5-ligated hRpn13 Pru structure. The calculations were done by using simulated annealing algorithms in XPLOR-NIH 2.50 (http://nmr.cit.nih.gov/xplor-nih/). An initial set of topology and parameter files for the ligand were generated by PRODRG and corrected to require the angles in the planar 6-membered rings to sum to 360°. XL5 was covalently bonded to the hRpn13 C₈₈ sulfur of PDB 5IRS (as displayed in FIG. 3A) with chirality at XL5 C₁₅ and C16 of S, S (SS), R, R (RR), S, R (SR) or R, S (RS) stereochemistry. Each stereoisomer was used as a starting structure for iterative simulated annealing to generate 200 initial structures, from which twenty were chosen based on criteria of no NOE, dihedral or torsion angle violation and lowest energy. The structures were then clustered into converged sets and evaluated based on adherence to differential NMR data such that distances were closer for interacting protons with stronger NOEs. The only structures that fit all of the NMR data were those of SS stereochemistry and in the main cluster 1 which contained seventeen of the twenty calculated SS structures. This cluster places XL5 H17 closer to hRpn13 L33 Hy than XL5 H18 and XL5 H18 closer to a hRpn13 V38 methyl group than XL5 H17 and H15. These differential interactions are indicated by the stronger NOEs observed between XL5 H17 or H18 with hRpn13 L33 Hy or V38 methyl group respectively (FIGS. 3A-3B) and not preserved in cluster 2. The calculated RS and SR structures formed four clusters whereas the RR structures formed 6 clusters; however, these clusters failed to fit the NMR data, such as the directing of RS cluster 1 or SR cluster 3 XL5 H13 away from hRpn13 V85 (FIG. 3A) or yielding equivalent interactions for XL5 H19, H15, H17 or H18 with the observed hRpn13 V38 methyl group as occurs in RS cluster 2-4, SR cluster 1, 2 and 4, and RR cluster 1-4 (FIG. 3A). Similarly, the closer proximity in RR cluster 5 and cluster 6 of the hRpn13 V85 methyl groups to XL5 H19 than XL5 H13 is not supported by NMR data (FIG. 3A). Altogether, our structure calculations indicate that XL5 binds to hRpn13 with SS chirality for XL5 C₁₅ and C16.

A weak hydrogen bond between the hRpn13 S90 sidechain hydroxy group and XL5 cyanide group was found in eight of the SS cluster 1 structures. Therefore, this hydrogen bond was included as an additional distance restraint (Table 1) and a new iteration of SS structure calculations was performed to yield 20 final lowest energy structures without hRpn13 distance or dihedral angle violations greater than 0.5 Å or 5° respectively and no torsion angle violations. This final set of 20 structures was selected for visualization and statistical analyses. Structure evaluation was performed with the program PROCHECK-NMR; the percentage of residues in the most favored, additionally allowed, generously allowed and disallowed regions was 94.3, 5.7, 0.1 and 0.0, respectively. Visualization was performed with MOLMOL or PyMOL (PyMOL Molecular Graphics System, https://www.pymol.org/2/).

### Example 6: hRPN13 Targeting Mechanisms of XL5

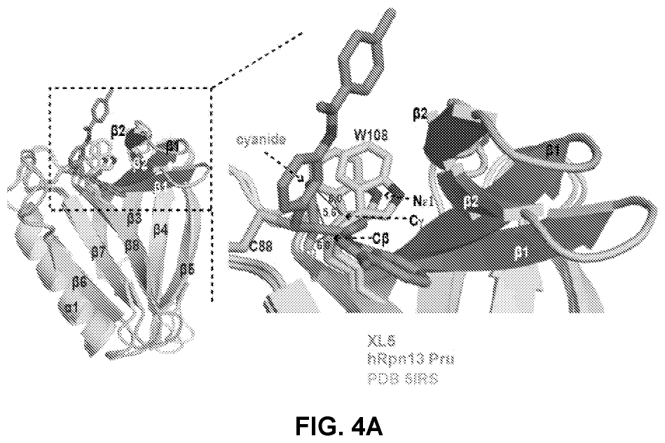

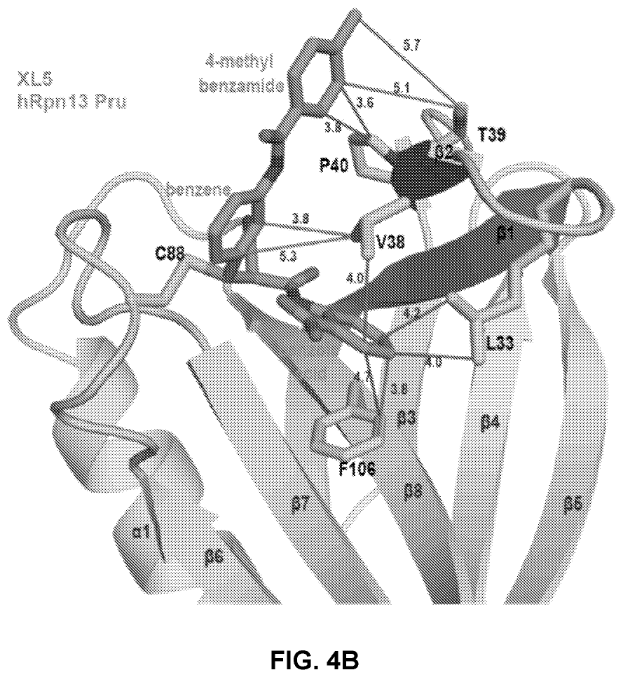

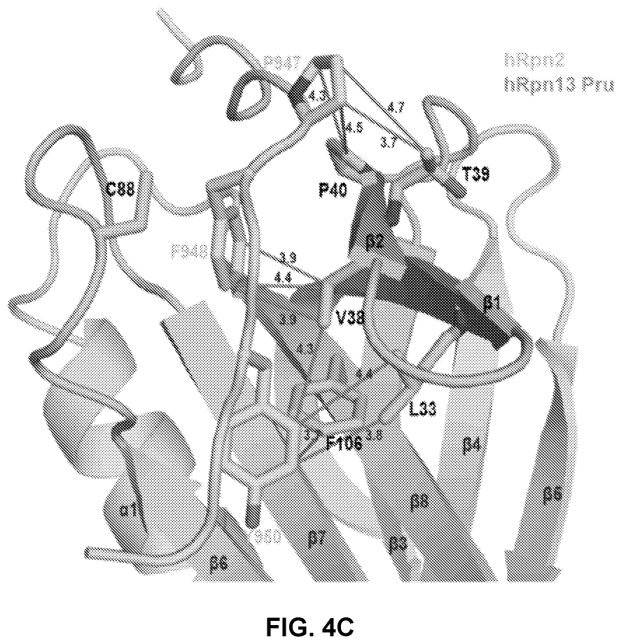

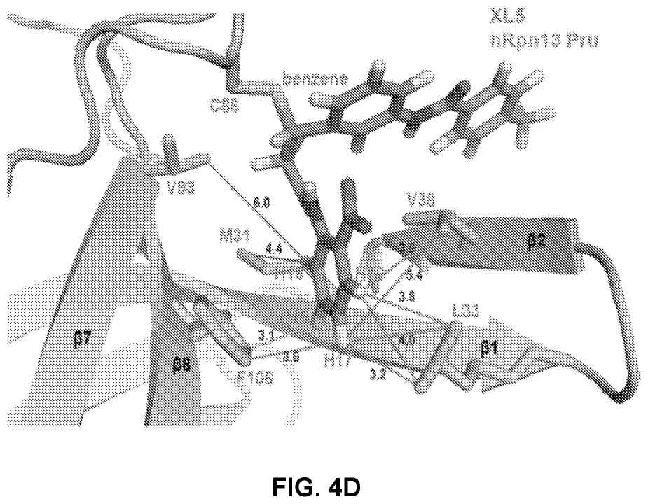

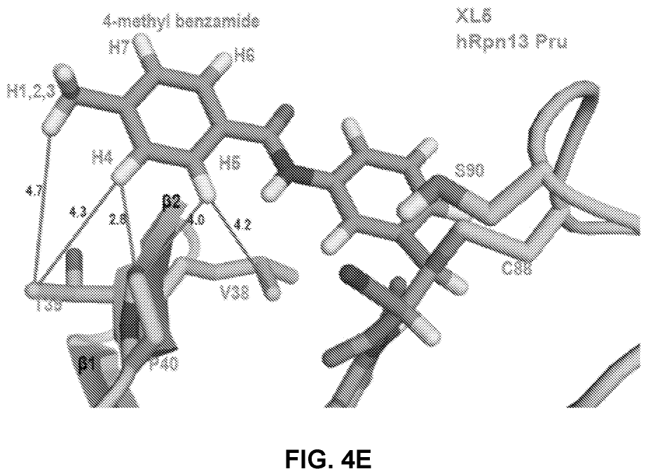

The overall structure of hRpn13 ligated to XL5 is similar to the unligated (PDB 5IRS, FIG. 4A) and hRpn2-bound (PDB 6CO4, FIG. 4C) structures, as expected from the NOEs detected within the structural core. To accommodate XL5, however, the hRpn13 β1-β2 hairpin is shifted away from β8 (FIGS. 4A-4C), allowing intercalation of the benzoic acid group within a hydrophobic pocket formed by β1 L33, β2 V38, and β8 F106 (FIG. 4B). In the XL5-ligated structure, hRpn13 W108 Hβ and Cγ are close to XL5 H19 and the cyanide group (FIG. 4A). These interactions coupled with the change in chemical environment of W108 due to the reconfiguration of local structure (FIG. 4A) provides an explanation for its observed Hε1 and amide resonance shifting (FIG. 1B) and reduction of intrinsic emission at λ₃₅₀ (FIG. 1A).

XL5 binds to hRpn13 Pru with a similar affinity as hRpn2 (944-953) and forms analogous interactions. The central aromatic ring is positioned close to where hRpn2 F948 binds and similarly interacts with V38 while the XL5 4-methyl benzamide binds hRpn13 T39 and P40 similarly compared to hRpn2 P947 (FIGS. 4B-4C). The shorter distance between the central benzene and benzoic acid groups of XL5 relative to hRpn2 F948 and Y950 (which are separated by E949) alters interactions with hRpn13 L33, V38 and F106 causing this end of XL5 to be buried (FIGS. 4B-4C). Consistent with this burying of the benzoic acid aromatic ring (FIG. 4D), inclusion of additional chemical groups to the XL5 scaffold caused reduced affinity. A bulky ortho-trifluoromethyl group (XL30) caused ˜8-fold reduced affinity; this group would form steric clashes with the L33 methyl groups if bound in the same configuration as XL5. Reduced affinity was similarly caused by addition of methoxy (XL28) or methylamino (XL29) groups at the meta position.

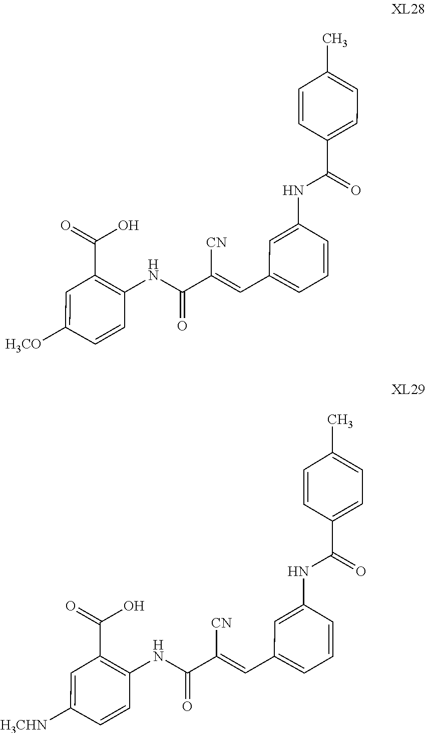

As XL5 H13 and H19 are directed towards the p6-P7 loop, the cyanide group is positioned to form a weak hydrogen bond to the hRpn13 S90 hydroxy group, placing the central benzene ring proximal to V38 and P89 (FIG. 3E). Addition of a trifluoromethyl group (XL32) or methylamino group (XL33) at either ortho position of the XL5 central benzene ring reduced binding affinity to hRpn13 and the structure suggests that this reduction is due to steric clashes with V38. NMR signals of the central XL5 benzene ring are absent, which may stem from anion-π interactions formed between the XL5 carboxylic acid group and central benzene (FIG. 4D); a similar broadening mechanism is reported for an anion (fluoride)-π (thiophene) interactions system. Replacement of this ortho carboxyl group with sulfonamide (XL31) strongly reduced affinity for hRpn13, potentially due to weakening of the XL5 anion-π interaction (FIG. 4D). This part of the structure is well-defined (FIG. 3D) by NOE interactions observed to each end of XL5 as well as to H13 and H19 (FIGS. 3A-3B).

XL5 4-methyl benzamide interacts with the C-terminal end of hRpn13 P2 through hydrophobic interactions (FIGS. 4D-4E), which are indicated in the NOESY data (FIGS. 3A-3B). Modification of the 4-methyl benzamide ring to less hydrophobic 6-hydroxy-5-methyl-pyridine (XL27) reduced affinity compared to XL5 (FIG. 4E), demonstrating the importance of these interactions. The XL5 4-methyl benzamide aromatic ring interacts with the P2 V38 methyl group that is close to the central benzene and P40. The methyl group interacts favorably with that of hRpn13 T39 (FIG. 4E) and its removal in XL23, coupled with inclusion of an ortho-chlorine, reduces affinity by >2-fold and substitution with trifluoromethyl (XL26) or carboxymethyl amino (XL25) groups similarly reduced affinity for hRpn13 Pru. Substitution of the methyl group however with a methylamino group (XL24) had little effect.

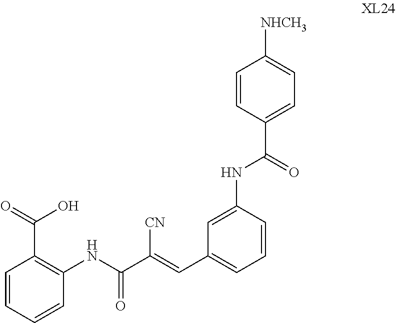 [SMILES: CNc1ccc(C(=O)Nc2cccc(/C=C(\C#N)C(=O)Nc3ccccc3C(=O)O)c2)cc1]

### Example 7: Expansion of XL5 to Incorporate PROTAC Labeling

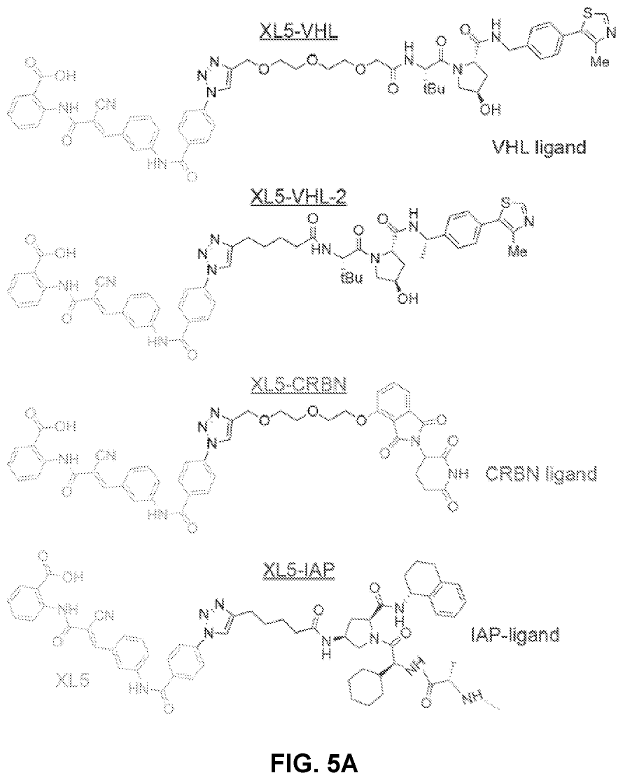

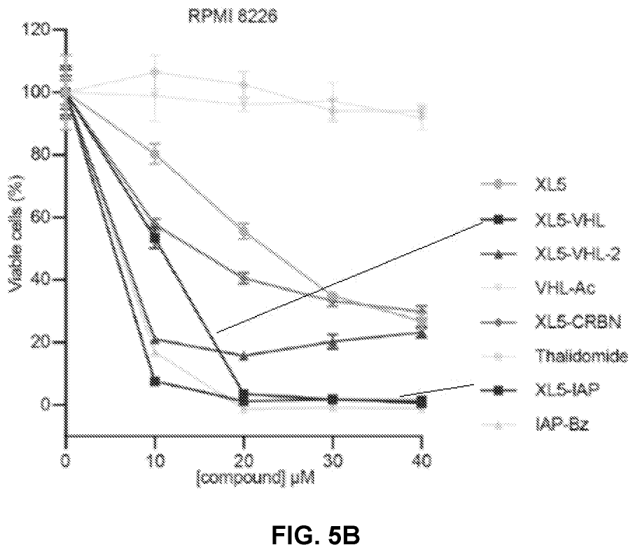

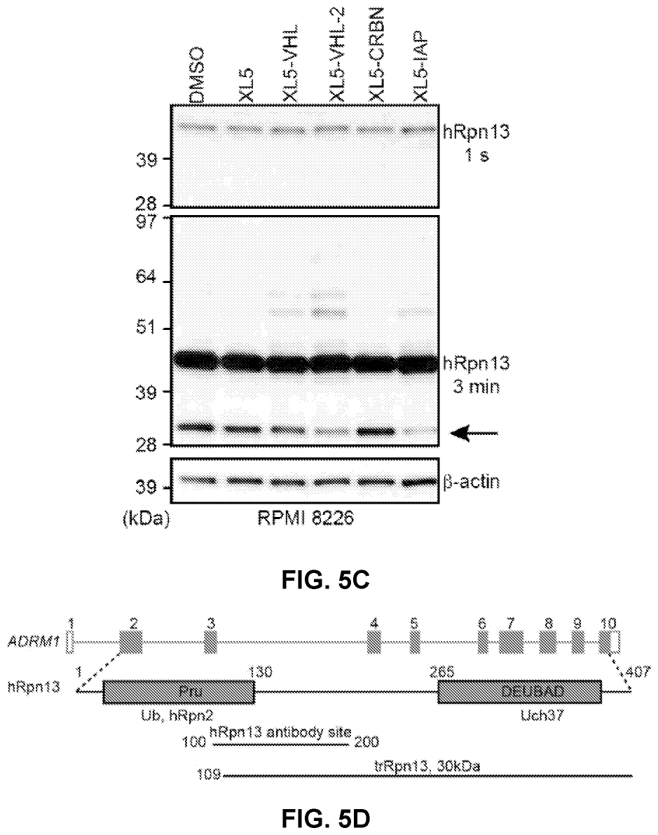

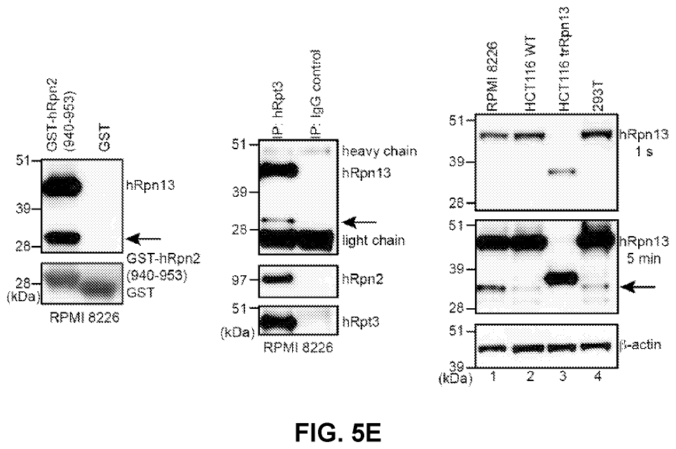

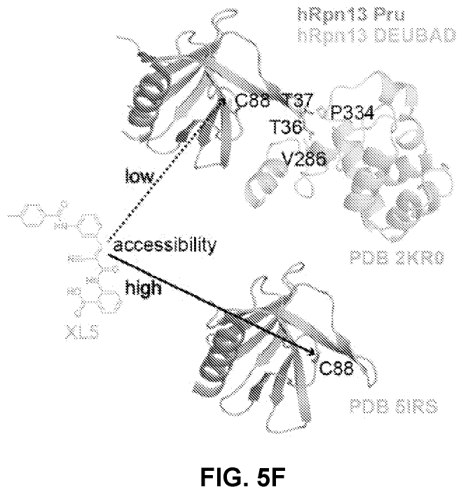

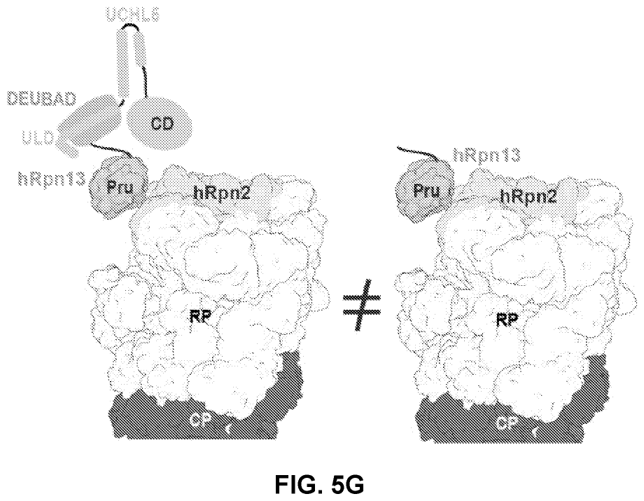

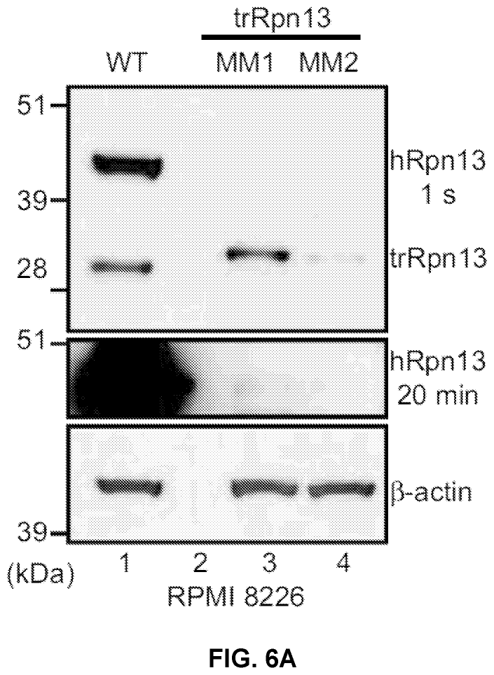

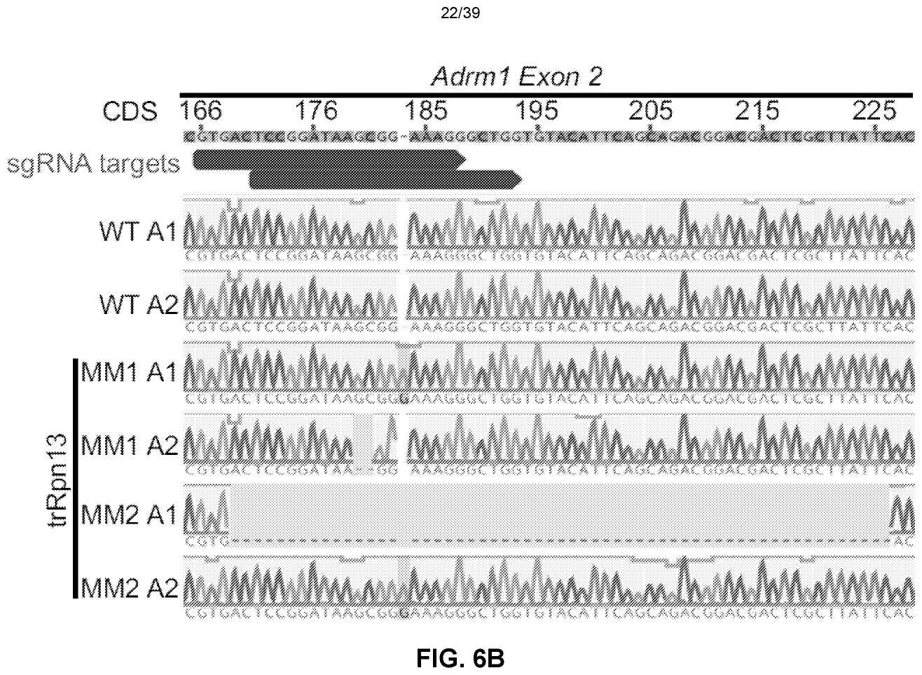

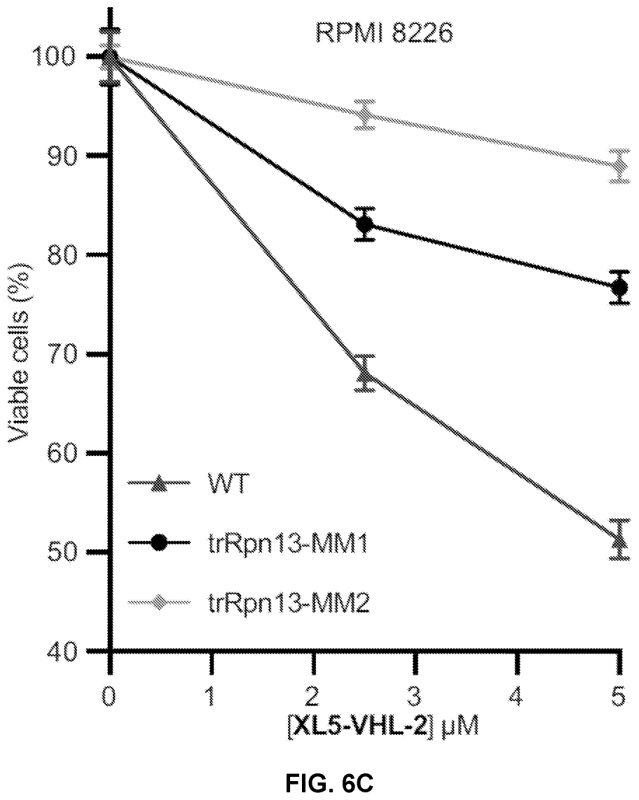

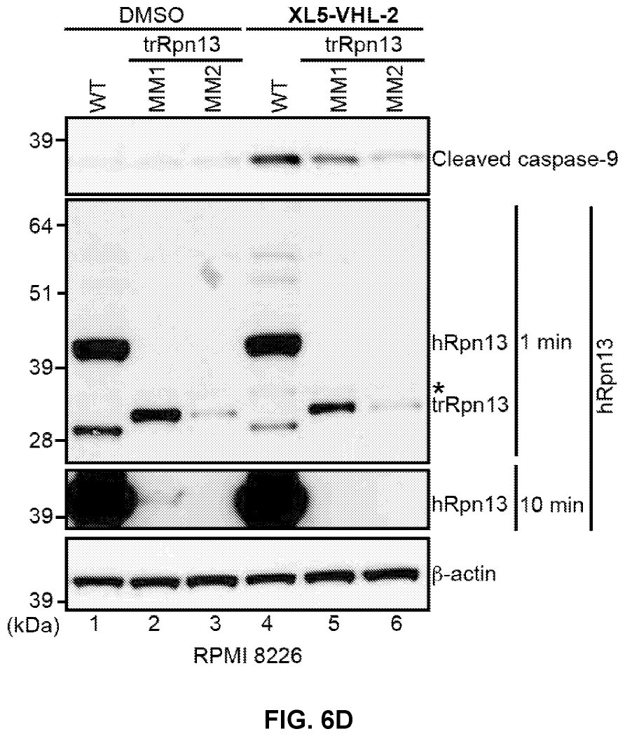

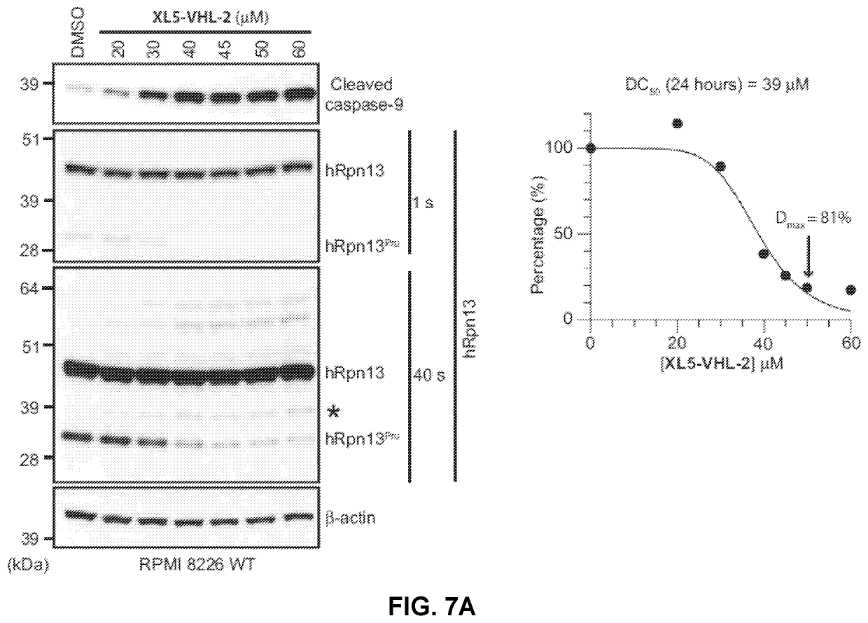

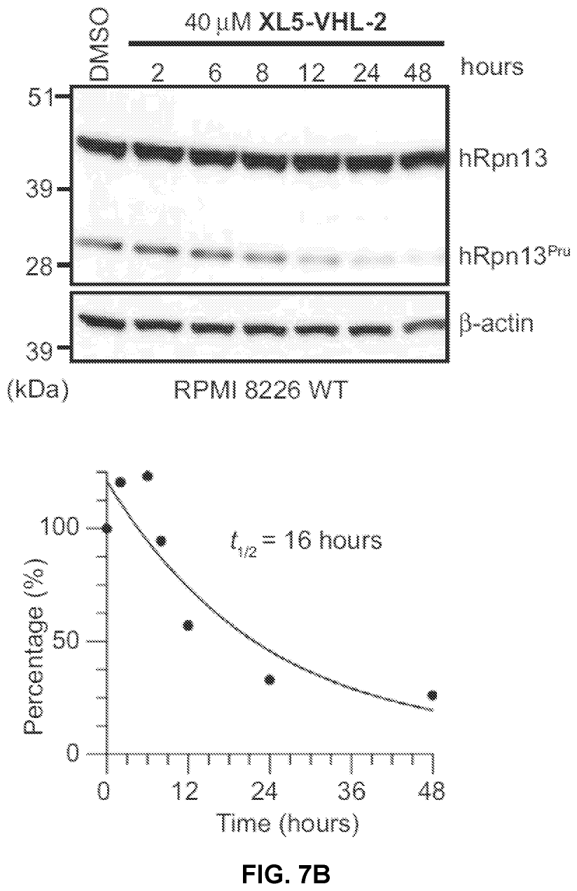

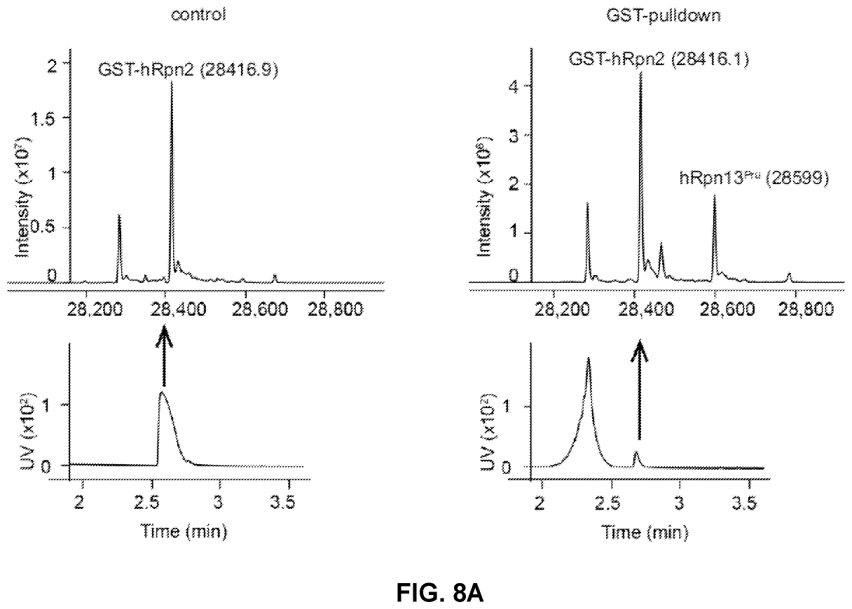

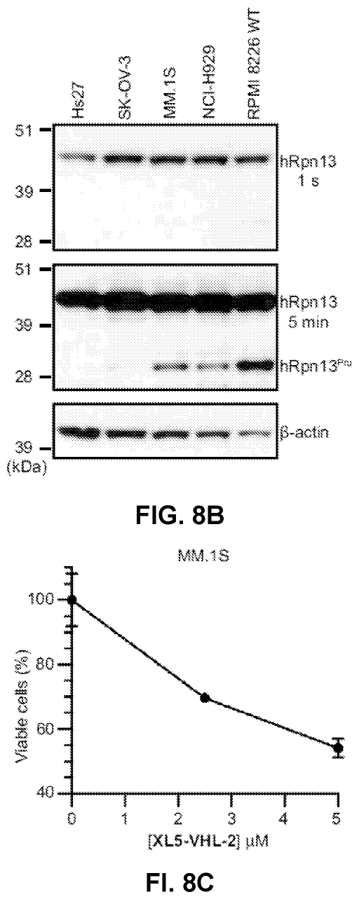

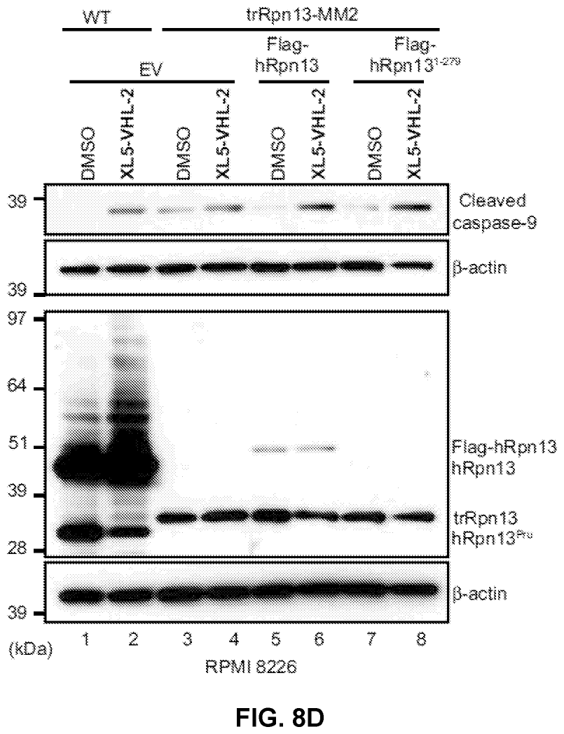

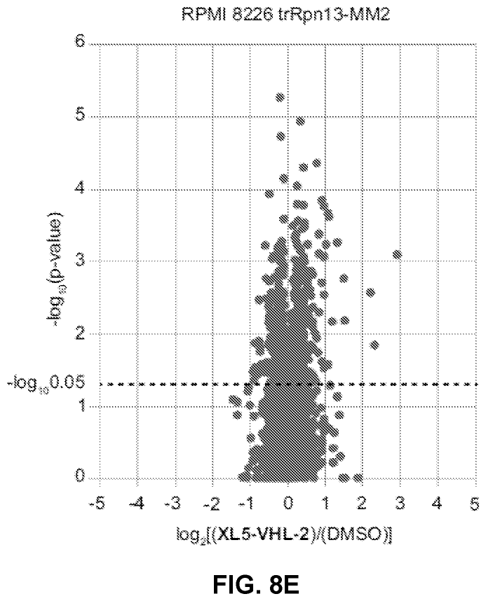

Based on the structure and chemical probing described above, we extended XL5 at the methyl group position to include either of three established PROTACs, namely Von-Hippel Lindau (VHL, with two different linkers to XL5 and in one case VHL modification), cereblon (CRBN) or inhibitor of apoptosis (IAP) (FIG. 5A). An MTT assay demonstrated greater cellular sensitivity when XL5 was fused to a PROTAC (FIG. 5B) with the hook effect4 observed for cells treated with XL5-VHL-2. Control reagents VHL-Ac (for VHL) and thalidomide (for cereblon) did not affect metabolic activity even at 40 μM treatment; however, RPMI 8226 cells were sensitive to IAP ligand (IAP-Bz, FIGS. 5A-5B), which is reported to induce apoptosis.

To test whether the XL5-PROTACs cause ubiquitination and degradation of hRpn13, lysates from RPMI 8226 (FIG. 5C) cells treated with 40 μM XL5 orXL5-PROTAC compared to DMSO vehicle control were immunoprobed for hRpn13 and β-actin (loading control). The level of hRpn13 was similar in all treated RPMI 8226 cells (FIG. 5C); however, following longer exposure of the membrane an increase in higher molecular weight hRpn13 species characteristic of ubiquitination was observed for cells treated with XL5-VHL, XL5-VHL-2 or XL5-IAP (FIG. 5C). In addition, a lower molecular weight species was found in the hRpn13 immunoblot that was reduced in abundance by treatment with XL5-VHL, XL5-VHL-2 or XL5-IAP (FIG. 5C).

The hRpn13 antibody epitope spans amino acids 100-200 (Abcam, personal communication) which includes the hRpn13 Pru (FIG. 5D). To investigate further whether the observed hRpn13 species contains an intact Pru, we tested whether it binds GST-hRpn2 (940-953), which encompasses the hRpn13-binding site at the proteasome and immunoprecipitates endogenous hRpn13 from cells33. GST (as a control) or purified GST-hRpn2 (940-953) was incubated with whole cell lysates from RPMI 8226 cells and mixed with glutathione Sepharose 4B resin. After washing, resin-bound proteins were separated by SDS-PAGE and immunoblotted for hRpn13 or GST. Full-length hRpn13 and the lower molecular weight species were both pulled-down by GST-hRpn2 (940-953) and not by the GST control (FIG. 5E, left panel). We next tested whether this truncated hRpn13 species is present at the proteasome of RPMI 8226 cells by immunoprecipitating whole cell lysates with anti-hRpt3 or rabbit IgG (as a control) antibodies and probing for hRpn13 as well as Rpn2 and Rpt3, as controls. Full length hRpn13, as expected, and the truncated hRpn13 species were detected at the proteasome (FIG. 5E, middle panel).

We reasoned that if the dominant mechanism of action in hRpn13-dependent apoptosis of cancer cells is against the smaller hRpn13 product then it should be present at reduced levels in HCT116 cells compared to RPMI 8226 cells, as XL5 demonstrated greater efficacy in the multiple myeloma cells (FIG. 2C). We therefore immunoprobed lysates from RPMI 8226, HCT116, and the non-cancer 293T cell line for hRpn13 with comparison to β-actin as a loading control. Full length hRpn13 was observed in all three cell lines (FIG. 5E, right panel), as expected. The truncated hRpn13 species was readily observed in RPMI 8226 cells and at markedly reduced levels in HCT116 and 293T cells (FIG. 5E, right panel, lane 1, 2 and 4). We also tested for the presence of this species in the trRpn13 HCT116 cell line, which as described above has a Pru domain-encoding exon deleted, to find it absent as expected (FIG. 5E, right panel, lane 1 versus lane 3). The trRpn13 cells produce an hRpn13 protein product that spans M109 to D407 with molecular weight of −30 kDa (FIG. 5D), slightly larger than the truncated hRpn13 product observed in RPMI 8226 cells (FIG. 5E, right panel).

### Example 8: Discussion and Conclusions

Here, we use a protein-protein interaction surface as a target for small molecule binding, taking advantage of a peripheral cysteine residue for covalent ligation. The strategy of using noncatalytic cysteine residues for small molecule targeting offers advantages for drug discovery and chemical biology. Cysteine-targeting cyanoacrylamide electrophiles form reversible covalent bonds and have been used to inhibit protein kinases with prolonged on-target residence time and higher selectivity and reversible covalent PROTACs have been developed to degrade protein kinases with higher selectivity than noncovalent or irreversibly covalent PROTACs. Although XL5 derivatives XL23-XL33 with modifications of 4-methyl benzamide (R1, R2, R3, X), benzoic acid (R4, R5, R6) or central benzene (R7, R8) groups bind to hRpn13 with similar or weaker binding affinity than XL5, modification of H4, H8, H9, H10, H14, H15, H16 as well as different chemical groups in R1-R8 or X may be exploited to improve affinity. Beyond the region targeted by XL5, the binding cleft continues where hRpn2 prolines P945, P944, and P942 form myriad interactions. We expect that XL5 could be extended to higher affinity by mimicking these interactions, perhaps by addition to the 4-methyl benzamide or H8 (FIG. 3A and FIG. 4B-4C). In summary, we provide here a compound and scaffold for targeting hRpn13 that can be further optimized for higher affinity and preclinical development.

Our data suggest that a truncated hRpn13 species is expressed with an intact hRpn13 Pru and no UCHL5-binding DEUBAD (FIG. 5D) and that this hRpn13 species, which is missing the intramolecular interaction between the Pru and DEUBAD domains, is preferentially targeted by XL5 (FIG. 5F). The truncated hRpn13 species was missed in our previous studies with 293T and HCT116 cell lines, most likely due to the low expression of this product in these cells (FIG. 5E, right panel, 1s versus 5 min exposure for hRpn13). The higher expression level in RPMI 8226 cells coupled with the invocation of XL5-PROTACs enabled us to discover this protein product. A remaining question however is why this truncated hRpn13 product is upregulated in multiple myeloma cells, how pervasive and frequent it is in cancer cells, and whether indeed it is the loss of this protein product that leads to cancer cell death. Premature termination codons formed in mRNA are reported in tumors, as is exon skipping to alter protein-protein interactions. No evidence of hRpn13 exon skipping was observed in HCT116 cells, although future long-read mRNA sequencing may be needed in RPMI 8226 cells to fully rule out this possibility. It is also possible that the hRpn13 mRNA is modified to suppress production of the full length protein. This truncated hRpn13 species harboring the intact Pru but lacking the DEUBAD would be an effective competitor for binding to ubiquitinated substrates and the proteasome, as these intermolecular interactions require displacement of the hRpn13 interdomain interactions. Moreover, hRpn13 activity for this protein product would be uncoupled from the UCHL5 deubiquitinase (FIG. 5G), which hydrolyzes branched ubiquitin chains. Collectively, these effects could change the turnover of proteasome substrates and drive dysregulated cellular proliferation.

Altogether, our studies have provided new reagents for targeting hRpn13 that uncovered the presence of an hRpn13 species upregulated in the multiple myeloma cell line particularly sensitive to these compounds. Specific knockdown of this hRpn13 product and not the full length protein by gene editing or RNAi methods is not feasible; however, the XL5-PROTAC compounds specifically target the more exposed binding surface of the truncated hRpn13 product. This approach is expected to yield a more specific therapeutic effect, with less potential for toxicity by targeting only the upregulated truncated protein.

### Example 9: Synthesis and Characterization of Compounds

### General Information

Starting materials were used as received unless otherwise noted. All moisture sensitive reactions were performed in an inert atmosphere of argon with oven dried glassware. Reagent grade solvents were used for extractions and flash chromatography. Reaction progress was monitored by LC-MS analysis performed on an Agilent UPLC/MS instrument equipped with a RP-C18 column (Poroshell 120 SB-C18, 4.6×50 mm, 2.7 μm or Zorbax 300SB-C18, 4.6×50 mm, 3.5 μm), dual atmospheric pressure chemical ionization (APCI)/electrospray (ESI) mass spectrometry detector, and photodiode array detector. Flash chromatography was performed by using a RediSepRf NP-silica (40-63 μm 60 Å) or a Teledyne RediSepRf Gold RP-C18 column (20-40 μm 100 Å) in a Teledyne ISCO CombiFlash Rf 200 purification system unless otherwise specified. ¹H NMR spectra were recorded on an Agilent 400 MHz or Bruker 800 MHz spectrometer and are reported in parts per million (ppm) on the δ scale relative to CDCl₃ (δ 7.26) and DMSO-d₆ (δ 2.50) as internal standards. Data are reported as follows: chemical shift, multiplicity (s=singlet, d=doublet, t=triplet, q=quartet, b=broad, m=multiplet), coupling constants (Hz), and integration. ¹³C-NMR spectra were recorded on an Agilent 100 MHz or Bruker 200 MHz spectrometer and are reported in parts per million (ppm) on the δ scale relative to CDCl₃ (δ 77.00) and DMSO-d₆ (δ 39.52). Note: The recorded ¹H NMRs of ¹³C₆ labeled compounds are very complex and difficult to interpret due to large couplings between proton and ¹³C-carbon. To remove the large couplings of H-¹³C, BilevelDec ¹H NMR method was used for all ¹³C₆ labeling compounds and reported both the ¹H NMR and BilevelDec ¹H NMR data.

### Synthesis and Characterization Data

XL5-¹³C₆-CB, XL25, XL26, XL27, XL30, XL31, XL32, XL33, XL5-VHL, XL5-VHL-2, XL5-CRBN, XL5-IAP, VHL-Ac and IAP-Bz were synthesized according to the procedures described below and characterization data (¹H NMR, ¹³C NMR, ¹⁹F NMR and high-resolution mass spectrometry (HRMS)) are included.

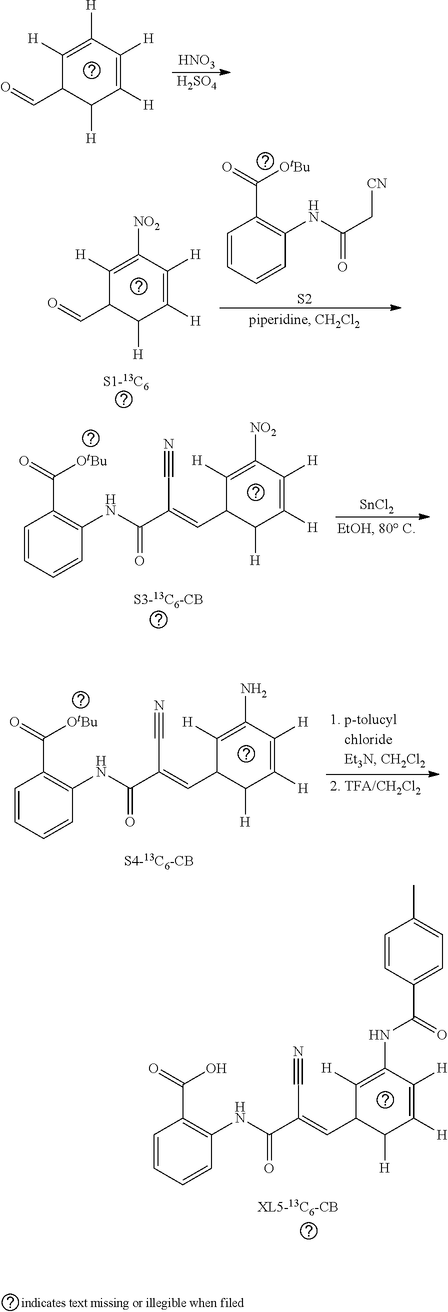

Benz-¹³C₆-aldehyde (500 mg, 4.46 mmol) was placed into a 100 mL round bottom flask and then cold 90% nitric acid (8 mL) was added at −30° C. The resulting yellow solution was warmed to −10° c. and stirred for 30 min. Ice water (20 mL) and EtOAc (30 mL) were then added to the reaction mixture. The separated organic layer was washed with water (2×15 mL), aqueous NaHCO₃ (15 mL), and aqueous NaCl (20 mL). The collected organic layer was dried (Na₂SO₄) and concentrated to provide a crude oil. The crude material was purified by an ISCO combi flash silica gel column (hexanes/EtOAc) to provide 3-nitro-benz-¹³C₆-aldehyde S1-¹³C₆ (430 mg, 61%). Characterization data of S1-¹³C₆: ¹H NMR (400 MHz, CDCl₃): δ 10.10 (dt, J=25.0, 2.1 Hz, 1H), 8.95-8.58 (m, 1H), 8.51-8.37 (m, 1H), 8.30-7.45 (m, 2H); BilevelDec ¹H NMR (400 MHz, CDCl₃) δ 10.13 (d, J=0.8 Hz, 1H), 8.72 (d, J=2.5 Hz, 1H), 8.54-8.41 (m, 1H), 8.23 (dt, J=7.8, 1.3 Hz, 1H), 7.77 (t, J=8.1 Hz, 1H). ¹³C NMR (101 MHz, CDCl₃): δ 189.91 (m), 148.72 (td), 137.38 (td), 134.70 (m), 130.32 (m), 128.46 (ddd), 124.28 (ddd).

In a thick-walled vial, 3-nitro-benz-¹³C₆-aldehyde S1-¹³C6 (200 mg, 1.27 mmol) was dissolved in CH₂Cl₂ (3 mL) and then S2 (331 mg, 1.27 mmol) and piperidine (10 drops) were added at room temperature. The vial was sealed and stirring was continued for 6 h at room temperature. The yellow solid product was collected, washed with CH₂Cl₂ (2×10 mL) and dried under vacuum to afford yellow solid S3-¹³C₆-CB (465 mg, 91%, single E-isomer). ¹H NMR (400 MHz, CDCl₃): δ 12.40 (s, 1H), 9.02-8.52 (m, 3H), 8.44 (t, J=5.1 Hz, 1H), 8.25-8.10 (m, 1H), 8.04 (dd, J=8.0, 1.7 Hz, 1H), 7.99-7.45 (m, 2H), 7.18 (ddd, J=8.2, 7.3, 1.2 Hz, 1H), 1.64 (s, 9H); BilevelDec ¹H NMR (400 MHz, CDCl₃): δ 12.39 (s, 1H), 8.77 (s, 1H), 8.72 (dd, J=8.5, 1.1 Hz, 1H), 8.44 (s, 1H), 8.43-8.34 (m, 2H), 8.04 (dd, J=8.0, 1.7 Hz, 1H), 7.73 (t, J=8.0 Hz, 1H), 7.58 (ddd, J=8.7, 7.3, 1.7 Hz, 1H), 7.18 (ddd, J=8.1, 7.4, 1.2 Hz, 1H), 1.65 (s, 1OH); ¹³C NMR (101 MHz, CDCl₃): δ 167.57, 158.29 (d), 149.84 (m), 148.61 (td), 140.33, 135.26 (dddd), 134.05, 133.38 (td), 131.12, 130.38 (td), 126.59 (m), 125.46 (m), 123.81, 120.94, 117.87, 115.01 (d), 109.51, 83.19, 28.17; HRMS (m/z): [M+Na]+ calcd. for C₁₅¹³C₆H₁₉N₃O₅Na, 422.1424; found, 422.1421 (APCI).

In a thick-walled vial, nitro compound S3-¹³C₆-CB (85 mg, 0.21 mmol) in EtOH (5 mL) was treated with SnCl₂ (202 mg, 1.06 mmol) under argon atmosphere at room temperature. The vial was sealed and heated at 80° C. for 1 hour, after which LC-MS indicated the complete consumption of starting material S3-¹³C₆-CB. The cooled reaction mixture was quenched with aqueous sodium bicarbonate (20 mL) and the product extracted with EtOAc (2×15 mL) and dried (Na₂SO₄). After concentration the crude product was purified by an ISCO combi flash silica gel column (EtOAc/hexanes) to provide yellow colored aniline derivative S4-¹³C₆-CB (62 mg, 80%).

¹H NMR (400 MHz, CDCl₃): δ 12.19 (s, 1H), 8.72 (ddd, J=8.4, 1.2, 0.4 Hz, 1H), 8.28 (t, J=5.7 Hz, 1H), 8.02 (ddd, J=8.0, 1.7, 0.5 Hz, 1H), 7.70-6.51 (m, 6H), 3.78 (bs, 2H), 1.64 (s, 9H); BilevelDec ¹H NMR (400 MHz, CDCl₃): δ 12.19 (s, 1H), 8.72 (dd, J=8.5, 1.2 Hz, 1H), 8.28 (s, 1H), 8.02 (ddd, J=7.9, 1.7, 0.4 Hz, 1H), 7.56 (ddd, J=8.7, 7.3, 1.7 Hz, 1H), 7.38 (d, J=2.0 Hz, 1H), 7.31 (dd, J=20.0, 7.6 Hz, 2H), 7.15 (ddd, J=7.9, 7.3, 1.2 Hz, 1H), 6.86 (d, J=7.7 Hz, 1H), 3.75 (bs, 2H), 1.64 (s, 9H); ¹³C NMR (101 MHz, CDCl₃): δ 167.47, 159.67 (d), 153.61 (d), 146.80 (ddd), 140.63, 133.90, 132.84 (ddd), 131.02, 130.03 (ddd), 123.37, 121.98 (tdd), 120.95, 119.63 (m), 117.84, 116.10, 115.80 (td), 105.42, 82.90, 28.19; HRMS (m/z): [M+H]⁺ calcd. for ¹³C₆C₁₅H₂₂N₃O₃, 370.1862; found, 370.1860.

To a stirred solution of S3-¹³C₆-CB (56 mg, 0.15 mmol) in CH₂Cl₂, p-toluoyl chloride (24 μL, 0.18 mmol) and Et₃N (44 μL, 0.30 mmol) were added at room temperature. Stirring was continued for 12 hours, quenched with water (5 nL), and the product extracted with CH₂Cl₂ (2×6 mL) and dried (Na₂SO₄). The filtrate was concentrated under reduced pressure and purified by an ISCO combi flash silica gel column (EtOAc/hexanes) to afford tert-butyl 2-(2-cyano-3-(3-(4-methylbenzamido)phenyl-¹³C₆)acrylamido)benzoate. The tert-butyl-benzoate product was subjected to 1 mL CH₂Cl₂/TFA (1:1) and stirred for 1 hour at room temperature (monitored by LC-MS). The solvent and TFA were removed under reduced pressure to provide yellow solid material. The solid material was washed with dichloromethane (3×6 mL) to provide pure XL5-¹³C₆-CB (52 mg, 80%). ¹H NMR (400 MHz, DMSO-d₆): δ 13.95 (s, 1H), 12.25 (s, 1H), 10.43 (d, J=3.2 Hz, 1H), 8.70-7.54 (m, 10H), 7.34 (d, J=8.0 Hz, 2H), 7.28-7.20 (m, 1H), 2.38 (s, 3H); BilevelDec ¹H NMR (400 MHz, DMSO-d₆): δ 13.94 (s, 1H), 12.24 (s, 1H), 10.42 (s, 1H), 8.62 (dd, J=8.5, 1.2 Hz, 1H), 8.46 (s, 1H), 8.37 (s, 1H), 8.05 (dd, J=7.9, 1.7 Hz, 1H), 7.94 (d, J=8.0 Hz, 1H), 7.89 (d, J=8.1 Hz, 2H), 7.78 (d, J=7.7 Hz, 1H), 7.67 (ddd, J=8.7, 7.3, 1.7 Hz, 1H), 7.56 (t, J=7.9 Hz, 1H), 7.34 (d, J=8.0 Hz, 2H), 7.25 (td, J=7.6, 1.2 Hz, 1H), 2.38 (s, 3H); ¹³C NMR (101 MHz, DMSO-d₆): δ 170.23, 166.09, 159.45 (d), 153.29 (d), 142.33, 140.65, 140.48 (ddd), 134.74, 132.50 (ddd), 132.15 (d), 131.67, 130.06 (td), 129.41, 128.24, 125.54 (dtd), 124.25, 122.46 (ddd), 120.83, 117.51, 106.53, 21.49; HRMS (m/z): [M+H]⁺ calcd. for ¹³C₆C₁₉H₂₀N₃O₄, 432.1655; found, 432.1660.

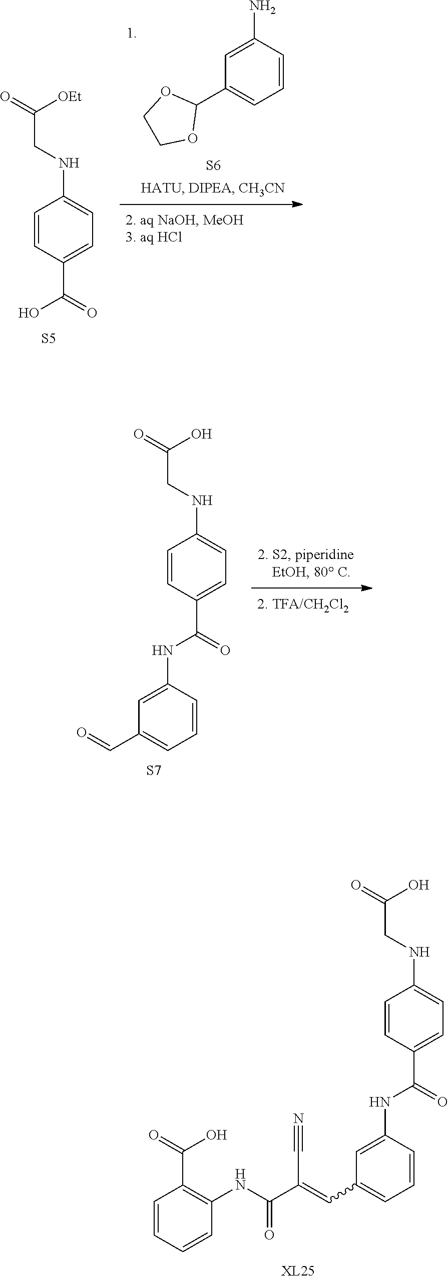

In a 25 mL round bottom flask, 4-((2-Ethoxy-2-oxoethyl)amino)benzoic acid S7 (300 mg, 1.34 mmol) was dissolved in CH₃CN (6 mL) and cooled to 0° C. HATU (612 mg, 1.61 mmol), S6 (254 mg, 1.5 mmol), and DIPEA (0.7 mL, 4 mmol) were added to the cooled solution. The reaction mixture was stirred for 30 min at room temperature at which point LC-MS indicated the complete consumption of S5. The solvent was evaporated, water was added to the reaction mixture and product extracted with EtOAC (2×10 mL) dried over anhydrous Na₂SO₄. After concentration, the crude product was dissolved in MeOH (2 mL) and then aqueous NaOH (2 mL, 1 M) was added at room temperature. The mixture was heated for 3 hours at 50° C. (monitored by LCMS) and cooled to room temperature. Ice cold aqueous HCl (4 mL, 1 M) was added at room temperature and stirred for 1 hour. The reaction mixture extracted with EtOAC (4×10 mL) and the combined organic layers were washed with aqueous NaCl (20 mL) and dried over anhydrous Na₂SO₄. After concentration, the crude product was purified by an ISCO combi flash silica gel column (CH₂Cl₂/MeOH) to afford aldehyde S7 (150 mg, 37%). ¹H NMR (400 MHz, DMSO-d₆): δ 10.09 (s, 1H), 9.99 (s, 1H), 8.36 (t, J=1.9 Hz, 1H), 8.06 (ddd, J=7.9, 2.3, 1.4 Hz, 1H), 7.81 (d, J=8.9 Hz, 2H), 7.68-7.43 (m, 2H), 6.65 (d, J=8.8 Hz, 1H), 3.91 (s, 2H); ¹³C NMR (101 MHz, DMSO-d₆): δ 193.61 (d), 172.61, 165.96, 151.89, 141.05, 137.09, 129.88, 129.81, 129.74, 126.33, 125.10, 121.73, 120.45, 120.43, 111.62, 111.55, 44.66; HRMS (m/z): [M+H]⁺ calcd. for C₁₆H₁₅N₂O₄, 299.1032; found, 299.1033.

In a thick-walled vial, aldehyde S7 (50 mg, 0.17 mmol) was dissolved in ethanol (2 mL) and then S2 (44 mg, 0.17 mmol) and piperidine (10 drops) were added at room temperature. The vial was sealed and heated at 80° C. for 1 hour. The reaction mixture was cooled to room temperature and the solvent was removed by rotary evaporator. The crude material was purified by an ISCO combi flash silica gel column (CH₂Cl₂/MeOH) to afford tert-butyl benzoate (E/Z=6:1). The benzoate was dissolved in 2 mL CH₂Cl₂/TFA (1:1) and stirred for 1 hour. The product was precipitated which was washed with cold CH₂Cl₂ (10 mL) to provide the pure XL25 (48 mg, 59%, E/Z=6:1). Characterization data of major isomer: ¹H NMR (400 MHz, DMSO-d₆): δ 12.23 (s, 1H), 10.10 (s, 1H), 8.63 (dd, J=8.5, 1.2 Hz, 1H), 8.45 (t, J=2.0 Hz, 1H), 8.41-8.32 (m, 1H), 8.06 (dd, J=7.9, 1.7 Hz, 1H), 7.92 (ddd, J=8.1, 2.0, 0.9 Hz, 1H), 7.84-7.77 (m, 2H), 7.77-7.74 (m, 1H), 7.69 (ddd, J=8.7, 7.4, 1.7 Hz, 1H), 7.55 (t, J=8.0 Hz, 1H), 7.27 (td, J=7.6, 1.2 Hz, 1H), 6.70-6.59 (m, 2H), 3.90 (s, 2H); ¹³C NMR (101 MHz, DMSO-d₆): δ 172.61, 170.24, 165.97, 159.53, 153.47, 151.88, 141.01, 140.66, 134.80, 132.43, 131.70, 129.98, 129.79, 125.32, 125.13, 124.28, 122.44, 121.76, 120.87, 117.48, 116.04, 111.58, 106.37, 44.67; HRMS (m/z): [M+H]⁺ calcd. for C₂₆H₂₁N₄O₆, 485.1461; found, 485.1463.

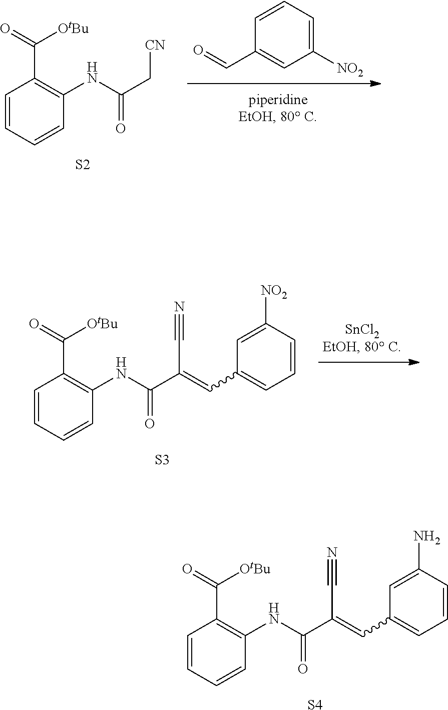

In a thick-walled vial, 2-cyano-N-arylacetamide S2 (1 g, 3.84 mmol) was dissolved in ethanol (10 mL) and then 3-nitrobenzaldehyde (577 mg, 3.84 mmol) and piperidine (10 drops) were added at room temperature. The vial was sealed and heated at 80° C. for 1 hour. The reaction mixture was cooled to room temperature and the precipitate was collected, washed with ethanol (2×10 mL) and dried under vacuum to afford yellow solid S3 (1.33 g, 89%, single E-isomer). ¹H NMR (400 MHz, CDCl₃): δ 12.41 (s, 1H), 8.80-8.76 (m, 1H), 8.75-8.70 (m, 1H), 8.45 (s, 1H), 8.43-8.34 (m, 2H), 8.05 (dd, J=8.0, 1.7 Hz, 1H), 7.78-7.70 (m, 1H), 7.65-7.52 (m, 1H), 7.19 (dddd, J=8.0, 7.3, 1.2, 0.7 Hz, 1H), 1.65 (d, J=0.7 Hz, 9H); ¹³C NMR (101 MHz, CDCl₃): δ 167.57, 158.30, 150.14, 148.64, 140.33, 135.24, 134.06, 133.46, 131.14, 130.43, 126.60, 125.47, 123.82, 120.95, 117.88, 115.03, 109.51, 83.20, 28.18; HRMS (m/z): [M+Na]⁺ calcd. for C₂₁H₁₉N₃O₅Na, 416.1222; found, 416.1222 (APCI).

Nitrobenzene S3 (800 mg, 2 mmol) in ethanol (15 mL) was placed into a thick-walled vial and SnCl₂ (1.89 g, 10 mmol) was added under argon atmosphere at room temperature. The sealed vial was heated at 80° C. for 2 hours, after which LCMS indicated the complete consumption of starting material S3. The cooled reaction mixture was quenched with aqueous sodium bicarbonate (20 mL) and the product extracted with EtOAc (3×10 mL) and dried (Na₂SO₄). After concentration, the crude product was purified by an ISCO combi flash silica gel column (EtOAc/hexanes) to provide aniline derivative S4 (545 mg, 75%). ¹H NMR (400 MHz, CDCl₃): δ 12.22 (s, 1H), 8.74 (d, J=8.4 Hz, 1H), 8.30 (d, J=2.3 Hz, 1H), 8.03 (d, J=8.0 Hz, 1H), 7.58 (t, J=8.0 Hz, 1H), 7.40 (s, 1H), 7.37-7.26 (m, 3H), 7.16 (t, J=7.7 Hz, 1H), 6.87 (d, J=7.8 Hz, 1H), 3.88 (s, 2H), 1.65 (d, J=2.2 Hz, 9H); ¹³C NMR (101 MHz, CDCl₃): δ 167.49, 159.71, 153.69, 147.03, 140.64, 133.94, 132.84, 131.07, 130.07, 123.39, 121.98, 120.96, 119.60, 117.82, 116.14, 115.74, 105.35, 82.93, 28.20; HRMS (m/z): [M+Na]⁺ calcd. for C₂₁H₂₁N₃O₃Na, 386.1481; found, 386.1482.

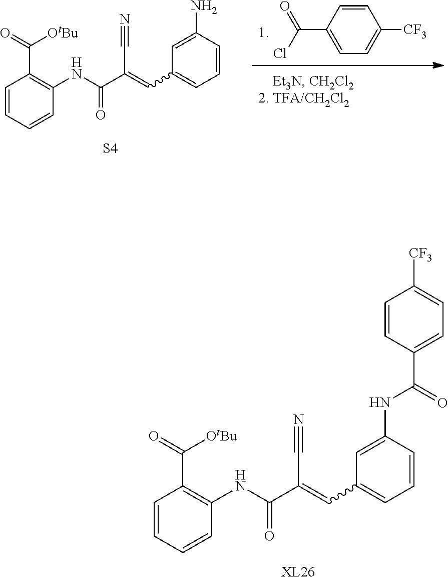

XL26: In a 20 mL round bottom flask, compound S4 (50 mg, 0.14 mmol) was dissolved in CH₂Cl₂ (3 mL). 4-(Trifluoromethyl)benzoyl chloride (32 mg, 0.15 mmol) and Et₃N (30 μL, 0.21 mmol) were added at 0° C. Solvent was evaporated and purified by an ISCO combi flash silica gel column (EtOAc/hexanes). The product was dissolved in 2 mL CH₂Cl₂/TFA (1:1) and stirred for 3 hours at room temperature. After the deprotection was completed, the solvent was removed and purified by an ISCO combi flash silica gel column (CH₂Cl₂/MeOH) to provide XL26 (30 mg, 45% over two steps). ¹H NMR (400 MHz, DMSO-d₆): δ 12.31 (s, 1H), 10.75 (s, 1H), 8.62 (dd, J=8.5, 1.1 Hz, 1H), 8.47 (t, J=1.9 Hz, 1H), 8.39 (s, 1H), 8.19-8.11 (m, 2H), 8.05 (dd, J=7.9, 1.7 Hz, 1H), 7.94 (ddt, J=10.3, 7.6, 0.9 Hz, 3H), 7.81 (ddd, J=8.3, 1.7, 0.8 Hz, 1H), 7.67 (ddd, J=8.6, 7.4, 1.7 Hz, 1H), 7.60 (t, J=8.0 Hz, 1H), 7.25 (td, J=7.6, 1.2 Hz, 1H); ¹³C NMR (101 MHz, DMSO-d₆): δ 170.24, 165.18, 159.43, 153.13, 140.63, 140.07, 138.88, 134.69, 132.63, 131.68, 130.20, 129.17, 126.54, 125.94 (q), 125.34, 124.26, 122.43, 120.82, 117.70, 115.96, 106.76; HRMS (m/z): [M+H]⁺ calcd. for C₂₅H₁₇N₃O₄F₃, 480.1171; found, 480.1173.

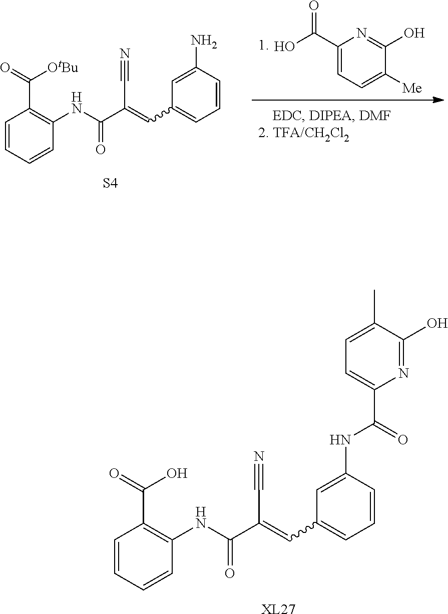

In a 10 mL round bottom flask, 6-hydroxy-5-methylpicolinic acid (52 mg, 0.34 mmol) was dissolved in DMF (2 mL). EDC-HCl salt (107 mg, 0.56 mmol), S4 (100 mg, 0.28 mmol), and DIPEA (0.21 mL) were added at room temperature. The reaction mixture was stirred overnight and DMF was removed by rotary evaporator. The crude material was purified by an ISCO combi flash silica gel column (CH₂Cl₂/MeOH). Fractions were combined and the solvent was removed to provide tert-butyl benzoate derivative. tert-Butyl benzoate was subjected to 2 mL CH₂Cl₂/TFA (1:1) to deprotect the tert-butyl group. After 1 hour, dichloromethane and TFA were removed by rotary evaporator to yield yellow solid. The solid was washed with CH₂Cl₂ (5 mL) to afford XL27 (8 mg, 6% over two steps). ¹H NMR (800 MHz, DMSO-d₆): δ 11.46 (s, 1H), 10.55 (s, 1H), 8.54 (d, J=8.2 Hz, 1H), 8.35 (d, J=2.4 Hz, 1H), 8.27 (s, 1H), 8.08 (dd, J=7.7, 1.8 Hz, 1H), 7.94 (dd, J=8.2, 2.1 Hz, 1H), 7.80 (d, J=7.7 Hz, 1H), 7.60 (q, J=7.9, 6.6 Hz, 2H), 7.44 (t, J=7.8 Hz, 1H), 7.26 (s, 2H), 7.11 (t, J=7.5 Hz, 1H), 2.14 (s, 3H); ¹³C NMR (201 MHz, DMSO-d₆) δ 169.80, 161.55, 158.90, 150.63, 140.30, 138.91, 132.35, 131.07, 129.67, 128.86, 127.68, 125.26, 123.81, 122.57, 121.60, 119.49, 119.08, 117.99, 116.49, 115.57, 115.00, 108.07, 16.18; HRMS (m/z): [M+H]⁺ calcd. for C₂₄H₁₉N₄O₅, 443.1355; found, 443.1350.

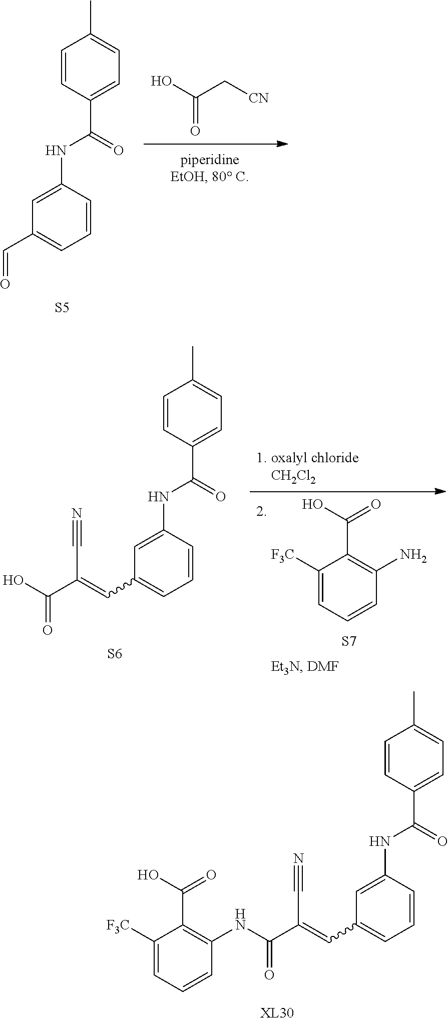

In a thick-walled vial, aldehyde S5 (1 g, 4.18 mmol) was dissolved in ethanol and then cyanoacetic acid (355 mg, 4.18 mmol), and piperidine (10 drops) were added at room temperature. The vial was sealed and heated at 80° C. for 1 hour. The reaction mixture was cooled to room temperature and the precipitate was collected, washed with ethanol (2×10 mL) and dried under vacuum to yield product S6 (1.1 g, 86%, single E-isomer). ¹H NMR (400 MHz, DMSO-d₆): δ 10.43 (s, 1H), 8.44 (t, J=1.9 Hz, 1H), 8.28 (s, 1H), 7.94 (ddd, J=8.2, 2.2, 1.0 Hz, 1H), 7.91-7.85 (m, 2H), 7.78-7.72 (m, 1H), 7.56 (t, J=8.0 Hz, 1H), 7.41-7.28 (m, 2H), 2.39 (s, 3H); ¹³C NMR (101 MHz, DMSO-d₆): δ 166.08, 163.66, 154.75, 142.32, 140.44, 132.36, 132.14, 130.02, 129.42, 128.28, 126.15, 125.47, 122.59, 116.23, 104.73, 21.52; HRMS (m/z): [M+H]⁺ calcd. for C₁₈H₁₅N₂O₃, 307.1083; found, 307.1083.

In a 25 mL round bottom flask, the above compound S6 (100 mg, 0.33 mmol) was dissolved in CH₂Cl₂ (6 mL) and cooled to 0° C. Oxalyl chloride (1 mL) and DMF (1 drop) were added to the cooled solution. The reaction mixture was warmed to room temperature and stirred for 2 hours (monitored by LC-MS). The solvent and excess oxalyl chloride were removed by rotary evaporator to provide the corresponding acid chloride of S6. To a stirred solution of acid chloride in CH₂Cl₂ (5 mL), S7 (75 mg, 0.36 mmol) and Et₃N (184 μLt, 1.32 mmol) were added at 0° C. The reaction mixture was warmed to room temperature and stirred for 6 hours. The solvent was removed and purified by an RP-C18 ISCO combi flash column (Water and MeCN, water was buffered with 0.05% TFA) to yield XL30 (60 mg, 37%). ¹H NMR (400 MHz, DMSO-d₆): δ 10.43 (s, 1H), 10.37 (s, 1H), 8.47 (t, J=1.9 Hz, 1H), 8.28 (s, 1H), 8.01-7.84 (m, 4H), 7.83-7.69 (m, 3H), 7.58 (t, J=8.0 Hz, 1H), 7.44-7.26 (m, 2H), 2.38 (s, 3H); ¹³C NMR (101 MHz, DMSO-d₆) δ 167.03, 166.12, 161.30, 142.36, 140.53, 135.70, 132.49, 132.15, 131.40, 131.03, 130.11, 129.43, 128.24, 127.42, 127.11, 125.38, 125.20, 125.05, 124.64, 124.59, 122.47, 116.15, 106.74, 21.48; HRMS (m/z): [M+H]⁺ calcd. for C₂₆H₁₉N₃O₄F₃, 494.1328; found, 494.1326.

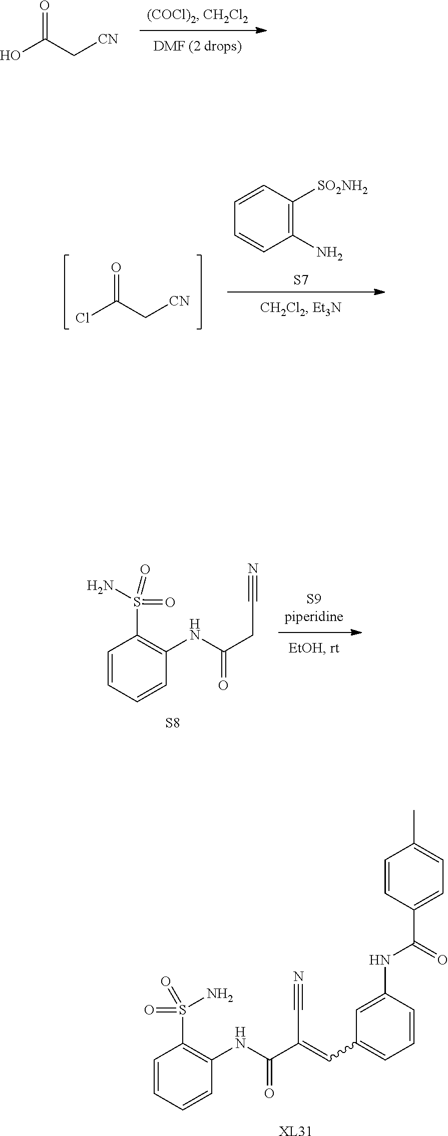

In a 25 mL round bottom flask cyanoacetic acid (1 g, 1175 mmol) was dissolved in CH₂Cl₂ (10 mL) and cooled to 0° C. Oxalyl chloride (1.3 mL, 15.27 mmol) and DMF (2 drops) were added at 0° C. The reaction mixture was warmed to room temperature and stirred for 1 hour. Solvent and excess oxalyl chloride were removed to provide 2-cyanoacetyl chloride. Cyanoacetyl chloride was dissolved in CH₂Cl₂ (10 mL) and cooled to 0° C. 2-Aminobenzenesulfonamide S7 (2.02 g, 11.75 mmol) and Et₃N (2.2 mL, 15.26 mmol) were added to the cooled solutions. The reaction mixture was warmed to room temperature and stirred overnight. Water (5 mL) was added to the reaction mixture and extracted with CH₂Cl₂ (2×15 mL). The combined organic layers were dried with anhydrous Na₂SO₄ and purified by a silica gel ISCO combi flash column (CH₂Cl₂/MeOH) to afford S8 (2.1 g, 75%). ¹H NMR (400 MHz, DMSO-d₆): δ 9.46 (s, 1H), 7.87 (d, J=8.0 Hz, 2H), 7.73-7.53 (m, 3H), 7.37 (t, J=7.8 Hz, 1H), 4.06 (s, 2H); ¹³C NMR (101 MHz, DMSO-d₆) δ 162.44, 135.17, 134.29, 133.27, 128.17, 126.14, 116.23, 27.46; HRMS (m/z): [M+Na]⁺ calcd. for C₉H₉N₃O₃SNa, 262.0262; found, 262.0262.

In a thick-walled vial, S8 (100 mg, 0.42 mmol) was dissolved in CH₂Cl₂ (3 mL) and then S5 (100 mg, 0.42 mmol) and piperidine (10 drops) were added at room temperature. The vial was sealed and stirring was continued for 2 hours at room temperature. The solid product was collected, washed with CH₂Cl₂ (2×10 mL) and dried under vacuum to provide XL31 (155 mg, 80%, single E-isomer). ¹H NMR (400 MHz, DMSO-d₆): δ 10.46 (s, 1H), 10.15 (s, 1H), 8.49 (t, J=2.0 Hz, 1H), 8.36 (s, 1H), 8.20 (dd, J=8.3, 1.2 Hz, 1H), 7.97 (dd, J=8.3, 2.1 Hz, 1H), 7.94-7.86 (m, 3H), 7.82-7.79 (m, 1H), 7.76 (s, 2H), 7.68 (ddd, J=8.6, 7.5, 1.6 Hz, 1H), 7.60 (t, J=8.0 Hz, 1H), 7.41 (dd, J=7.7, 1.2 Hz, 1H), 7.37 (d, J=7.9 Hz, 2H), 2.41 (s, 3H); ¹³C NMR (101 MHz, DMSO-d₆) δ 166.12, 159.90, 152.52, 142.36, 140.54, 134.64, 133.60, 133.44, 132.55, 132.16, 130.14, 129.47, 129.42, 128.30, 125.73, 125.51, 125.19, 124.34, 122.50, 116.11, 107.06, 21.48. ¹⁹F NMR (376 MHz, DMSO-d₆) 5-57.94; HRMS (m/z): [M+H]⁺ calcd. for C₂₄H₂₁N₄O₄S, 461.1284; found, 461.1282.

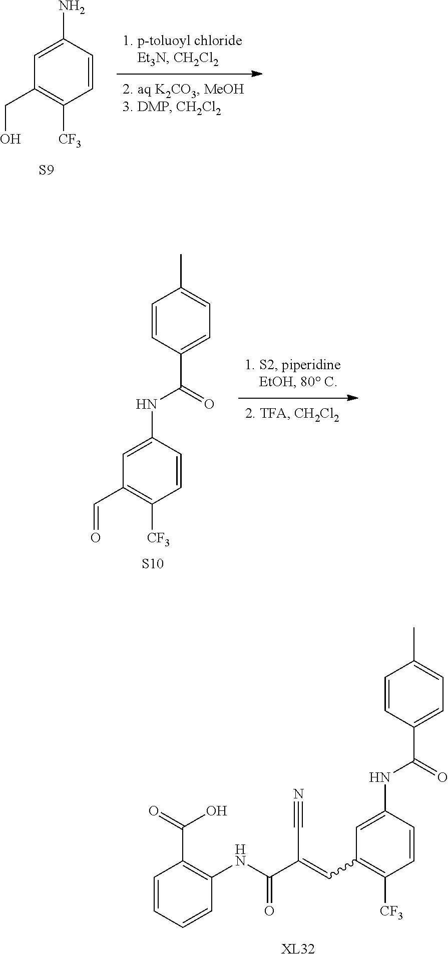 [SMILES: Nc1ccc(C(F)(F)F)c(CO)c1 ; "S9" ; Cc1ccc(C(=O)Nc2ccc(C(F)(F)F)c(C=O)c2)cc1 ; "S10" ; Cc1ccc(C(=O)Nc2ccc(C(F)(F)F)c(C=C(C#N)C(=O)Nc3ccccc3C(=O)O)c2)cc1 ; "XL32"]

In a 25 mL round bottom flask, 5-amino-2-(trifluoromethyl)benzyl alcohol S9 (250 mg, 1.31 mmol) was dissolved in CH₂Cl₂ (5 mL) and cooled to 0° C. p-Toluoyl chloride (506 mg, 3.27 mmol) and Et₃N (0.8 mL, 5.24 mmol) were added to the cooled solution. The reaction mixture was warmed to room temperature and stirring continued overnight. Water (5 mL) was added to the reaction mixture and the product extracted with CH₂Cl₂ (10 mL×2). The combined organic layers were dried over anhydrous Na₂SO₄, filtered, and concentrated to give the crude ditoluoylated product. The crude material was dissolved in MeOH (5 mL) and then K2CO₃ (903 mg, 6.55 mmol) was added at room temperature. The reaction mixture was stirred for 30 min and filtered through a pad of celite. The solvent was removed to provide the benzyl alcohol. The crude benzyl alcohol in CH₂Cl₂ (10 mL) was cooled to 0° C. and DMP (848 mg, 2 mmol) added. The reaction mixture was warmed to room temperature and stirred for 1 hour. The reaction mixture was quenched with aqueous Na₂S₂O₃ (5 mL) and product extracted with CH₂Cl₂ (2×10 mL). The combined organic layers were dried over anhydrous Na₂SO₄, filtered, concentrated and purified by an ISCO Combi flash column (SiO2, hexanes/EtOAc) to yield aldehyde S10 (215 mg, 53% over 3 steps). ¹H NMR (400 MHz, CDCl₃): δ 10.39 (d, J=2.1 Hz, 1H), 8.56-8.38 (m, 1H), 8.33 (s, 1H), 8.09 (d, J=2.4 Hz, 1H), 7.81 (d, J=8.0 Hz, 3H), 7.32 (d, J=7.7 Hz, 2H), 2.45 (s, 3H); ¹³C NMR (101 MHz, CDCl₃): δ 188.74, 165.96, 143.30, 141.93, 134.41, 130.99, 129.66, 127.24, 126.26, 125.06, 124.12, 119.47, 119.41, 21.59; ¹⁹F NMR (376 MHz, CDCl₃): δ −54.86; HRMS (m/z): [M+H]⁺ calcd. for C₁₆H₁₃NO₂F₃, 308.0898; found, 308.0899.

In a thick-walled vial, S10 (200 mg, 0.65 mmol) was dissolved in ethanol (5 mL) and then S2 (170 mg, 0.65 mmol) and piperidine (10 drops) were added at room temperature. The vial was sealed and heated at 80° C. for 1 hour. The reaction mixture was cooled to room temperature and the solvent was evaporated. The crude material was purified by an ISCO combi flash silica gel column (CH₂Cl₂/MeOH to yield the tert-butyl benzoate. The tert-butyl ester (100 mg, 0.18 mmol) was treated with 2 mL CH₂Cl₂/TFA (1:1) to provide the crude product. The product was purified by a silica gel ISCO combi flash column (CH₂Cl₂/MeOH) to yield XL32 (65 mg, 73%, single E-isomer). ¹H NMR (400 MHz, DMSO-d₆): δ 12.35 (s, 1H), 10.76 (s, 1H), 8.67-8.60 (m, 2H), 8.57 (d, J=2.1 Hz, 1H), 8.18 (dd, J=8.7, 2.1 Hz, 1H), 8.07 (dd, J=7.9, 1.7 Hz, 1H), 7.96-7.89 (m, 3H), 7.69 (ddd, J=8.7, 7.4, 1.7 Hz, 1H), 7.37 (d, J=7.9 Hz, 2H), 7.28 (td, J=7.7, 1.2 Hz, 1H), 2.40 (s, 3H); ¹³C NMR (101 MHz, DMSO-d₆): δ 170.28, 166.50, 158.28, 150.00, 143.84, 142.79, 140.40, 134.81, 131.72, 131.69, 131.50, 129.46, 128.45, 128.06, 125.66, 124.56, 122.95, 122.48-121.82 (m), 120.88, 120.81, 117.56, 114.63, 112.55, 21.54; ¹⁹F NMR (376 MHz, DMSO-d₆): δ −57.32; HRMS (m/z): [M+H]⁺ calcd. for C₂₆H₁₉N₃O₄F₃, 494.1328; found, 494.1327.

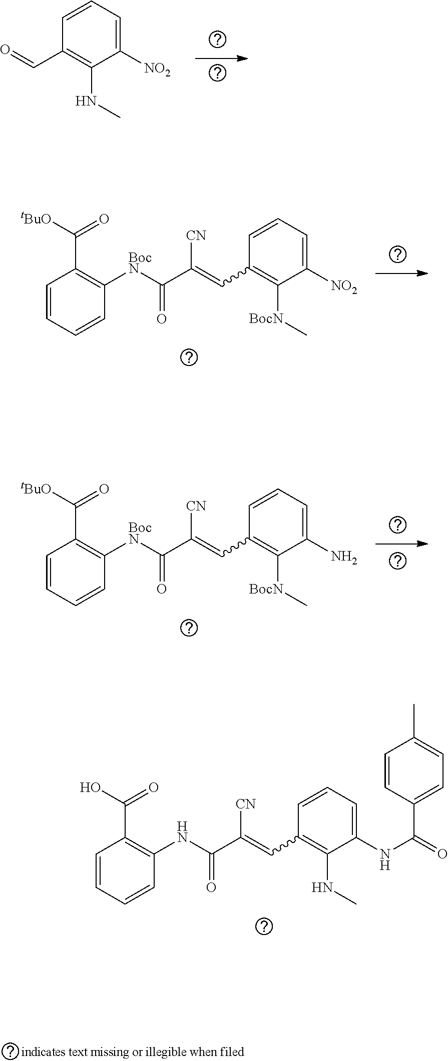 [SMILES: CNc1c(C=O)cccc1[N+](=O)[O-] ; CN(C(=O)OC(C)(C)C)c1c(C=C(C#N)C(=O)N(C(=O)OC(C)(C)C)c2ccccc2C(=O)OC(C)(C)C)cccc1[N+](=O)[O-] ; CN(C(=O)OC(C)(C)C)c1c(N)cccc1C=C(C#N)C(=O)N(C(=O)OC(C)(C)C)c1ccccc1C(=O)OC(C)(C)C ; CNc1c(C=C(C#N)C(=O)Nc2ccccc2C(=O)O)cccc1NC(=O)c1ccc(C)cc1]

In a thick-walled vial, 2-(methylamino)-3-nitrobenzaldehyde (158 mg, 0.87 mmol), was dissolved in CH₂Cl₂ (3 mL) and then S2 (228 mg, 0.87 mmol), and piperidine (10 drops) were added at room temperature. The vial was sealed and stirring was continued for 6 hours at room temperature. The solid product was collected, washed with CH₂Cl₂ (2×10 mL) to yield the tert-butyl benzoate. The tert-butyl benzoate (100 mg, 0.24 mmol) was dissolved in CH₂Cl₂ (5 mL) and then di-tert-butyl dicarbonate (400 mg, 1.83 mmol), Et₃N (264 μLt, 1.83 mmol) and catalytic amounts of 4-dimethylaminopyridine were added at room temperature. The reaction mixture was stirred for 12 hours. Water (5 mL) was added to the reaction mixture and the product extracted with CH₂Cl₂ (2×6 mL) and dried (Na₂SO₄). After concentration, the crude product was purified by an ISCO combi flash silica gel column (EtOAc/hexanes) to yield S11 (143 mg, 96%). Nitrobenzene S11 (100 mg, 0.16 mmol) in ethanol (15 mL) was placed into a thick-walled vial and SnCl₂ (152 mg, 0.80 mmol) was added under an argon atmosphere at room temperature. The sealed vial was heated at 80° C. for 1 hour, after which LC-MS indicated the complete consumption of starting material S11. The cooled reaction mixture was quenched with aqueous sodium bicarbonate (20 mL), product extracted with EtOAc (3×15 mL) and dried (Na₂SO₄). After concentration, the crude product was purified by an ISCO combi flash silica gel column (EtOAc/hexanes) to provide aniline derivative S12 (73 mg, 77%, single E-isomer). ¹H NMR (400 MHz, CDCl₃): δ 8.00 (ddd, J=7.8, 1.7, 0.4 Hz, 1H), 7.84 (s, 1H), 7.70 (ddd, J=7.9, 1.3, 0.4 Hz, 1H), 7.57 (ddd, J=7.9, 7.4, 1.6 Hz, 1H), 7.41 (ddd, J=7.8, 7.4, 1.3 Hz, 1H), 7.04 (t, J=7.7 Hz, 1H), 6.99-6.92 (m, 1H), 6.85 (dd, J=7.7, 1.5 Hz, 1H), 3.73 (s, 3H), 1.56 (s, 18H), 1.23 (s, 9H); ¹³C NMR (101 MHz, CDCl₃): δ 167.74, 164.47, 159.29, 156.29, 151.64, 138.60, 137.42, 137.06, 132.57, 132.09, 130.91, 130.64, 130.56, 130.09, 128.07, 124.90, 123.95, 120.18, 119.70, 82.98, 81.34, 78.31, 42.22, 28.55, 28.21, 27.68; HRMS (m/z): [M+H]⁺ calcd. for C₃₂H₄₁N₄O₇, 593.2975; found, 593.2980.

XL33 was synthesized from S12 using the same procedure as XL5-¹³C₆-CB from S4-¹³C₆-CB. Characterization data of XL33: ¹H NMR (400 MHz, DMSO-d₆): δ 11.96 (s, 1H), 10.84 (s, 1H), 9.50 (d, J=15.6 Hz, 2H), 9.04 (s, 1H), 8.31 (d, J=8.2 Hz, 1H), 8.05 (dd, J=7.9, 1.6 Hz, 1H), 8.00 (dd, J=7.8, 1.5 Hz, 1H), 7.95 (d, J=8.1 Hz, 2H), 7.88 (dd, J=7.6, 1.5 Hz, 1H), 7.73-7.65 (m, 2H), 7.40 (d, J=8.0 Hz, 2H), 7.33 (t, J=7.7 Hz, 1H), 3.73 (s, 3H), 2.41 (s, 3H); ¹³C NMR (101 MHz, CDCl₃): δ 169.66, 165.87, 163.26, 155.14, 143.29, 143.04, 139.38, 135.92, 134.82, 134.29, 131.59, 130.74, 129.69, 129.34, 128.42, 128.14, 126.83, 125.15, 123.51, 122.51, 120.29, 119.37, 41.12, 21.53; HRMS (m/z): [M+H]⁺ calcd. for C₂₆H₂₃N₄O₄, 455.1719; found, 455.1727.

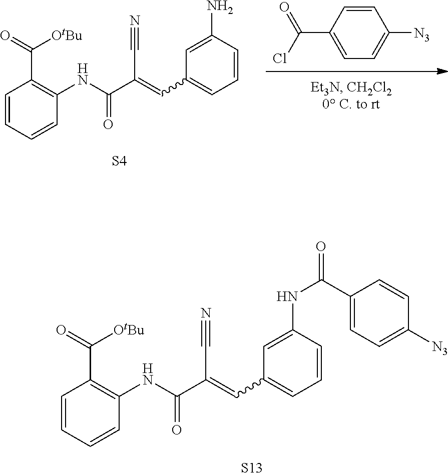

In a 25 mL round bottom flask, S4 (300 mg, 0.82 mmol) was dissolved in CH₂Cl₂ (6 mL) and cooled to 0° C. The freshly prepared 4-azidobenzoyl chloride (167 mg, 0.91 mmol) and triethylamine (0.24 mL, 1.64 mmol) were added to the cooled solution. The reaction mixture was warmed to room temperature and stirred for 5 hours. Water (5 mL) was added to the reaction mixture and product extracted with CH₂Cl₂ (2×10 mL). The combined layers were dried over anhydrous Na₂SO₄, filtered, and concentrated to provide crude product. The crude material was purified by a silica gel ISCO combi flash column (hexanes/EtOAc) to yield S13 (390 mg, 93%). ¹H NMR (400 MHz, CDCl₃): δ 12.16 (s, 1H), 8.84 (s, 1H), 8.68 (dd, J=8.4, 1.1 Hz, 1H), 8.27-8.16 (m, 2H), 7.98 (dd, J=8.0, 1.6 Hz, 1H), 7.94 (ddd, J=8.2, 2.2, 0.9 Hz, 1H), 7.90-7.82 (m, 2H), 7.74-7.61 (m, 1H), 7.50 (ddd, J=8.7, 7.3, 1.7 Hz, 1H), 7.40 (t, J=8.0 Hz, 1H), 7.11 (ddd, J=8.2, 7.4, 1.2 Hz, 1H), 6.97-6.86 (m, 2H), 1.58 (s, 9H); ¹³C NMR (101 MHz, CDCl₃): δ 167.48, 165.34, 159.21, 153.05, 143.71, 140.48, 139.15, 133.93, 132.45, 131.08, 130.84, 129.76, 129.29, 126.67, 124.95, 123.49, 122.20, 120.87, 118.92, 117.71, 115.86, 106.06, 82.94, 28.10; HRMS (m/z): [M+Na]⁺ calcd. for C₂₈H₂₄N₆O₄Na, 531.1757; found, 531.1760.

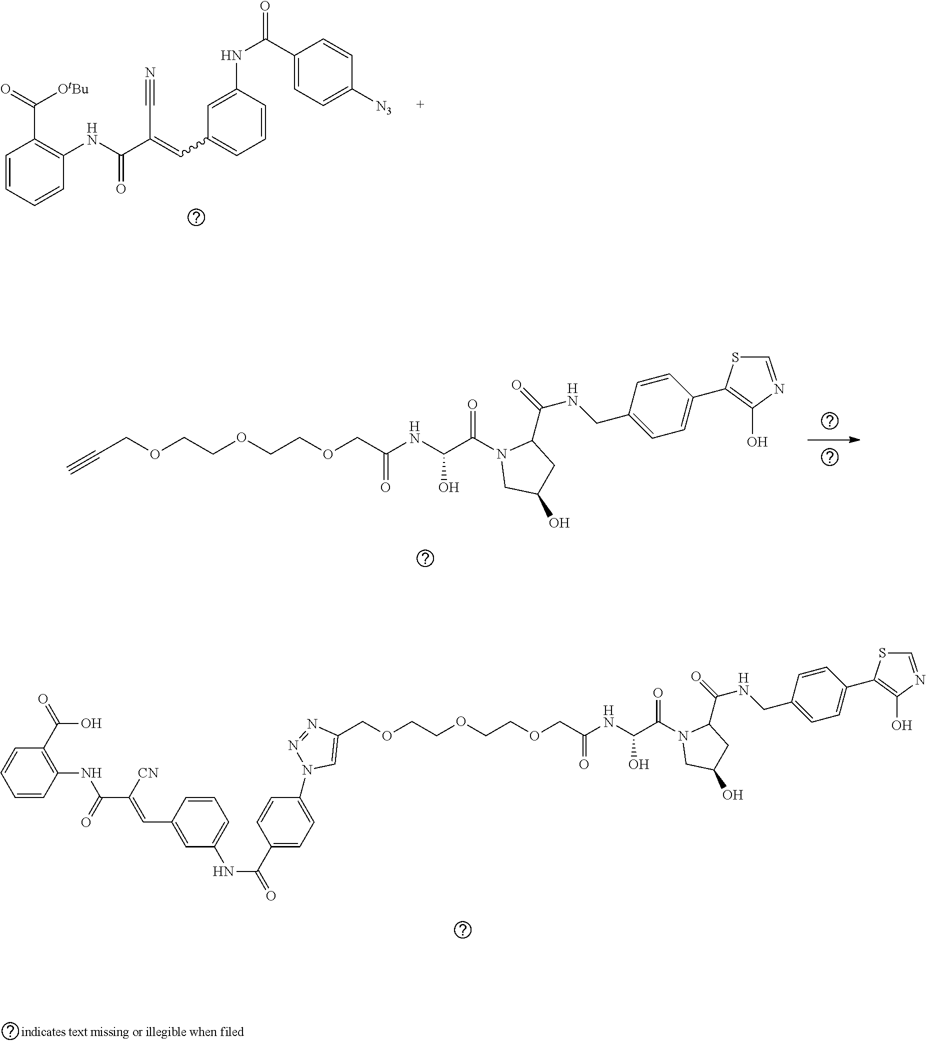 [SMILES: CC(C)(C)OC(=O)c1ccccc1NC(=O)C(C#N)=Cc1cccc(NC(=O)c2ccc(N=[N+]=[N-])cc2)c1 ; C#CCOCCOCCOCC(=O)N[C@@H](O)C(=O)N1C[C@H](O)CC1C(=O)NCc1ccc(-c2scnc2O)cc1 ; N#C/C(=C\c1cccc(NC(=O)c2ccc(-n3cc(COCCOCCOCC(=O)N[C@@H](O)C(=O)N4C[C@H](O)CC4C(=O)NCc4ccc(-c5scnc5O)cc4)nn3)cc2)c1)C(=O)Nc1ccccc1C(=O)O]

Azide S13 (8 mg, 0.016 mmol), alkyne S14 (10 mg, 0.016 mmol) and dry CH₃CN (3 ml) were placed into a round bottom flask equipped with argon, and then CuI (0.3 mg, 0.0016 mmol) and Et₃N (2.3 μL, 0.016 mmol) were added to the reaction mixture. The reaction mixture was stirred overnight at room temperature, then acetonitrile was removed under reduced pressure. The crude material was purified by a silica gel ISCO combi flash column (CH₂Cl₂/MeOH) to yield the click product. The click product was subjected to 1 mL CH₂Cl₂/TFA (1:1) and stirred for 1 hour (monitored by LC-MS). The solvent was removed and purified by a silica gel ISCO combi flash column (CH₂Cl₂/MeOH) to provide XL5-VHL (11 mg, 65% over two-steps). ¹H NMR (400 MHz, DMSO-d₆): δ 10.69 (s, 1H), 8.99 (s, 1H), 8.92 (s, 1H), 8.59 (q, J=7.6, 6.8 Hz, 2H), 8.46 (s, 1H), 8.32 (s, 1H), 8.24-8.16 (m, 2H), 8.16-8.02 (m, 3H), 7.98 (dd, J=7.8, 1.9 Hz, 1H), 7.85-7.77 (m, 1H), 7.60 (t, J=7.9 Hz, 1H), 7.51-7.31 (m, 6H), 7.12 (s, 1H), 5.17 (s, 1H), 4.63 (s, 2H), 4.60-4.52 (m, 1H), 4.50-4.30 (m, 3H), 4.30-4.15 (m, 1H), 3.97 (s, 2H), 3.72-3.51 (m, 1OH), 2.42 (s, 3H), 2.14-2.01 (m, 1H), 1.90 (ddd, J=12.9, 8.8, 4.5 Hz, 1H), 0.93 (s, 9H); ¹³C NMR (101 MHz, DMSO-d₆): δ 172.23, 169.60, 169.10, 167.66, 165.13, 159.47, 151.90, 151.71, 148.18, 145.89, 140.78, 140.19, 139.85, 139.24, 136.67, 134.66, 132.79, 131.67, 130.14, 130.11, 130.05, 129.32, 129.13, 127.91, 125.98, 124.99, 123.31, 122.74, 122.48, 120.06, 120.01, 119.82, 116.12, 108.18, 70.88, 70.20, 70.08, 69.65, 69.31, 63.85, 59.21, 57.01, 56.17, 42.13, 40.39, 38.36, 36.14, 26.61, 16.35; HRMS (m/z): [M+H]⁺ calcd. for C₅₅H₅₉N₁₀O₁₁S, 1067.4085; found, 1067.4063.

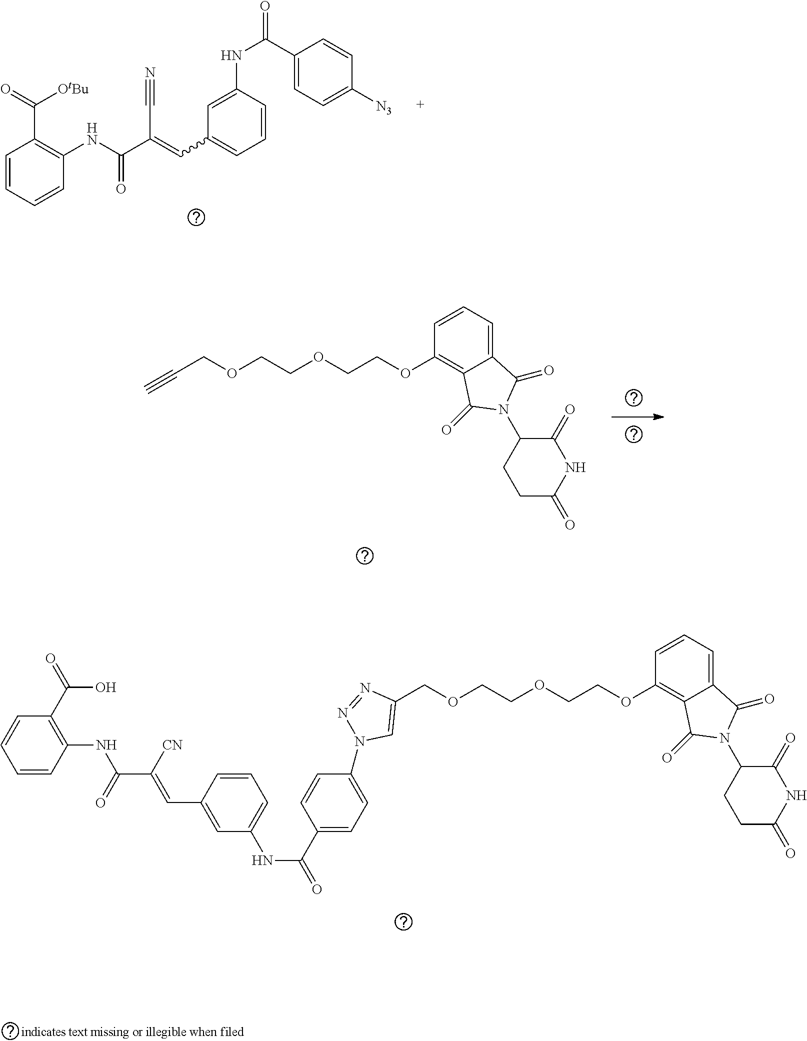 [SMILES: CC(C)(C)OC(=O)c1ccccc1NC(=O)C(C#N)=Cc1cccc(NC(=O)c2ccc(N=[N+]=[N-])cc2)c1 ; C#CCOCCOCCOc1cccc2c1C(=O)N(C1CCC(=O)NC1=O)C2=O ; N#C/C(=C\c1cccc(NC(=O)c2ccc(-n3cc(COCCOCCOc4cccc5c4C(=O)N(C4CCC(=O)NC4=O)C5=O)nn3)cc2)c1)C(=O)Nc1ccccc1C(=O)O]

XL5-CRBN was synthesized using the same reaction sequence as XL5-VHL. Starting materials azide S13 (11 mg, 0.022 mmol), alkyne S14 (10 mg, 0.022 mmol), CuI (0.4 mg, 0.0022 mmol), and Et₃N (3 μL, 0.022 mmol) were used. The crude product was purified by a silica gel ISCO combi flash column (CH₂Cl₂/MeOH) to provide XL5-CRBN (12 mg, 63%, E/Z=5:1). Characterization data of major isomer: ¹H NMR (400 MHz, DMSO-d₆): δ 12.23 (s, 1H), 11.09 (s, 1H), 10.66 (s, 1H), 8.92 (s, 1H), 8.67-8.61 (m, 1H), 8.50 (t, J=2.0 Hz, 1H), 8.41 (s, 1H), 8.19 (d, J=8.7 Hz, 2H), 8.11 (d, J=8.7 Hz, 2H), 8.06 (dd, J=8.0, 1.7 Hz, 1H), 8.02-7.95 (m, 2H), 7.85-7.76 (m, 2H), 7.72-7.66 (m, 1H), 7.61 (t, J=7.9 Hz, 1H), 7.52 (d, J=8.7 Hz, 1H), 7.43 (d, J=7.2 Hz, 1H), 7.32-7.20 (m, 1H), 5.07 (dd, J=12.8, 5.3 Hz, 1H), 4.65 (s, 2H), 4.35 (t, J=4.6 Hz, 2H), 3.82 (t, J=4.6 Hz, 2H), 3.75-3.65 (m, 4H), 2.99-2.77 (m, 1H), 2.68-2.40 (m, 2H), 2.05-1.92 (m, 1H); ¹³C NMR (101 MHz, DMSO-d₆): δ 173.23, 173.13, 170.40, 167.25, 165.74, 165.12, 159.46, 156.27, 153.26, 145.94, 140.64, 140.49, 140.26, 139.26, 137.42, 134.83, 134.65, 133.70, 132.61, 131.72, 130.19, 130.04, 125.42, 124.30, 122.75, 122.49, 120.87, 120.47, 120.06, 117.47, 116.78, 115.99, 115.83, 106.65, 70.57, 69.74, 69.35, 63.90, 55.37, 49.25, 31.40, 22.43; HRMS (m/z): [M+H]⁺ calcd. for C₄₄H₃₇N₈O₁₁, 853.2582; found, 853.2576.

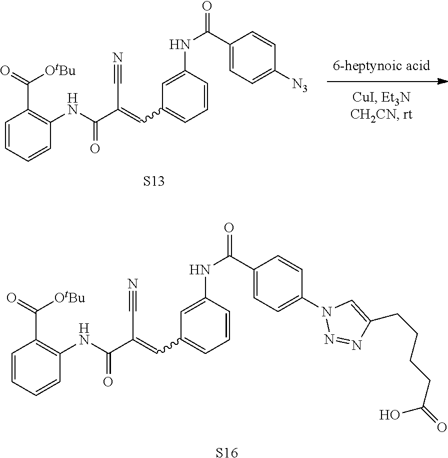 [SMILES: CC(C)(C)OC(=O)c1ccccc1NC(=O)C(C#N)=Cc1cccc(NC(=O)c2ccc(N=[N+]=[N-])cc2)c1 ; "S13" ; CC(C)(C)OC(=O)c1ccccc1NC(=O)C(C#N)=Cc1cccc(NC(=O)c2ccc(-n3cc(CCCCC(=O)O)nn3)cc2)c1 ; "S16"]

Azide S13 (50 mg, 0.098 mmol), 6-heptynoic acid (13 mg, 0.098 mmol) and dry CH₃CN (3 mL) were placed into a round bottom flask equipped with argon, and then CuI (2 mg, 0.0098 mmol) and Et₃N (15 μL, 0.098 mmol) were added to the reaction mixture. The reaction mixture was stirred overnight at room temperature, then acetonitrile was removed under reduced pressure. The crude material was purified by a silica gel ISCO combi flash column (CH₂Cl₂/MeOH) to yield S16 (43 mg, 69%). ¹H NMR (400 MHz, DMSO-d₆): δ 12.01 (s, 1H), 11.58 (s, 1H), 10.64 (s, 1H), 8.71 (d, J=0.7 Hz, 1H), 8.52 (t, J=1.9 Hz, 1H), 8.41-8.31 (m, 2H), 8.24-8.15 (m, 2H), 8.11-8.00 (m, 2H), 8.00-7.89 (m, 2H), 7.79 (ddd, J=8.3, 1.7, 0.8 Hz, 1H), 7.65 (ddd, J=8.4, 7.4, 1.7 Hz, 1H), 7.60 (t, J=8.0 Hz, 1H), 7.27 (ddd, J=7.9, 7.3, 1.2 Hz, 1H), 2.72 (t, J=7.4 Hz, 2H), 2.26 (t, J=7.2 Hz, 2H), 1.76-1.64 (m, 2H), 1.63-1.57 (m, 2H), 1.56 (s, 4H); ¹³C NMR (101 MHz, DMSO-d₆): δ 174.85, 167.14, 165.13, 159.76, 152.99, 148.70, 140.28, 139.46, 139.42, 134.35, 134.27, 132.59, 131.29, 130.17, 130.04, 126.28, 125.28, 124.69, 122.34, 122.06, 120.69, 120.03, 119.74, 116.04, 106.76, 82.94, 33.85, 28.65, 28.17, 25.19, 24.52; HRMS (m/z): [M+H]⁺ calcd. for C₃₅H₃₅N₆O₆, 635.2618; found, 635.2628.

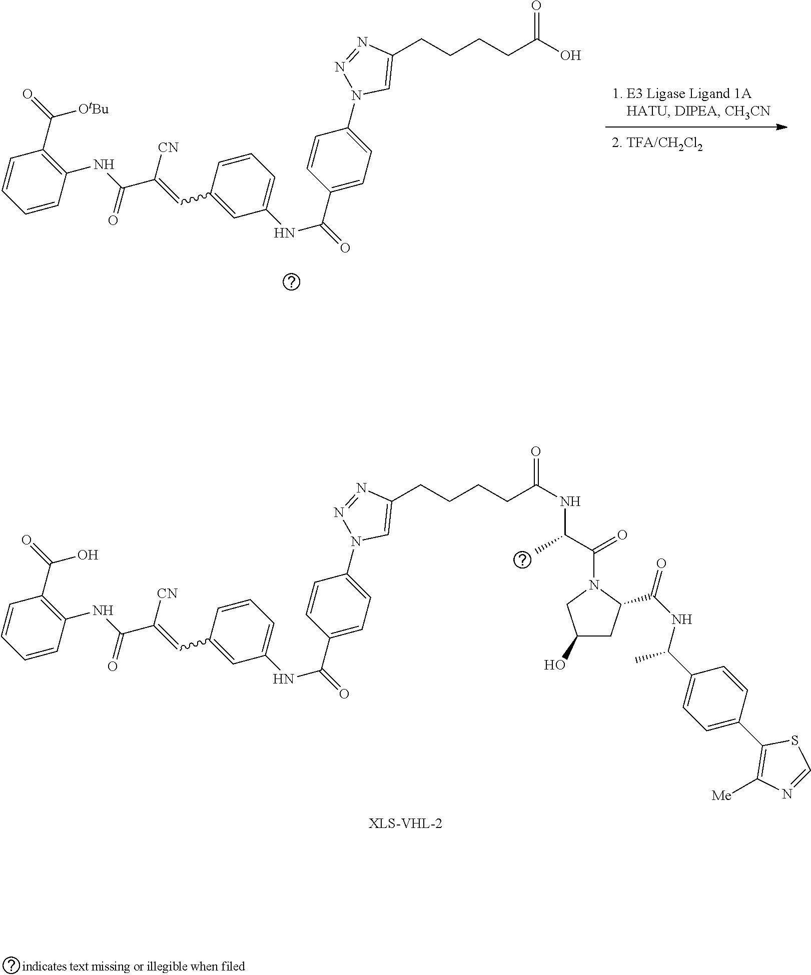 [SMILES: CC(C)(C)OC(=O)c1ccccc1NC(=O)C(C#N)=Cc1cccc(NC(=O)c2ccc(-n3cc(CCCCC(=O)O)nn3)cc2)c1 ; Cc1ncsc1-c1ccc([C@H](C)NC(=O)[C@@H]2C[C@@H](O)CN2C(=O)[C@H](C)NC(=O)CCCCc2cn(-c3ccc(C(=O)Nc4cccc(C=C(C#N)C(=O)Nc5ccccc5C(=O)O)c4)cc3)nn2)cc1]

In a 10 mL round bottom flask, S16 (50 mg, 0.079 mmol) was dissolved in CH₃CN (3 ml), and then HATU (36 mg, 0.094 mmol), DIPEA (44 μL, 0.24 mmol), and HCl salt of E3 ligase ligand 1A (38 mg, 0.079 mmol) were added at room temperature. The reaction mixture was stirred for 45 min (monitored by LC-MS), the solvent was removed under the reduced pressure and purified by a silica gel ISCO combi flash column (CH₂Cl₂/MeOH) to provide tBu-XL5-VHL-2. The product was subjected to 1 mL CH₂Cl₂/TFA (1:1) and stirred for 1 hour at room temperature. The solvent and TFA were removed under the reduced pressure to provide the XL5-VHL-2 crude material. The crude material was purified by a preparatory HPLC with a XBridge BEH C18 OBD Prep Column, 130 Å, 5 μm, 30 mm×150 mm reverse-phase column as the stationary phase. Water (buffered with 0.05% trifluoroacetic acid) and MeCN were used as the mobile phase and HPLC conditions: UV collection 254 nm, flow rate 30 mL/min, 20% MeCN as linear gradient for 5 min and 20%→70% MeCN for 5 to 25 min. The HPLC fractions were combined and lyophilized to yield XL5-VHL-2 (26 mg, 33%, E/Z=3.5:1). ¹H NMR (400 MHz, DMSO-d₆): δ 12.24 (s, 1H), 10.67 (s, 1H), 8.98 (s, 1H), 8.71 (s, 1H), 8.64 (dd, J=8.5, 1.1 Hz, 1H), 8.51 (t, J=1.9 Hz, 1H), 8.41 (s, 1H), 8.37 (d, J=7.8 Hz, 1H), 8.20 (d, J=8.8 Hz, 2H), 8.14-8.02 (m, 3H), 8.00-7.95 (m, 1H), 7.87-7.78 (m, 2H), 7.70 (ddd, J=8.7, 7.3, 1.7 Hz, 1H), 7.62 (t, J=8.0 Hz, 1H), 7.43 (d, J=8.4 Hz, 2H), 7.37 (d, J=8.3 Hz, 2H), 7.27 (td, J=7.6, 1.2 Hz, 1H), 4.91 (t, J=7.2 Hz, 1H), 4.52 (d, J=9.3 Hz, 1H), 4.42 (t, J=8.0 Hz, 1H), 4.34-4.22 (m, 1H), 3.63-3.60 (m, 2H), 2.74 (t, J=7.2 Hz, 2H), 2.45 (s, 3H), 2.38-2.14 (m, 2H), 2.05-1.94 (m, 1H), 1.79 (ddd, J=12.7, 8.5, 4.5 Hz, 1H), 1.74-1.50 (m, 4H), 1.36 (d, J=6.9 Hz, 3H), 0.93 (s, 9H); ¹³C NMR (101 MHz, DMSO-d₆): δ 172.40, 171.08, 170.24, 170.08, 165.17, 159.47, 153.27, 151.99, 151.93, 148.84, 148.18, 145.12, 140.64, 140.26, 139.44, 134.84, 134.79, 134.37, 132.61, 131.73, 131.66, 131.59, 130.13, 130.06, 129.28, 126.84, 124.33, 122.51, 120.88, 120.69, 119.77, 117.50, 116.00, 106.65, 69.23, 59.01, 56.90, 48.21, 48.09, 38.18, 35.64, 35.06, 28.82, 26.92, 25.45, 25.20, 22.89, 16.39; HRMS (m/z): [M+H]⁺ calcd. for C₅₄H₅₇N₁₀O₈S, 1005.4082; found, 1005.4093.

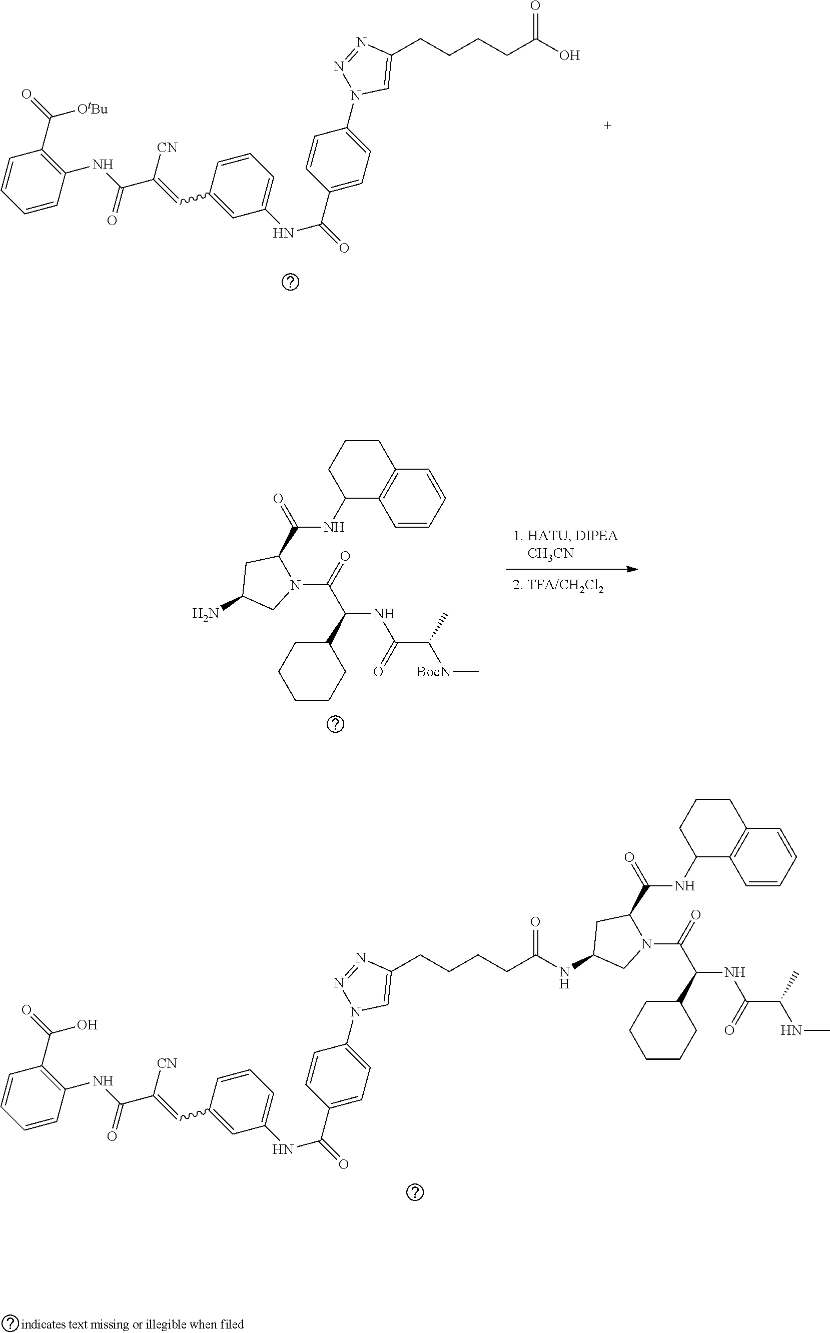 [SMILES: CC(C)(C)OC(=O)c1ccccc1NC(=O)C(C#N)=Cc1cccc(NC(=O)c2ccc(-n3cc(CCCCC(=O)O)nn3)cc2)c1 ; C[C@@H](C(=O)N[C@H](C(=O)N1C[C@@H](N)C[C@H]1C(=O)NC1CCCc2ccccc21)C1CCCCC1)N(C)C(=O)OC(C)(C)C ; CN[C@@H](C)C(=O)N[C@H](C(=O)N1C[C@@H](NC(=O)CCCCc2cn(-c3ccc(C(=O)Nc4cccc(C=C(C#N)C(=O)Nc5ccccc5C(=O)O)c4)cc3)nn2)C[C@H]1C(=O)NC1CCCc2ccccc21)C1CCCCC1]

XL5-IAP was synthesized using the same reaction sequence as XL5-VHL-2. Starting materials S16 (50 mg, 0.079 mmol), HATU (35 mg, 0.094 mmol), DIPEA (44 μL, 0.24 mmol), and HCl salt of S17 (50 mg, 0.079 mmol) were used. The crude material was purified by a preparatory HPLC with a XBridge BEH C18 OBD Prep Column, 130 Å, 5 μm, 30 mm×150 mm reversed-phase column as the stationary phase. Water (buffered with 0.05% trifluoroacetic acid) and MeCN were used as the mobile phase and HPLC conditions: UV collection 254 nm, flow rate 30 mL/min, 40% MeCN as linear gradient for 5 min and 40%→55% MeCN for 5 to 20 min. The HPLC fractions of major isomer were combined and lyophilized to yield XL5-IAP (22 mg, 28%). ¹H NMR (400 MHz, DMSO-d₆): δ 13.09 (s, 1H), 10.69 (s, 1H), 8.80-8.67 (m, 2H), 8.61 (d, J=8.3 Hz, 1H), 8.48 (d, J=2.0 Hz, 1H), 8.41 (d, J=8.7 Hz, 1H), 8.36 (s, 1H), 8.24-8.17 (m, 2H), 8.12-8.03 (m, 2H), 7.98 (dd, J=7.9, 2.0 Hz, 1H), 7.81 (d, J=7.9 Hz, 1H), 7.59 (t, J=7.9 Hz, 2H), 7.28 (d, J=7.5 Hz, 1H), 7.21 (t, J=7.6 Hz, 1H), 7.17-7.03 (m, 3H), 4.91 (t, J=6.6 Hz, 1H), 4.48-4.15 (m, 3H), 4.11 (t, J=8.6 Hz, 1H), 3.86 (t, J=6.8 Hz, 1H), 3.33 (t, J=8.8 Hz, 1H), 2.85-2.58 (m, 4H), 2.38 (dt, J=12.6, 7.6 Hz, 1H), 2.14 (t, J=7.0 Hz, 2H), 1.97-1.40 (m, 16H), 1.43-0.72 (m, 9H); ¹³C NMR (101 MHz, DMSO-d₆): δ 172.37, 171.23, 170.44, 169.75, 169.11, 165.11, 159.43, 152.69, 148.73, 140.66, 140.26, 139.42, 137.73, 137.43, 134.32, 133.80, 132.64, 131.72, 130.07, 129.01, 128.83, 127.12, 126.09, 125.28, 123.97, 122.57, 120.67, 120.46, 119.68, 116.04, 107.13, 58.89, 56.22, 55.94, 52.85, 48.16, 47.22, 35.61, 34.83, 31.26, 31.17, 30.24, 29.19, 28.92, 28.75, 28.59, 26.18, 25.96, 25.23, 25.09, 20.63, 16.20; HRMS (m/z): [M+H]⁺ calcd. for C₅₆H₅₆N₁₁O₈, 1044.5096; found, 1044.5090.

### Example 10: Applicability of Disclosed Compounds to Additional Cancer Types

The presence of hRpn13-Pru has been detected in cell lines from different cancer types, including multiple myeloma (RPMI 8226, MM.1S, LP1, KMS28BM), prostate (LNCaP) and pancreatic adenocarcinoma (ASPC-1), making them well-suited for the application of hRpn13-Pru PROTACs as an anti-cancer therapeutic strategy.

TABLE 2
Presence of hRpn13-Pru and Sensitivity
to XL5-VHL-2 of Various Cell Lines

| | Relative abundance of | XL5-VHL-2 Sensitivity (IC₅₀, |
|---|---|---|
| Cell Line | hRpn13-Pru (%)^{a} | μM) Cell viability assay |
| RPMI 8226 | 100 | 4.2 ± 1.0 |
| MM.1S | 64.00 | 5.9 ± 0.7 |
| NCl-H929 | 42.91 | Not tested |
| HCT116 | 8.33 | 23.6 ± 4.7 |
| SK-OV-3 | 3.51 | Not tested |
| Hs27 | Not detected | Not tested |
| LP1 | 53.97 | Not tested |
| LP1-R | 12.31 | Not tested |
| KMS28BM | 77.17 | Not tested |
| KMS28BM-R | 36.68 | Not tested |
| LNCaP | 101.13 | Not tested |
| DU-145 | 1.52 | Not tested |
| PC-3 | 1.89 | Not tested |
| MDA PCa2b | 54.22 | Not tested |
| VCaP | 13.92 | Not tested |
| 22RV1 | Not detected | Not tested |
| PSN-1 | 5.40 | Not tested |
| Panc-1 | 19.40 | Not tested |
| BxPC3 | 6.33 | Not tested |
| ASPC-1 | 121.89 | Not tested |
| Capan-1 | 35.86 | Not tested |
| MiaPaca | 1.05 | Not tested |

^{a}Relative abundance of hRpn13-Pru (%) is calculated as the ratio of intensities for hRpn13-Pru normalized to hRpn13 full length (I_{hRpn13-Pru}/I_{hRpn13})ₛₐₘₚₗₑ divided by that of RPMI 8226 cells and multiplied by 100.

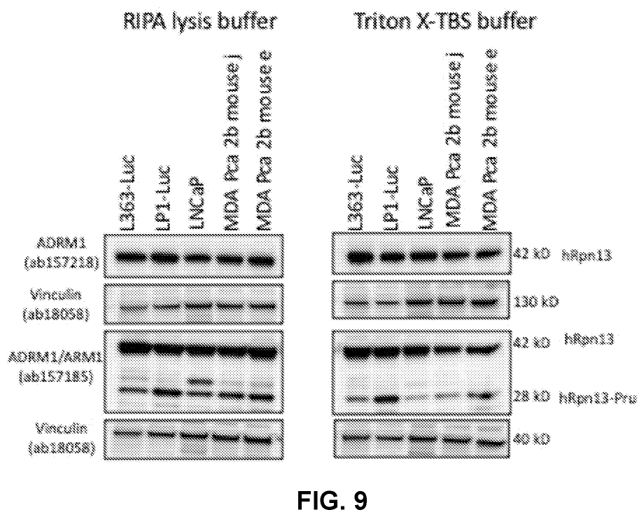

FIG. 9 shows that hRpn13-Pru is present in mouse xenograft models of prostate and pancreatic adenocarcinoma.

The hRpn13-Pru PROTAC built with XL5 can induce apoptosis with different linkers to connect XL5 to the E3 warhead. One linker contains a triazole group (XL5-VHL-2) whereas the others are linear (XL5-VHL-3, XL5-VHL-4, XL5-VHL-5). In all cases, the E3 warhead is VHL, and is best for RPMI 8226. For other cancer types, the E3 warhead can be replaced.

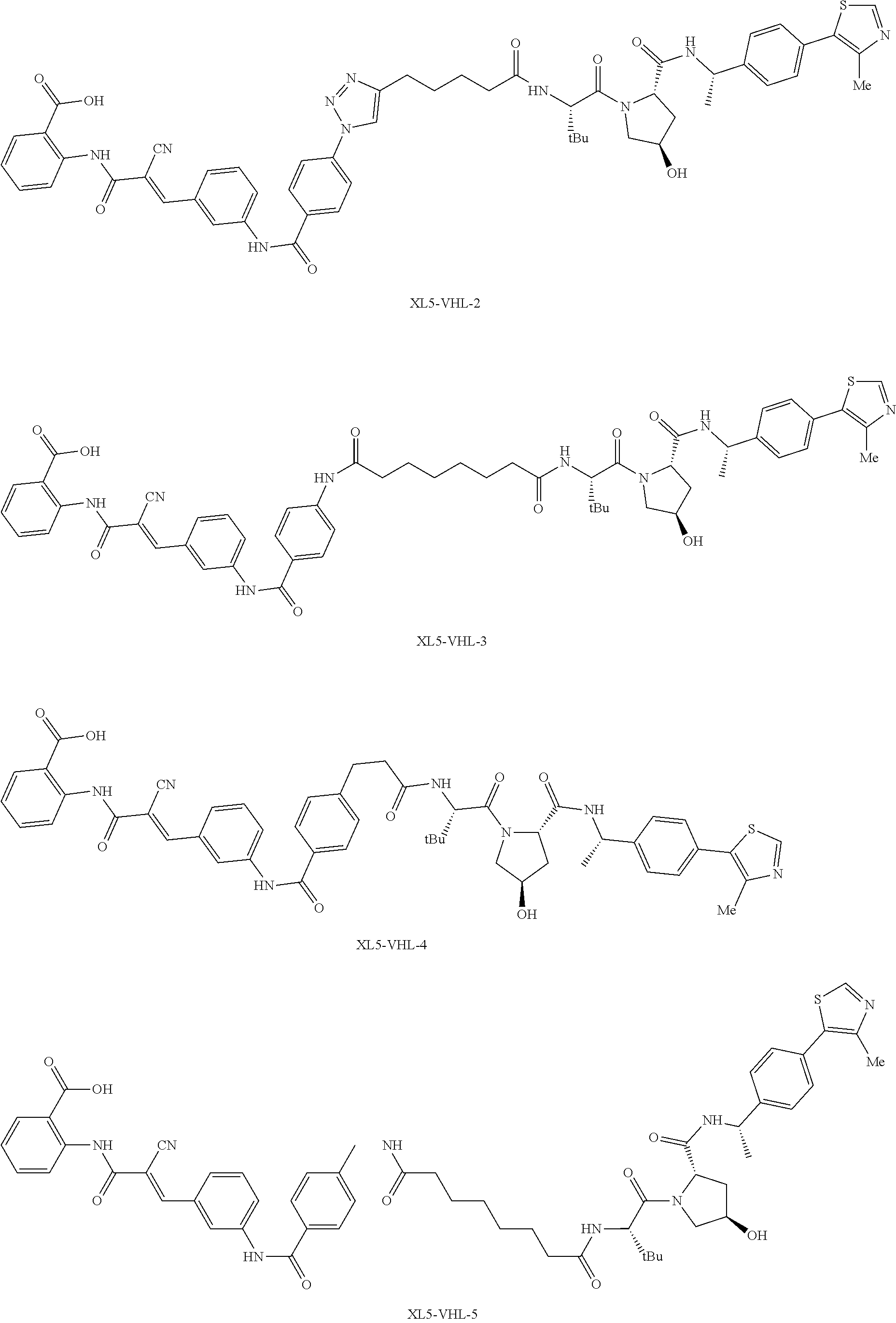

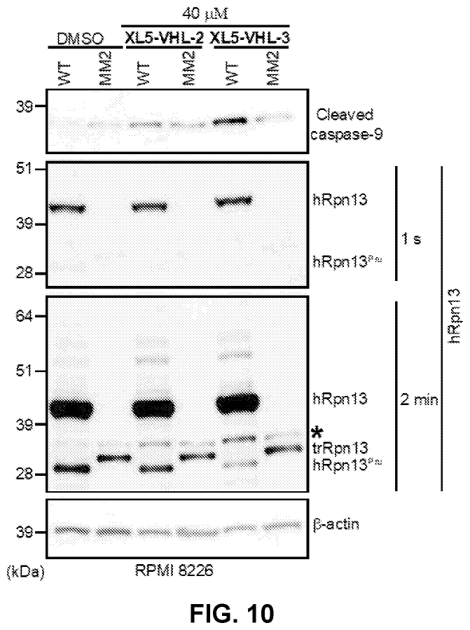

Each of these compounds induce apoptosis (FIG. 10, top panel, cleaved caspase-9) and loss of hRpn13-Pru in RPMI 8226 wild-type (WT) cells (FIG. 10, third panel) with apoptosis induction compromised, when cells are gene-edited to lose the hRpn13 Pru domain (trRpn13-MM2).

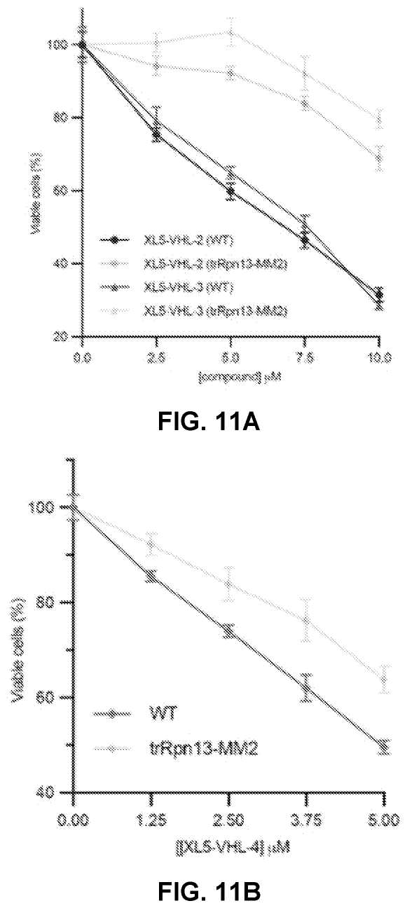

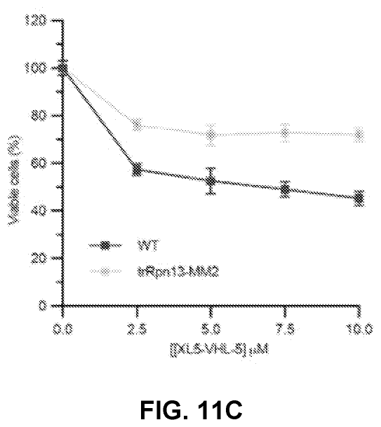

Cell viability of RPMI 8226 is also lost with treatment of either XL5-VHL-2, XL5-VHL-3, XL5-VHL-4 or XL5-VHL-5 and the effect hRpn13 dependent, particularly for XL5-VHL-2 and XL5-VHL-3, based on loss of activity in trRpn13-MM2 cells (FIGS. 11A-11C).

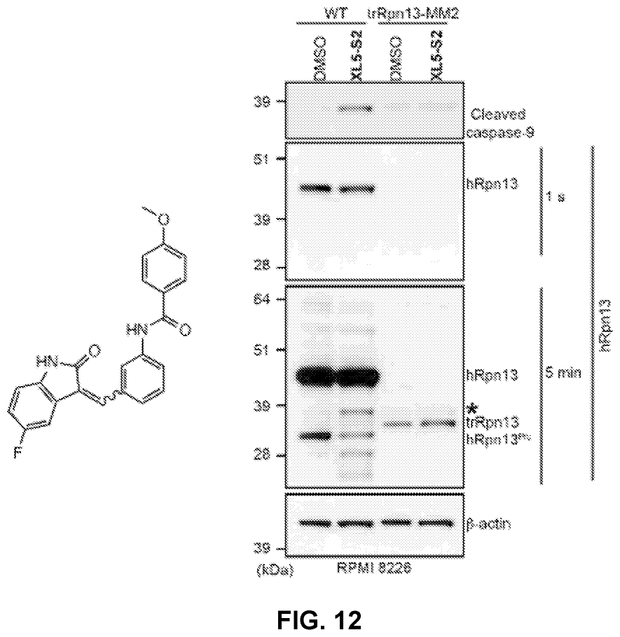

By using the solved hRpn13-Pru:XL5 structure, a virtual screen was performed that led to the discovery of a variant of XL5 (named XL5-S2, FIG. 12, left panel) that induces apoptosis in RPMI 8226 cells in an hRpn13-dependent manner (FIG. 12, right panel).

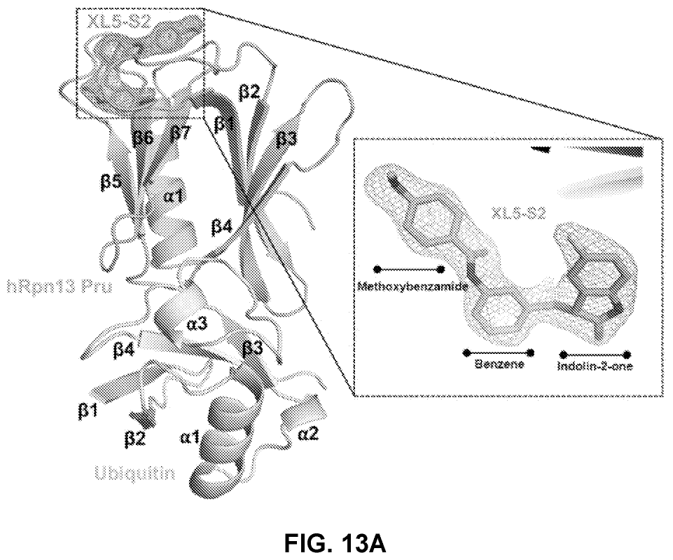

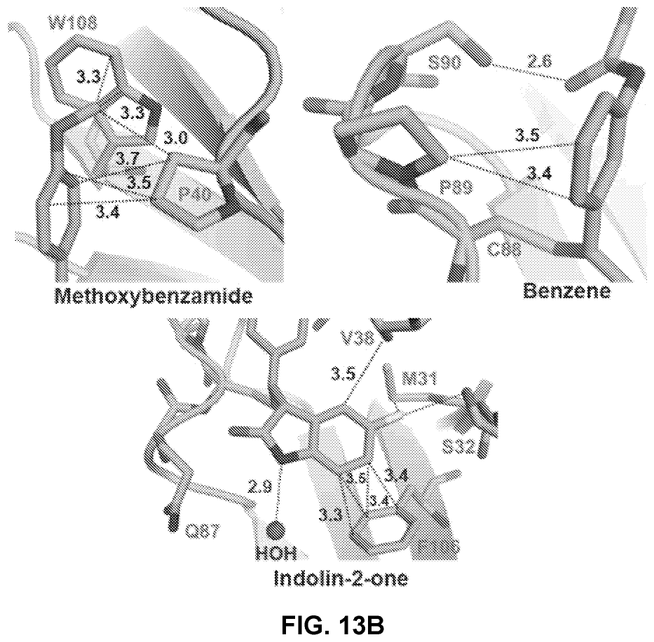

The structure of hRpn13-Pru with XL5-S2 was solved, identifying key interactions as well as positions that can be modified for optimized binding (see FIGS. 13A-13B).

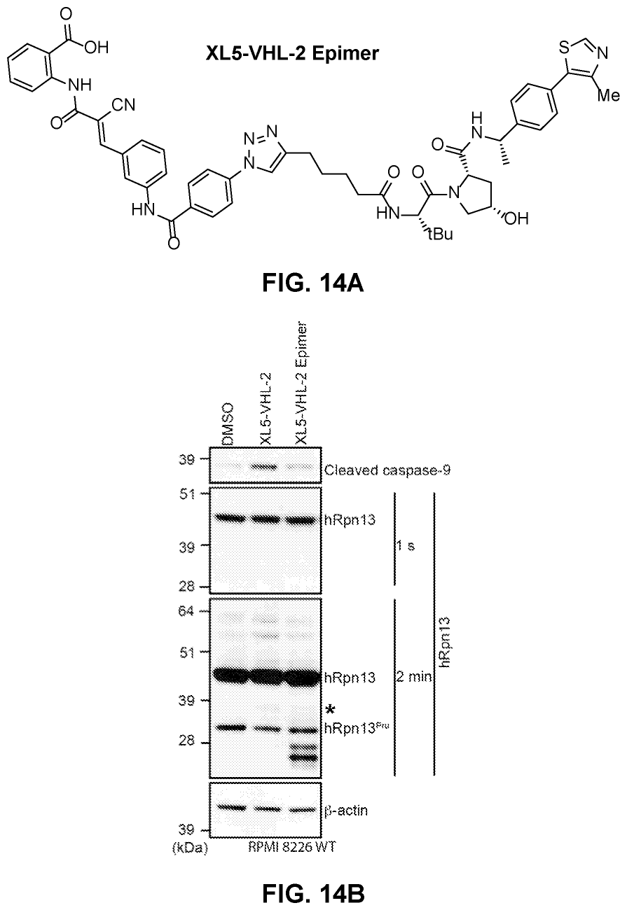

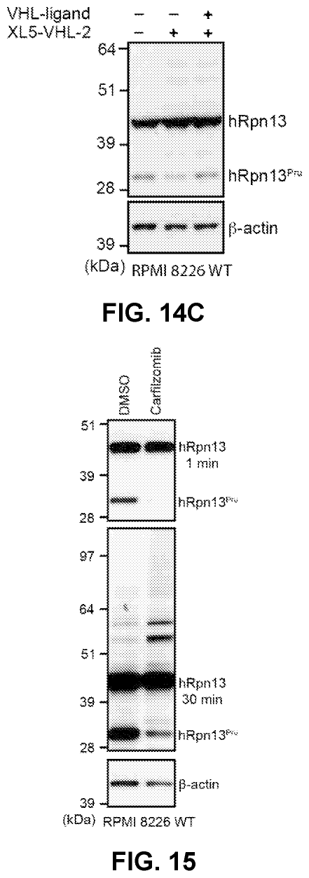

By performing a competition experiment with VHL ligand (FIG. 14C) and by using an epimer with a VHL-inactive degrader (FIG. 14B), it was demonstrated that degradation of hRpn13-Pru by XL5-VHL-2 is through VHL.

By treating cells with a proteasome inhibitor (carfilzomib) and observing reduced levels of hRpn13-Pru, it was shown that hRpn13-Pru is generated by the proteasome cleaving the full-length protein (FIG. 15).

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Anchoori, R. K. et al. A bis-benzylidine piperidone targeting proteasome ubiquitin receptor RPN13/ADRM1 as a therapy for cancer. Cancer cell 24, 791-805, doi:10.1016/j.ccr.2013.11.001 (2013).
2. Anchoori, R. K. et al. Covalent Rpn13-Binding Inhibitors for the Treatment of Ovarian Cancer. ACS Omega 3, 11917-11929, doi:10.1021/acsomega.8b01479 (2018).
3. Anchoori, R. K. et al. Structure-function analyses of candidate small molecule RPN13 inhibitors with antitumor properties. PloS one 15, e0227727, doi:10.1371/journal.pone.0227727 (2020).
4. Bartels, C., et al. The program XEASY for computer-supported NMR spectral analysis of biological macromolecules. Journal of biomolecular NMR 6, 1-10, doi:10.1007/BF00417486 (1995).
5. Biancospino, M. et al. Clathrin light chain A drives selective myosin VI recruitment to clathrin-coated pits under membrane tension. Nat Commun 10, 4974, doi:10.1038/s41467-019-12855-6 (2019).
6. Bradshaw, J. M. et al. Prolonged and tunable residence time using reversible covalent kinase inhibitors. Nat Chem Biol 11, 525-531, doi:10.1038/nchembio.1817 (2015).
7. Buel, G. R. et al. Structure of E3 ligase E6AP with a proteasome-binding site provided by substrate receptor hRpn10. Nat Commun 11, 1291, doi:10.1038/s41467-020-15073-7 (2020).
8. Chen, X. et al. Cryo-EM Reveals Unanchored M1-Ubiquitin Chain Binding at hRpn11 of the 26S Proteasome. Structure, doi:10.1016/j.str.2020.07.011 (2020).
9. Chen, X., et al. Identifying and studying ubiquitin receptors by NMR. Methods Mol Biol 832, 279-303, doi:10.1007/978-1-61779-474-2_20 (2012).
10. Chen, X. et al. Structures of Rpn1 T1:Rad23 and hRpn13:hPLIC2 Reveal Distinct Binding Mechanisms between Substrate Receptors and Shuttle Factors of the Proteasome. Structure 24, 1257-1270, doi:10.1016/j.str.2016.05.018 (2016).
11. Chen, X., et al. Proteasome interaction with ubiquitinated substrates: from mechanisms to therapies. FEBS J, 407 doi:10.1111/febs.15638 (2020).
12. Chen, X., et al. Structure of proteasome ubiquitin receptor hRpn13 and its activation by the scaffolding protein hRpn2. Molecular cell 38, 404-415, doi:10.1016/j.molcel.2010.04.019 (2010).
13. Cromm, P. M. et al. The Proteasome in Modern Drug Discovery: Second Life of a Highly Valuable Drug Target. ACS Cent Sci 3, 830-838, 410 doi:10.1021/acscentsci.7b00252 (2017).
14. de la Pena, A. H., et al. Substrate-engaged 26S proteasome structures reveal mechanisms for ATP-hydrolysis-driven translocation. Science 362, doi:10.1126/science.aav0725 (2018).
15. Delaglio, F. et al. NMRPipe: a multidimensional spectral processing system based on UNIX pipes. Journal of biomolecular NMR 6, 277-293 (1995).
16. Deol, K. K. et al. Proteasome-Bound UCH37/UCHL5 Debranches Ubiquitin Chains to Promote Degradation. Mol Cell, doi:10.1016/j.molcel.2020.10.017 (2020).
17. Dickson, P. et al. Physical and Functional Analysis of the Putative Rpn13 Inhibitor RA190. Cell Chem Biol, doi:10.1016/j.chembiol.2020.08.007 (2020).
18. Dickson, P., et al. Mechanistic Studies of the Multiple Myeloma and Melanoma Cell-Selective Toxicity of the Rpn13-Binding Peptoid 480 KDT-11. Cell Chem Biol, doi:10.1016/j.chembiol.2020.08.008 (2020).
19. Dong, Y. et al. Cryo-EM structures and dynamics of substrate-engaged human 26S proteasome. Nature 565, 49-55, doi:10.1038/s41586-018-0736-4 (2019).
20. Gabizon, R. et al. Efficient Targeted Degradation via Reversible and Irreversible Covalent PROTACs. J Am Chem Soc 142, 11734-11742, doi:10.1021/jacs.9b13907 (2020).
21. Guo, W. H. et al. Enhancing intracellular accumulation and target engagement of PROTACs with reversible covalent chemistry. Nat Commun 11, 4268, doi:10.1038/s41467-020-17997-6 (2020).
22. Hallenbeck, K. K., et al. Targeting Non-Catalytic Cysteine Residues Through Structure-Guided Drug Discovery. Curr Top Med Chem 17, 4-15 (2017).
23. Hamazaki, J. et al. A novel proteasome interacting protein recruits the deubiquitinating enzyme UCH37 to 26S proteasomes. The EMBO journal 25, 4524-4536, doi:10.1038/sj.emboj.7601338 (2006).
24. He, F. et al. Myosin VI Contains a Compact Structural Motif that Binds to Ubiquitin Chains. Cell Rep 14, 2683-2694, doi:10.1016/j.celrep.2016.01.079 (2016).
25. Hiyama, H. et al. Interaction of hHR23 with S5a. The ubiquitin-like domain of hHR23 mediates interaction with S5a subunit of 26 S proteasome. J Biol Chem 274, 28019-28025 (1999).
26. Hu, J. et al. Discovery of ERD-308 as a Highly Potent Proteolysis Targeting Chimera (PROTAC) Degrader of Estrogen Receptor (ER). J Med Chem 62, 1420-1442, doi:10.1021/acs.jmedchem.8b01572 (2019).
27. Husnjak, K. et al. Proteasome subunit Rpn13 is a novel ubiquitin receptor. Nature 453, 421 481-488, doi:10.1038/nature06926 (2008).
28. Koradi, R., et al. MOLMOL: a program for display and analysis of macromolecular structures. Journal of molecular graphics 14, 51-55, 29-32 (1996).
29. Kumar, C. D. et al. Investigation of anion-pi interactions involving thiophene walls incorporated calix[4]pyrroles. J Org Chem 80, 1746-1753, doi:10.1021/jo502673c 506 (2015).
30. Laskowski, R. A., et al. AQUA and PROCHECK-NMR: programs for checking the quality of protein structures solved by NMR. Journal of biomolecular NMR 8, 477-486 (1996).
31. Lauinger, L. et al. Thiolutin is a zinc chelator that inhibits the Rpn11 and other JAMM metalloproteases. Nat Chem Biol 13, 709-714, doi:10.1038/nchembio.2370 (2017).
32. Laurent, A., et al. Functionalized pyrrolidines and use thereof as IAP inhibitors. PCT Int. Appl. (2009).
33. Leggett, D. S. et al. Multiple associated proteins regulate proteasome structure and function. Molecular cell 10, 495-507 (2002).
34. Lu, X. et al. Structure-guided bifunctional molecules hit a DEUBAD-lacking hRpn13 species upregulated in multiple myeloma. Nat Commun, 12, 7318 (2021).
35. Lu, X. et al. Structure of the Rpn13-Rpn2 complex provides insights for Rpn13 and Uch37 as anticancer targets. Nat Commun 8, 15540, doi:10.1038/ncomms15540 (2017).
36. Lu, X., et al. A High Affinity hRpn2-Derived Peptide That Displaces Human Rpn13 from Proteasome in 293T Cells. PloS one 10, e0140518, doi:10.1371/journal.pone.0140518 (2015).
37. Lu, X., et al. An Extended Conformation for K48 Ubiquitin Chains Revealed by the hRpn2:Rpn13:K48-Diubiquitin Structure. Structure 28, 495-506 e493, doi:10.1016/j.str.2020.02.007 (2020).
38. Neves, M. A., et al. Docking and scoring with ICM: the benchmarking results and strategies for improvement. J Comput Aided Mol Des 26, 675-686, doi:10.1007/s10822-012-9547-0 (2012).
39. Osei-Amponsa, V. et al. Impact of losing hRpn13 Pru or UCHL5 on proteasome clearance of ubiquitinated proteins and RA190 cytotoxicity. Mol Cell Biol, doi:10.1128/MCB.00122-20 (2020).
40. Perez, C. et al. Discovery of an Inhibitor of the Proteasome Subunit Rpn11. J Med Chem 60, 1343-1361, doi:10.1021/acs.jmedchem.6b01379 (2017).
41. Pettersson, M. et al. PROteolysis TArgeting Chimeras (PROTACs)—Past, present and future. Drug Discov Today Technol 31, 15-27, 415 doi:10.1016/j.ddtec.2019.01.002 (2019).
42. Qiu, X. B. et al. hRpn13/ADRM1/GP110 is a novel proteasome subunit that binds the deubiquitinating enzyme, UCH37. The EMBO journal 25, 5742-5753, doi:10.1038/sj.emboj.7601450 (2006).
43. Randles, L. et al. Ubiquitin and its binding domains. Front Biosci (Landmark Ed) 17, 2140-2157 (2012).
44. Roelofs, J. The Extent of Extended-Ubiquitin Binding to the Proteasome. Structure 28, 489-491, doi:10.1016/j.str.2020.04.013 (2020).
45. Schreiner, P. et al. Ubiquitin docking at the proteasome through a novel pleckstrin-homology domain interaction. Nature 453, 548-552, doi:10.1038/nature06924 (2008).
46. Schuttelkopf, A. W. et al. PRODRG: a tool for high-throughput crystallography of protein-ligand complexes. Acta Crystallogr D Biol Crystallogr 60, 1355-1363, doi:10.1107/S0907444904011679 (2004).
47. Schwieters, C. D., et al. The Xplor-NIH NMR molecular structure determination package. J Magn Reson 160, 65-73, doi:10.1016/s1090-7807(02)00014-9 (2003).
48. Serafimova, I. M. et al. Reversible targeting of noncatalytic cysteines with chemically tuned electrophiles. Nature chemical biology 8, 471-476, doi:10.1038/nchembio.925 (2012).
49. Shi, Y. et al. Rpn1 provides adjacent receptor sites for substrate binding and deubiquitination by the proteasome. Science 351, doi:10.1126/science.aad9421 (2016).
50. Song, Y. et al. Targeting proteasome ubiquitin receptor Rpn13 in multiple myeloma.

Leukemia 30, 1877-1886, doi:10.1038/leu.2016.97 (2016).
51. Trader, D. J., et al. A Reversible and Highly Selective Inhibitor of the Proteasomal Ubiquitin Receptor Rpn13 Is Toxic To Multiple Myeloma Cells. J. Am. Chem. Soc., doi:10.1021/jacs.5b02069 (2015).
52. VanderLinden, R. T., et al. Structure and energetics of pairwise interactions between proteasome subunits RPN2, RPN13, and ubiquitin clarify a substrate recruitment mechanism. J Biol Chem 292, 9493-9504, doi:10.1074/jbc.M117.785287 (2017).
53. Varfolomeev, E. et al. IAP antagonists induce autoubiquitination of c-IAPs, NF-kappaB activation, and TNFalpha-dependent apoptosis. Cell 131, 669-681, doi:10.1016/j.cell.2007.10.030 (2007).
54. Verma, R. et al. Proteasomal proteomics: identification of nucleotide-sensitive proteasome-interacting proteins by mass spectrometric analysis of affinity-purified proteasomes.

Mol Biol Cell 11, 3425-3439, doi:10.1091/mbc.11.10.3425 (2000).
55. Walters, K. J., et al. Structural studies of the interaction between ubiquitin family proteins and proteasome subunit S5a. Biochemistry 41, 1767-1777 (2002).
56. Wang, Q., et al Structure of S5a bound to monoubiquitin provides a model for polyubiquitin recognition. J Mol Biol 348, 727-739, doi:10.1016/j.jmb.2005.03.007 (2005).
57. Wollscheid, H. P. et al. Diverse functions of myosin VI elucidated by an isoform-specific α-helix domain. Nat Struct Mol Biol 23, 300-308, doi:10.1038/nsmb.3187 (2016).
58. Yao, T. et al. Proteasome recruitment and activation of the Uch37 deubiquitinating enzyme by Adrm1. Nature cell biology 8, 994-1002, doi:10.1038/ncb1460 (2006).
59. You, K. T. et al. Selective translational repression of truncated proteins from frameshift mutation-derived mRNAs in tumors. PLoS Biol 5, e109, doi:10.1371/journal.pbio.0050109 (2007).
60. Young, P., et al. Characterization of two polyubiquitin binding sites in the 26 S protease subunit 5a. J Biol Chem 273, 424 5461-5467 (1998).
61. Zhang, N. et al. Structure of the s5a:k48-linked diubiquitin complex and its interactions with rpn13. Mol Cell 35, 280-290, doi:10.1016/j.molcel.2009.06.010 (2009).

1. A compound comprising a structure of Formula I: 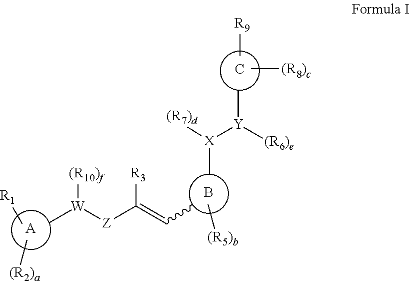
wherein A, B, and C independently comprise an aryl or heteroaryl ring having 5-10 members;
wherein X and Y independently comprise carbon, oxygen, nitrogen, sulfur, a carbonyl group, or a sulfonyl group;
wherein each instance of R₆ and R₇ is absent or independently comprises hydrogen, halogen, hydroxyl, trifluoromethyl, C₁-C₆ alkylamino, C₁-C₆ alkoxy, substituted or unsubstituted C₁-C₆ alkyl, a substituted or unsubstituted phenyl group, or Formula II; 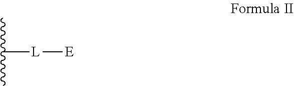
wherein L comprises a linker moiety;
wherein E comprises an E3 ubiquitin ligase targeting moiety, a bridging molecule to a ubiquitin E3 ligase complex, an E2 ubiquitin conjugating enzyme targeting molecule, an autophagy-targeting chimera, or a proteasome subunit targeting molecule;
wherein when at least one R₇ is present, d is 1 or 2; and
wherein when at least one R₆ is present, e is 1 or 2;
wherein R₁ comprises —SO₂NH₂, a carboxylic acid group, fluorine, a trifluoromethyl group, or a tetrazole;
wherein each instance of R₂ independently comprises hydrogen, halogen, hydroxyl, trifluoromethyl, C₁-C₆ alkylamino, C₁-C₆ alkoxy, or substituted or unsubstituted C₁-C₆ alkyl, and a is from 1 to 4;
wherein R₃ comprises a cyano group, —S(═O)₂—R₄, —C(═O)—R₄, —C(═O)—OR₄, —C(═O)—N—R₄,R₄′, or —S(═O)₂—NH₂;
wherein R₄ and R₄′ comprise hydrogen, halogen, hydroxyl, trifluoromethyl, C₁-C₆ alkylamino, C₁-C₆ alkoxy, substituted or unsubstituted C₁-C₆ alkyl, or a substituted or unsubstituted phenyl group;
wherein each instance of R₆ independently comprises hydrogen, halogen, hydroxyl, trifluoromethyl, C₁-C₆ alkylamino, C₁-C₆ alkoxy, substituted or unsubstituted C₁-C₆ alkyl, a substituted or unsubstituted phenyl group, or Formula II, and wherein b is from 1 to 5;
wherein each instance of Rₐ independently comprises hydrogen, halogen, hydroxyl, trifluoromethyl, C₁-C₆ alkylamino, C₁-C₆ alkoxy, or substituted or unsubstituted C₁-C₆ alkyl; and wherein c is from 1 to 5; and
wherein R₉ comprises hydrogen, halogen, hydroxyl, trifluoromethyl, substituted or unsubstituted C₁-C₆ alkylamino, substituted or unsubstituted C₁-C₆ alkyl, an azide group, or Formula II;
wherein Z comprises a carbonyl group or a sulfonyl group;
wherein W comprises carbon, oxygen, nitrogen, or sulfur;
wherein each instance of R₁₀ is absent or independently comprises hydrogen, halogen, hydroxyl, trifluoromethyl, C₁-C₆ alkylamino, a substituted or unsubstituted phenyl group, or Formula II; and wherein f is from 0 to 2; and
wherein the compound is not XL5 and is not XL23.

2. The compound of claim 1, wherein the compound comprises Formula Ia, Formula Ib, or any combination thereof:

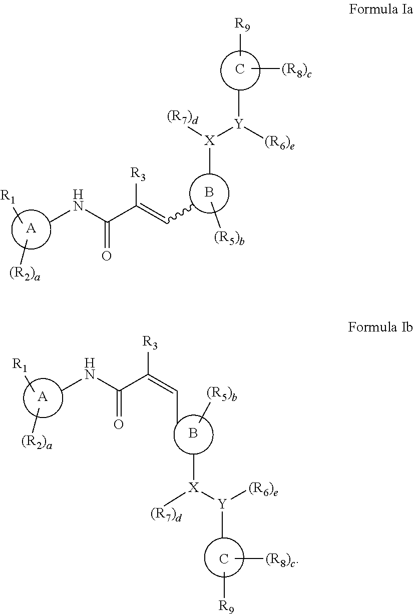

3. The compound of claim 1, wherein A and B are independently a substituted or unsubstituted phenyl group.

4. (canceled)

5. The compound of claim 1, wherein C is a substituted or unsubstituted phenyl or pyridyl group.

6. The compound of claim 1, wherein X is nitrogen.

7. The compound of claim 1, wherein R₇ is hydrogen and d is 1.

8. The compound of claim 1, wherein Y is a carbonyl group and R₆ is absent.

9. The compound of claim 1, wherein R₁ is —SO₂NH₂ or a carboxylic acid group.

10. The compound of claim 1, wherein each R₂ is independently hydrogen, trifluoromethyl, methylamino, or methoxy, and wherein a is 4.

11. The compound of claim 1, wherein R₃ is cyano.

12. The compound of claim 1, wherein each R₅ is independently hydrogen, trifluoromethyl, or methylamino and wherein b is 4.

13. The compound of claim 1, wherein each Ra is independently chloro, hydrogen, or hydroxyl, and wherein c is 4.

14. The compound of claim 1, wherein R₉ is hydrogen, methyl, methylamino, trifluoromethyl, —NHCH₂COOH, or Formula II.

15. The compound of claim 1, wherein at least one of R₅, R₆, R₇, or R₉ comprises Formula II and wherein L comprises: 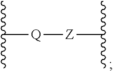
wherein Q comprises a triazole, an amide, a C₁-C₄ alkyl amide, a sulfonamide, or substituted or unsubstituted spirocyclic rings;
and wherein Z comprises an alkyl group, an alkylene group, a polyether group, or any combination thereof.

16. The compound of claim 15, wherein Z comprises: 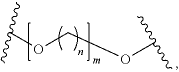
wherein n is 2 or 3 and wherein m is from 1 to 10; or 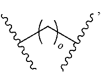
wherein o is from 0 to 10.

17. The compound of claim 1, wherein R₉ is Formula II and wherein L is: 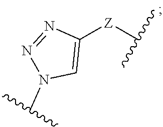
and wherein Z is an alkyl group, an alkylene group, a polyether group, or any combination thereof.

18. The compound of claim 1, wherein R₉ is Formula II and wherein L is: 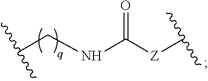
wherein q is 0 or 1;
and wherein Z is an alkyl group, an alkylene group, a polyether group, or any combination thereof.

19. The compound of claim 1, wherein R₉ is Formula II and wherein L is: 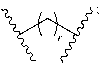
and wherein r is from 1 to 5.

20-27. (canceled)

28. The compound of claim 1 having a structure represented by a formula:

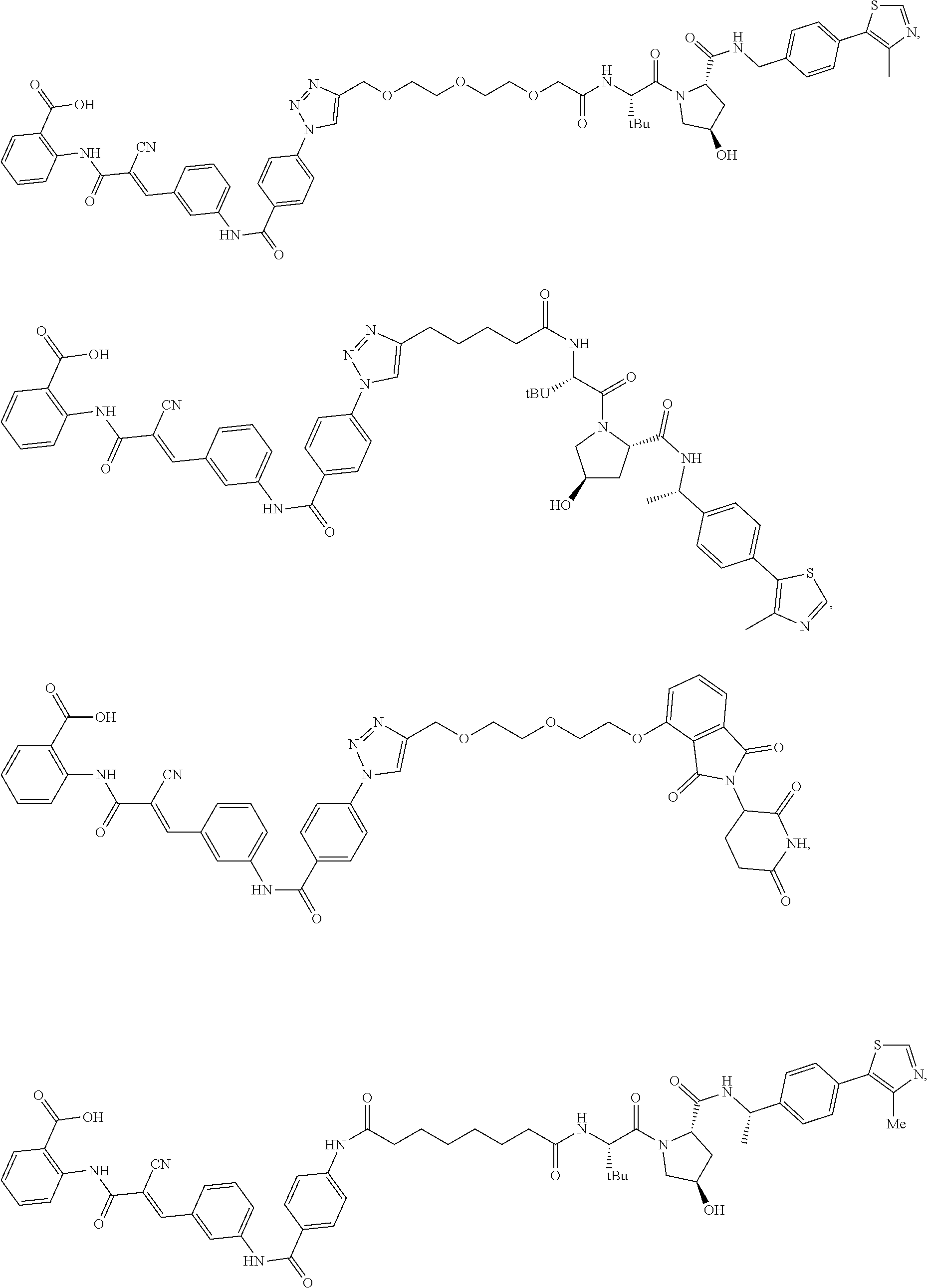

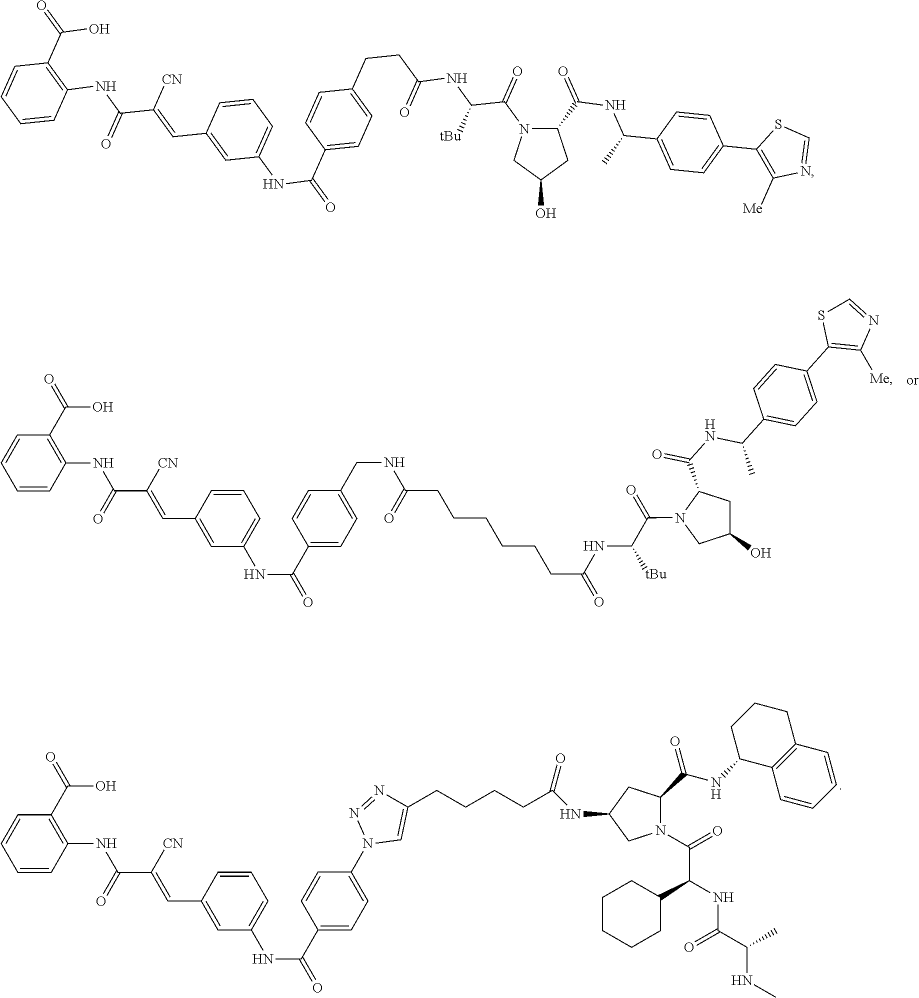 [SMILES: Cc1ncsc1-c1ccc([C@H](C)NC(=O)[C@@H]2C[C@@H](O)CN2C(=O)[C@@H](NC(=O)CCc2ccc(C(=O)Nc3cccc(/C=C(\C#N)C(=O)Nc4ccccc4C(=O)O)c3)cc2)C(C)(C)C)cc1 ; Cc1ncsc1-c1ccc([C@H](C)NC(=O)[C@@H]2C[C@@H](O)CN2C(=O)[C@@H](NC(=O)CCCCCCC(=O)NCc2ccc(C(=O)Nc3cccc(/C=C(\C#N)C(=O)Nc4ccccc4C(=O)O)c3)cc2)C(C)(C)C)cc1 ; CN[C@@H](C)C(=O)N[C@H](C(=O)N1C[C@@H](NC(=O)CCCCc2cn(-c3ccc(C(=O)Nc4cccc(/C=C(\C#N)C(=O)Nc5ccccc5C(=O)O)c4)cc3)nn2)C[C@H]1C(=O)N[C@@H]1CCCc2ccccc21)C1CCCCC1]

29. The compound of claim 1, having a structure represented by a formula:

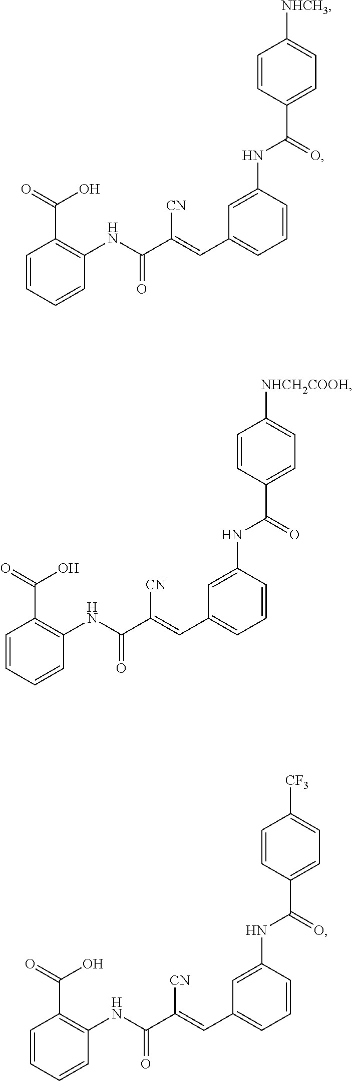

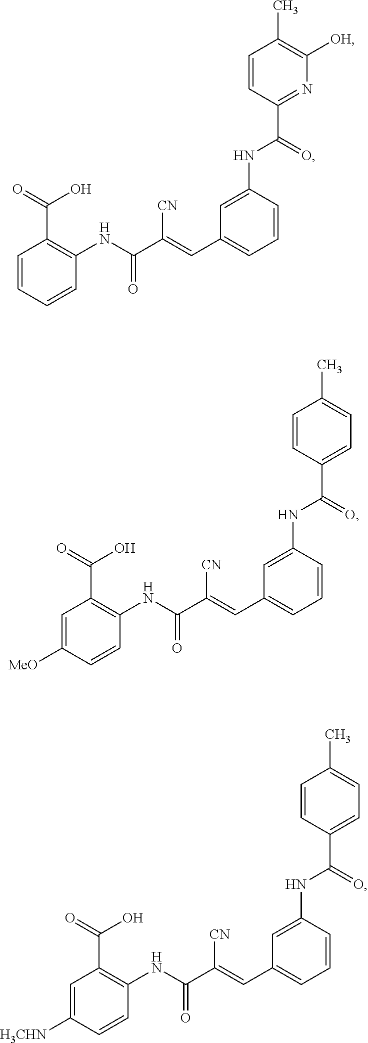

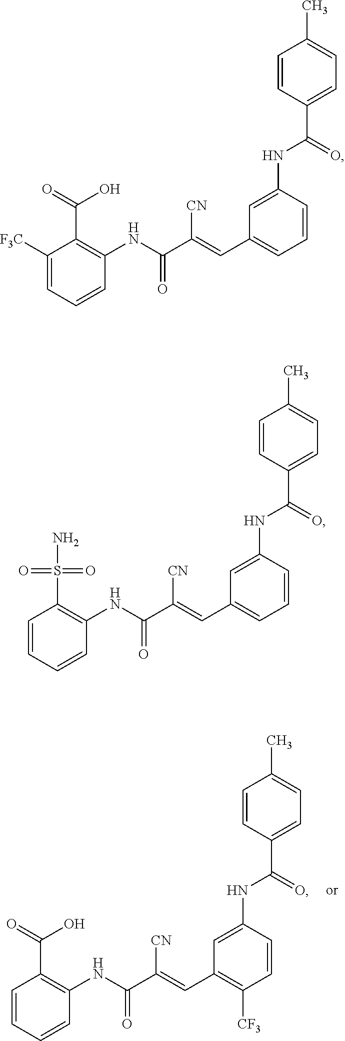 [SMILES: Cc1ccc(C(=O)Nc2cccc(/C=C(\C#N)C(=O)Nc3cccc(C(F)(F)F)c3C(=O)O)c2)cc1 ; Cc1ccc(C(=O)Nc2cccc(/C=C(\C#N)C(=O)Nc3ccccc3S(N)(=O)=O)c2)cc1 ; Cc1ccc(C(=O)Nc2ccc(C(F)(F)F)c(/C=C(\C#N)C(=O)Nc3ccccc3C(=O)O)c2)cc1]

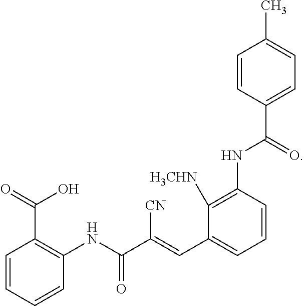

30-58. (canceled)
